(19) United States
(12) Patent Application Publication
(10) Pub. No.: US 2006/0111297 A1
(43) Pub. Date: May 25, 2006

(54) Blood factor domains

(76) Inventor: Bruce L. Roberts, Southboro, MA (US)

Correspondence Address: GENZYME CORPORATION;LEGAL DEPARTMENT, 15 PLEASANT ST CONNECTOR, FRAMINGHAM, MA, 01701-9322, US

(21) Appl. No.: 11/297,134

(22) Filed: Dec. 8, 2005

(30) Foreign Application Priority Data
Jun. 9, 2004 (WO) PCT/US04/18461

(51) Int. Cl. A61K 38/18; C07K 14/74; C07K 16/28; C07H 21/04; C12P 21/06

(52) U.S. Cl. 514/12; 435/325; 435/69.1; 435/320.1; 530/350; 536/23.5; 530/388.25

(57) ABSTRACT

The present invention provides methods and compositions for detecting, diagnosing, prognosing and monitoring the progress of diseases. Further provided are methods for screening to identify agonists and antagonists of antigens associated with these diseases.

### CROSS-REFERENCE TO RELATED APPLICATIONS

This application claims priority to PCT Application No. PCT/US2004/018461, filed 9 Jun. 2004, which claims priority to U.S. Provisional Application No. 60/477,291, filed Jun. 9, 2003, the contents of which hereby incorporated by reference into the present disclosure.

### TECHNICAL FIELD

This invention is in the field of immunology and immunotherapy. In particular, the present invention provides compositions that act as cytokines and/or chemokines which useful in modulating the immune response.

### BACKGROUND

The mammalian immune system comprises two types of antigen-specific cells: B cells and T cells. B cells synthesize both membrane-bound and secreted antibody. T cells can be characterized phenotypically by the manner in which they recognize antigen, by their cell surface markers, and by their secreted products. T cells express distinctive membrane molecules. Included among these are the T cell antigen receptor (TCR), which appears on the cell surface in association with CD3; and accessory molecules such as CD5, CD28 and CD45R. Subpopulations of T cells can be distinguished by the presence of additional membrane molecules. Thus, for example, T cells that express CD4 recognize antigen associated with Class II MHC molecules and generally function as helper cells whose roles include enhancement of antibody production by B cells, while T cells that express CD8 recognize antigen associated with Class I MHC molecules and generally function as cytotoxic cells.

Immune cells recognize discrete sites, known as epitopes or antigenic determinants, on the antigen. Epitopes are regions of an immunogen or antigen that bind to antigen-specific membrane-bound receptors on immune cells or to their soluble counterparts, such as antibodies. Both membrane-bound antibody on the surface of a B lymphocyte and secreted antibody recognize soluble antigen. Unlike B cells, which recognize soluble antigen, T cells recognize antigen only when the antigen is associated with self major histocompatibility complex (MHC) gene products on the surface of an antigen presenting cell. This antigen can be displayed together with MHC molecules on the surface of antigen-presenting cells or on virus-infected cells, cancer cells, and grafts.

Disease states can result from invasion by a pathogenic organisms, including bacterial, viral, and protozoan pathogens, and subsequent inefficient or ineffective immune response to the invader. Disease states can also result from the activation of self-reactive T lymphocytes, from the activation of T lymphocytes that provoke allergic reactions, or from the activation of autoreactive T lymphocytes following certain bacterial and parasitic infections, which can produce antigens that mimic human protein, rendering these protein “autoantigens.” These diseases include, but are not limited to, the autoimmune diseases, autoimmune disorders that occur as a secondary event to infection with certain bacteria or parasites, T cell mediated allergies, and certain skin diseases such as psoriasis and vasculitis. Furthermore, undesired rejection of a foreign antigen can result in graft rejection or even infertility, and such rejection may be due to activation of specific T lymphocyte populations.

Small, soluble proteins have also been shown to critical to the immune response. Although lymphocytes, macrophages, and granulocytes play a major role in the response, the soluble proteins, known as lymphokines, cytokines, or monokines, and secondly, chemokines, are now known to play a critical role.

Cytokines exhibit a wide variety of functions. A hallmark feature is their ability to elicit chemotactic migration of distinct cell types, including polymorphonuclear cells and macrophages. Many cytokines have pro-inflammatory activity and are involved in multiple steps during inflammatory reactions. They also have been implicated in a number of physiological and disease conditions, including lymphocyte trafficking, wound healing, hematopoietic regulation and immunological disorders such as allergy, asthma and arthritis.

Chemokines are a family of small cytokines thought to mediate the directional migration of specific target populations of leukocytes along concentration gradients through the endothelial cell layer to the site of lesion. Baggiolini et al. (1994) Adv. Immunol. 55: 97-179. This cascade of events, however can display a high degree of specificity in relation to the inflammatory stimulus, the stage of the inflammatory response and the tissue or organ involved. Butcher et al. (1991) Cell 67:1033-1036.

### DISCLOSURE OF THE INVENTION

This invention provides compositions and methods for modulating an immune response. In one aspect, the immune response is a disease or condition related to a population of CD4⁺ or CD8⁺ T cells. The compositions identified in Table 1, infra, are immune response modulators exhibit chemokine and cytokine biological activity and therefore are useful to up-regulate immune response and alternatively, to down-regulate the same. Also provided are therapeutic agents that enhance or diminish the native activity of the immune modulators.

This invention provides isolated proteins and/or polypeptides useful in the methods identified herein. Further provided are polynucleotides encoding the proteins, fragments thereof, or polypeptides, (also referred to herein as gene expression product), gene delivery vehicles comprising these polynucleotides and host cells comprising these polynucleotides. Polynucleotides of the invention are intended to include DNA, cDNA, RNA and genomic DNA. Expression systems, including gene delivery vehicles such as liposomes and vectors, and host cells containing the polynucleotides are further provided by this invention. The proteins, polypeptides or fragments thereof are also useful to generate antibodies that specifically recognize and bind to these molecules. The antibodies can be polyclonal or monoclonal. These antibodies can be used to isolate protein or polypeptides expressed from the genes identified in Table 1.

The invention also provides isolated host cells and recombinant host cells that contain a gene of Table 1 or its expression product and/or fragments thereof. The cells can be prokaryotic or eukaryotic and by way of example only, can be any one or more of bacterial, yeast, animal, mammalian, human, and particular subtypes thereof, e.g., stem cells, antigen presenting cells (APCs) such as dendritic cells (DCs) or T cells.

The present invention also provides proteins encoded by the polynucleotides.

Additionally, nucleic acid probes and primers that hybridize to invention polynucleotides are provided, as well as isolated nucleic acids comprising unique, expressed gene sequences.

The present invention further includes antisense oligonucleotides, antibodies, hybridoma cell lines and compositions containing same.

The present invention also provides methods of monitoring gene expression using invention polynucleotides. Also provided are compositions and methods to monitor expression of the polynucleotides and expression of the polynucleotides by detecting the expression products such as mRNA and/or polypeptides.

This invention further provides localized and systemic methods for: modulating the expression of the immunomodulatory polynucleotides and expression products, altering the activity of the proteins encoded by the polynucleotides, and treating symptoms of cancer, viral infection and auto-immune disorders.

This invention also provides a method for screening for candidate agents that modulate the expression of a polynucleotide or its complement and the expression products of the polynucleotide. The present invention also provides assays for the isolation of the ligand or ligands capable of modulating the activity of the invention polynucleotides and/or proteins.

This invention further provides assays for the identification, assessment and development of candidate agents capable of modulating the activity of the of the invention polynucleotides and/or proteins.

Further provided by this invention is a method for monitoring an immune response in a subject by assaying, at different times, the expression level of at least one gene identified in Table 1 and comparing the expression levels of the gene (transcript or expression product) to determine if expression has increased or decreased, thereby monitoring the disease or condition in the subject. A kit for use in a diagnostic method or drug screen is further provided herein. The kit comprises at least one agent (e.g., probe, primer or antibody) that detects expression of at least one gene identified in Table 1 and instructions for use.

In addition, the invention provides methods for active immunotherapy, such as, inducing an immune response in a subject by delivering the proteins, polypeptides and fragments thereof, as described herein, to the subject.

The polynucleotides and expression products having immunomodulatory activity are set forth in Table 1, below.

TABLE 1

| | | Unigene | LocusLink | GenBank References | |
|---|---|---|---|---|---|
| Description | Probe or Primer | Ref. | Id. | Name | |
| 206 aa 4 | GTGGTATGGC | Hs 25199 | 51537 | NM_016498.2 | HSPC242 |
| cysteine | SEQ ID NO:1 | | | NP_057582 | Hypothetical |
| | | | | | SEQ ID NOS 2 & 3 |
| 283 aa 5 | CTAATAAACT | Hs | 51108 | NM 057109 | CGI-81 Protein |
| cysteine | SEQ ID NO:4 | 279583 | | NP 057109 | SEQ ID NOS 5 & 6 |
| 228 aa 4 | GCTCCCTACG | Hs | 10148 | NM_005755.2 | EBV Induced |
| cysteine | SEQ ID NO:7 | 185705 | | NP_005746 | Gene 3 |
| | | | | | SEQ ID NOS 8 & 9 |
| 227 aa 5 | TGTATTTTGA | Hs.29882 | 10447 | NM_014888.1 | FAM3C Family |
| cysteines | SEQ ID NO:10 | | | NP_055703 | with sequence |
| predicted N- | | | | | similarity 3, |
| terminal | | | | | member C |
| signal peptide | | | | | SEQ ID NOS 11 & 12 |
| 86 aa 5 | GTCCCCCCAC | Hs | 7940 | NM_007161 | LST-1 |
| cysteine | SEQ ID NO:13 | 380427 | | NP 009092 | membrane |
| | | | | | receptor |
| | | | | | alternative |
| | | | | | spliced soluble |
| | | | | | form |
| | | | | | SEQ ID NOS 14 & 15 |
| 277 aa 8 | ACCTTGAAGT | Hs 29352 | 7130 | NM_007115.2 | TNFa induced |
| cysteine | SEQ ID NO:16 | | | NP 009046 | protein 6 |
| | | | | | SEQ ID NO:17 |
| 194 aa 9 | GGACCGAGGG | Hs 13885 | 84246 | NP 115662 | MGCS3O9 |
| cysteine | SEQ ID NO:18 | | | | Protein |
| | | | | | SEQ ID NOS 19 & 20 |
| 439 aa 11 | GCCGGCTTGA | Hs | 11026 | NM_006865.1 | ILT6 soluble |
| cysteine | SEQ ID NO:21 | 113277 | | NP 006856 | inhibitory |
| | | | | | receptor |
| | | | | | SEQ ID NOS 22 & 23 |
| 298 aa 6 | TTCACTGTAA | Hs | 4969 | NM_014057.2 | Osteoglycin- |
| cysteine | SEQ ID NO:24 | 109439 | | NP 054776 | induces ectopic |
| | | | | | bone formation |
| | | | | | SEQ ID NO:25 |
| 305 aa 7 | AATTCTGTAA | Hs | 84572 | NM_032520.2 | CAB56184 |
| Cysteine | SEQ ID NO:26 | 241575 | | NP 115909 | Hypothetical |
| | | | | | SEQ ID NOS 27 & 28 |
| 293 aa 9 | GTCTACAATT | Hs | 116150 | NM_138459 | Chromosome 6 |
| cysteine | SEQ ID NO:29 | 289008 | | NP 612468 | ORF 68 |
| | | | | | SEQ ID NOS 30 & 31 |
| 323 aa 6 | GGGCTCGGGG | Hs | None | NM_002975 | Stem cell growth |
| Cysteine | SEQ ID NO:32 | 105927 | | NP 002966 | factor |
| | | | | | SEQ ID NOS 33 & 34 |
| 270 aa 1 | CTGCTAACCC | Hs.170310 | 51816 | NM_016498.2 | CECR1 Cat eye |
| cysteine | SEQ ID NO:38 | | | NP_059120 | syndrome |
| predicted N- | | | | (Isoform a | chromosome |
| terminal signal | | | | 511 aa) | region, |
| peptide | | | | NM_016498.2 | candidate 1 |
| | | | | NP_803124 | SEQ ID NO: |
| | | | | (Isoform b | 39 |
| | | | | 270 aa) | SEQ ID NOS |
| | | | | | 40 & 41 |
| 173 aa 2 | AGCCTGCAGA | Hs.10927 | 56005 | XP_035638 | IL27w; |
| cysteines | SEQ ID NO:42 | | | | interleukin 27 |
| predicted N- | | | | | working |
| terminal signal | | | | | designation |
| peptide | | | | | SEQ ID NO: 43 |
| 142 aa 8 | AAAAGAAATG | Hs.41185 | 157869 | NM_153225 | FLJ40021 |
| cysteines | SEQ ID NO:44 | | | NP_694957 | Hypothetical |
| predicted N- | | | | | protein |
| terminal signal | | | | | SEQ ID NOS |
| peptide | | | | | 45 & 46 |
| 332 aa 7 | ATGACCTGAA | Hs.74050 | 2531 | NM 002035 | Follicular |
| cysteines | SEQ ID NO:47 | | | | lymphoma |
| | | | | | variant |
| | | | | | SEQ ID NOS |
| | | | | | 48 & 49 |
| 160 aa 5 | TTTAATACAT | Hs. | 84191 | NM 032231 | FLJ22875 |
| cysteines | SEQ ID NO:50 | 406548 | | | Hypothetical |
| | | | | | SEQ ID NOS |
| | | | | | 51 & 52 |
| 375 aa 8 | TGTAAACAAT | Hs. | 5157 | NM 006207 | Platelet |
| cysteines | SEQ ID NO:53 | 170040 | | | Derived |
| | | | | | Growth Factor |
| | | | | | Receptor Like |
| | | | | | SEQ ID NOS |
| | | | | | 54 & 55 |
| 194 aa 9 | GGACCGAGGG | Hs.13885 | 84246 | NM 032286 | MGC5309 |
| cysteines | SEQ ID NO:56 | | | | Hypothetical |
| | | | | | SEQ ID NOS |
| | | | | | 57 & 58 |
| 355 aa 22 | TCCAACCCAG | Hs. | 7478 | NM 031933 | WNT8A |
| cysteines | SEQ ID NO:59 | 302163 | | | SEQ ID NOS |
| | | | | | 60 & 61 |
| 248 aa 5 | ACTCGAATAT | Hs.95655 | 6398 | NM_003004.1 | SECTM1; |
| cysteines | SEQ ID NO:62 | | | NP_002995 | secreted and |
| predicted N- | | | | | transmembrane |
| terminal signal | | | | | 1; also known |
| peptide + | | | | | as K12 protein |
| transmembrane | | | | | precursor type |
| | | | | | 1a |
| | | | | | SEQ ID NOS |
| | | | | | 63 & 64 |

### MODES FOR CARRYING OUT THE INVENTION

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

Definitions

The practice of the present invention will employ, unless otherwise indicated, conventional techniques of immunology, molecular biology, microbiology, cell biology and recombinant DNA, which are within the skill of the art. See, e.g., Sambrook, Fritsch and Maniatis, MOLECULAR CLONING: A LABORATORY MANUAL, 2^{nd} edition (1989); CURRENT PROTOCOLS IN MOLECULAR BIOLOGY (F. M. Ausubel, et al. eds., (1987)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)).

As used herein, certain terms have the following defined meanings.

As used in the specification and claims, the singular form “a”, “an” and “the” include plural references unless the context clearly dictates otherwise. For example, the term “a cell” includes a plurality of cells, including mixtures thereof.

The terms “polynucleotide” and “oligonucleotide” are used interchangeably, and refer to a polymeric form of nucleotides of any length, either deoxyribonucleotides or ribonucleotides, or analogs thereof. Polynucleotides can have any three-dimensional structure, and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide can comprise modified nucleotides, such as methylated nucleotides and nucleotide analogs. If present, modifications to the nucleotide structure can be imparted before or after assembly of the polymer. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this invention that is a polynucleotide encompasses both the double-stranded form and each of two complementary single-stranded forms known or predicted to make up the double-stranded form.

A polynucleotide is composed of a specific sequence of four nucleotide bases: adenine (A); cytosine (C); guanine (G); thymine (T); and uracil (U) for guanine when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching.

A “gene” refers to a polynucleotide containing at least one open reading frame (ORF) that is capable of encoding a particular polypeptide or protein after being transcribed and translated. Any of the polynucleotides sequences described herein may be used to identify larger fragments or full-length coding sequences of the gene with which they are associated. Methods of isolating larger fragment sequences are known to those of skill in the art.

A “gene product” or alternatively a “gene expression product” refers to the amino acid (e.g., peptide or polypeptide) generated when a gene is transcribed and translated.

The term “polypeptide” is used interchangeably with the term “protein” and in its broadest sense refers to a compound of two or more subunit amino acids, amino acid analogs, or peptidomimetics. The subunits may be linked by peptide bonds. In another embodiment, the subunit may be linked by other bonds, e.g., ester, ether, etc. As used herein the term “amino acid” refers to either natural and/or unnatural or synthetic amino acids, including glycine and both the D or L optical isomers, and amino acid analogs and peptidomimetics. A peptide of three or more amino acids is commonly called an oligopeptide if the peptide chain is short. If the peptide chain is long, the peptide is commonly called a polypeptide or a protein.

“Under transcriptional control” is a term well understood in the art and indicates that transcription of a polynucleotide sequence, usually a DNA sequence, depends on its being operatively linked to an element which contributes to the initiation of, or promotes, transcription. “Operatively linked” refers to a juxtaposition wherein the elements are in an arrangement allowing them to function.

The following terms define the proteins and polynucleotides identified in Table 1, infra.

As used herein, the term “HSPC242 gene” refers to at least the ORF of a contiguous polynucleotide sequence and that encodes a protein or polypeptide having the biological activity as set forth herein. Sequence ID NO.: 3 is one example of an HSPC242 gene, and others are known in the art examples of which include, but are not limited to the sequences set forth under GenBank Accession No: NM_{—}016498.2 and the sequences that encode HSPC242 gene expression products as defined herein. See Zhang et al. (2000) Genome Res. 10(10):1546-1560. Also included within this definition are biologically equivalent sequences such as those sequences that code for the polypeptide of SEQ ID NO.: 2 and those having at least 90% or alternatively, at least 95% sequence homology to an exemplary sequence, such as SEQ ID NO.: 3, and as determined by percent identity sequence analysis run under default parameters. Also within this definition are biologically equivalent genes or polynucleotides that are identified by the ability to hybridize under conditions of high stringency to the minus strand. It may be desirable to use non-human genes, the polynucleotide sequences of which are known in the art, see for example, UniGene Cluster Hs 25199.

As used herein, the term “HSPC242 gene expression product, protein or polypeptide” includes the amino acid sequence of SEQ ID NO.: 2 (see Zhang et al. (2000) supra and GenBank Accession No. NP_{—}057582) as well as the amino acid sequences transcribed and translated from the HSPC242 genes identified above, without regard to the gene expression system, e.g., bacterial or other prokaryotic cell, yeast cell, mammalian cell such as a simian, bovine or human cell. The term includes isolated, naturally occurring polypeptides isolated from tissue samples as well as recombinantly produced proteins and polypeptides. The term also includes polypeptides having the amino acid sequences that are at least 90% or alternatively at least 95% homologous to SEQ ID NO.: 2 and which have the biological activity as set forth herein. Examples of homologous amino acid sequences include, but are not limited to polypeptides having the amino acid sequence of SEQ ID NO.: 2 or other HSPC242 gene expression product that has been modified by conservative amino acid substitutions.

Zhang et al. (2000) supra, reported isolation of the HSPC242 sequence from three hundred cDNAs containing putatively entire open reading frames (ORFs) obtained from CD34⁺ hematopoietic stem/progenitor cells (HSPCs), based on EST cataloging, clone sequencing, in silico cloning, and rapid amplification of cDNA ends (RACE).

As used herein, the term “IGSF6 (DORA) or “CGI-81” gene” refers to at least the ORF of a contiguous polynucleotide sequence and that encodes a protein or polypeptide having the biological activity as set forth herein. Sequence ID NO.: 6 is one example of an CGI-81 gene, and others are known in the art examples of which include, but are not limited to the sequences set forth under GenBank Accession No: NM_{—}016025.2 (see Bates et al. (2000) Immunogenetics 52(1-2):112-120) and the sequences that encode CGI-81 gene expression products as defined herein. Also included within this definition are biologically equivalent sequences such as those sequences that code for the polypeptide of SEQ ID NO.: 5 and those having at least 90% or alternatively, at least 95% sequence homology to an exemplary sequence, such as SEQ ID NO.: 6, and as determined by percent identity sequence analysis run under default parameters. Also within this definition are biologically equivalent genes or polynucleotides that are identified by the ability to hybridize under conditions of high stringency to the minus strand. It may be desirable to use non-human genes, the polynucleotide sequences of which are known in the art, see for example, UniGene Cluster Hs 279583.

As used herein, the term “CGI-81 gene expression product, protein or polypeptide” includes the amino acid sequence of SEQ ID NO.: 5 (see also GenBank Accession No. NP_{—}057582) as well as the amino acid sequences transcribed and translated from the CGI-81 genes identified above, without regard to the gene expression system, e.g., bacterial or other prokaryotic cell, yeast cell, mammalian cell such as a simian, bovine or human cell. The term includes isolated, naturally occurring polypeptides isolated from tissue samples as well as recombinantly produced proteins and polypeptides. The term also includes polypeptides having the amino acid sequences that are at least 90% or alternatively at least 95% homologous to SEQ ID NO.: 5 and which have the biological activity of this sequence as set forth herein. Examples of homologous amino acid sequences include, but are not limited to polypeptides having the amino acid sequence of SEQ ID NO.: 5 or other CGI-81 gene expression product that has been modified by conservative amino acid substitutions.

Bates et al. (2000) supra, report that IGSF6 (DORA), is a novel member of the immunoglobulin superfamily (IGSF) from human and rat expressed in dendritic and myeloid cells. Using a probe from the open reading frame of the rat cDNA, the authors isolated a cosmid which contains the entire mouse gene. By comparative analysis and reverse transcriptase polymerase chain reaction, the intron/exon structure and the mRNA of the mouse gene and, with respect to human BAC clones, the human gene, were defined by the authors. The genes span 10 kb (mouse) and 12 kb (human), with six exons arranged in a manner similar to other members of the IGSF. All intron/exon boundaries follow the GT-AG rule. The authors report that expression of the mouse IGSF6 gene is restricted to cells of the immune system, particularly macrophages. The human and mouse genes were localized to a locus associated with inflammatory bowel disease. The authors report that this gene is transcribed and processed, and is reported to contain homologues in Caenorhabditis elegans and prokaryotes, and is expressed in most organs in the mouse.

As used herein, the term “Epstein-Barr virus (EBV) Induced Gene 3” (“EBI3”) gene” refers to at least the ORF of a contiguous polynucleotide sequence and that encodes a protein or polypeptide having the biological activity as set forth herein. Sequence ID No.: 9 is one example of an EBI3 gene, and others are known in the art examples of which include, but are not limited to the sequence set forth under GenBank Accession No.: NM_{—}005755.2 and the sequences that encode EBV-3 gene expression products as defined herein. (See also Devergne, et al. (1996) PNAS 94(22):12041-12046 and (2000) J. Virol. 70(2):1143-1153). Also included within this definition are biologically equivalent sequences such as those sequences that code for the polypeptide of SEQ ID NO: 8 and those having at least 90% or alternatively, at least 95% sequence homology to an exemplary sequence, such as SEQ ID NO.: 9, and as determined by percent identity sequence analysis run under default parameters. Also within this definition are biologically equivalent genes or polynucleotides that are identified by the ability to hybridize under conditions of high stringency to the minus strand. It may be desirable to use non-human genes, the polynucleotide sequences of which are known in the art, see for example, UniGene Cluster Hs. 185705.

As used herein, the term “EBI3 gene expression product, protein or polypeptide” includes the amino acid sequence of SEQ ID NO.: 8 (see also GenBank Accession No. NP_{—}005746) as well as the amino acid sequences transcribed and translated from the EBI3 genes identified above, without regard to the gene expression system, e.g., bacterial or other prokaryotic cell, yeast cell, mammalian cell such as a simian, bovine or human cell. The term includes isolated, naturally occurring polypeptides isolated from tissue samples as well as recombinantly produced proteins and polypeptides. The term also includes polypeptides having the amino acid sequences that are at least 90% or alternatively at least 95% homologous to SEQ ID NO.: 8 and which have the biological activity as set forth herein herein. Examples of homologous amino acid sequences include, but are not limited to polypeptides have the amino acid sequence of SEQ ID NO.: 8 or other EBI3 gene expression product that has been modified by conservative amino acid substitutions.

Devergne (1996) supra and (2000) supra originally identified this gene and protein product by the induction of its expression in B lymphocytes by Epstein-Barr virus infection. The protein product is a secreted glycoprotein which is a member of the hematopoietion receptor family related to the p40 subunit of interleukin 12 (IL-12).

As used herein, the term “FAM3C family with sequence similarity 3, member C (“FAM3C”) gene” refers to at least the ORF of a contiguous polynucleotide sequence and that encodes a protein or polypeptide having the biological activity as set forth herein. Sequence ID NO.: 12 is one example of an FAM3C gene, and others are known in the art examples of which include, but are not limited to the sequences set forth under GenBank Accession NO.: NM_{—}014888.1 and the sequences that encode FAM3C gene expression products as defined herein. Also included within this definition are biologically equivalent sequences such as those sequences that code for the polypeptide of SEQ ID NO: 11 and those having at least 90% or alternatively, at least 95% sequence homology to an exemplary sequence, such as SEQ ID NO.: 11, and as determined by percent identity sequence analysis run under default parameters. Also within this definition are biologically equivalent genes or polynucleotides that are identified by the ability to hybridize under conditions of high stringency to the minus strand. It may be desirable to use non-human genes, the polynucleotide sequences of which are known in the art, see for example, UniGene Cluster Hs. 29882.

As used herein, the term “FAM3C gene expression product, protein or polypeptide” includes the amino acid sequence of SEQ ID NO.: 11 (see also GenBank Accession No.: NP_{—}055703.1) as well as the amino acid sequences transcribed and translated from the FAM3C genes identified above, without regard to the gene expression system, e.g., bacterial or other prokaryotic cell, yeast cell, mammalian cell such as a simian, bovine or human cell. The term includes isolated, naturally occurring polypeptides isolated from tissue samples as well as recombinantly produced proteins and polypeptides. The term also includes polypeptides having the amino acid sequences that are at least 90% or alternatively at least 95% homologous to SEQ ID NO.: 11 and which have the biological activity as described herein. Examples of homologous amino acid sequences include, but are not limited to polypeptides having the amino acid sequence of SEQ ID NO.: 11 or other FAM3C gene expression product that has been modified by conservative amino acid substitutions.

Zhu et al. (2002) Genomics 80(2):144-150, reported the identification and characterization of a novel cytokine-like gene family using structure-based methods to search for novel four-helix-bundle cytokines in genomics databases. Four genes were reported in this family, FAM3A, FAM3B, FAM3C, and FAM3D, each encoding a protein (224-235 amino acids) with a hydrophobic leader sequence. Northern analysis indicates that FAM3B is highly expressed in pancreas, FAM3D in placenta, and FAM3A and FAM3C in almost all tissues.

As used herein, the term leukocyte specific transcript 1 “(“LST-1”) gene” refers to at least the ORF of a contiguous polynucleotide sequence and that encodes a protein or polypeptide having the biological activity as set forth herein. Sequence ID NO.: 15 is one example of an LST-1 gene, and others are known in the art examples of which include, but are not limited to the sequences set forth under GenBank Accession No.: NM_{—}007161 and the sequences that encode LST-1 gene expression products as defined herein. (See Holzinger et al. (1995) Immunogenetics 42:315-322; Spies et al. (1989) Science 243:214-217.) Also included within this definition are biologically equivalent sequences such as those sequences that code for the polypeptide of SEQ ID NO: 14 and those having at least 90% or alternatively, at least 95% sequence homology to an exemplary sequence, such as SEQ ID NO.: 15, and as determined by percent identity sequence analysis run under default parameters. Also within this definition are biologically equivalent genes or polynucleotides that are identified by the ability to hybridize under conditions of high stringency to the minus strand. It may be desirable to use non-human genes, the polynucleotide sequences of which are known in the art, see for example, UniGene Cluster Hs 380427.

As used herein, the term “LST-1 gene expression product, protein or polypeptide” includes the amino acid sequence of SEQ ID NO.: 14 as well as the amino acid sequences transcribed and translated from the LST-1 genes identified above, without regard to the gene expression system, e.g., bacterial or other prokaryotic cell, yeast cell, mammalian cell such as a simian, bovine or human cell. The term includes isolated, naturally occurring polypeptides isolated from tissue samples as well as recombinantly produced proteins and polypeptides. The term also includes polypeptides having the amino acid sequences that are at least 90% or alternatively at least 95% homologous to SEQ ID NO.: 14 and which have the biological activity as described herein. Examples of homologous amino acid sequences include, but are not limited to polypeptides having the amino acid sequence of SEQ ID NO.: 14 or other LST-1S gene expression product that has been modified by conservative amino acid substitutions.

Holzinger et al. (1995) supra, reported that the LST-1 (LST-1) gene is constitutively expressed in leukocytes and dendritic cells, and it is characterized by extensive alternative splicing. The authors reported seven (7) different LST-1 splice variants in PBMC; thus, 14 LST-1 splice variants (LST-1/A-LST-1/N) have been detected in various cell types. The authors also reported that these isoforms code for transmembrane as well as soluble LST-1 proteins characterized by two alternative open reading frames at their 3′ end. The authors further reported that the presence of the transmembrane variant LST-1/C on the cell surface of the monocytic cell lines U937 and THP1. Recombinant expression of LST-1/C permitted its profound inhibitory effect on lymphocyte proliferation to be observed. In contrast, the alternative transmembrane variant LST-1/A, the extracellular domain of which shows no amino acid sequence homology to LST-1/C exerted a weaker but similar inhibitory effect on PBMC. The authors concluded that this data demonstrate the protein expression of LST-1 on the cell surface of mononuclear cells, and they show an inhibitory effect on lymphocyte proliferation of two LST-1 proteins although they have only a very short amino acid homology.

As used herein, the term “TNFαIP6 tumor necrosis factor, alpha-induced protein 6 gene” (“TNFα-IP6 gene”) refers to at least the ORF of a contiguous polynucleotide sequence and that encodes a protein or polypeptide having the biological activity as set forth herein. Sequences that encode Seq. ID NO.: 17 are examples of TNFα-IP6 genes, see, e.g., the sequences that encode the protein set forth under GenBank Accession No.: NM_{—}007115 and the sequences that encode TNFa-IP6 gene expression products as defined herein. Also included within this definition are biologically equivalent sequences such as those sequences that code for the polypeptide of SEQ ID NO: 17 and those having at least 90% or alternatively, at least 95% sequence homology to an exemplary sequence, such as GenBank Accession No.: NM_{—}007115, and as determined by percent identity sequence analysis run under default parameters. Also within this definition are biologically equivalent genes or polynucleotides that are identified by the ability to hybridize under conditions of high stringency to the minus strand. It may be desirable to use non-human genes, the polynucleotide sequences of which are known in the art, see for example, UniGene Cluster Hs 29352.

As used herein, the term “TNFα-IP6 gene expression product, protein or polypeptide” includes the amino acid sequence of SEQ ID NO.: 17 as well as the amino acid sequences transcribed and translated from the TNFα-IP6 genes identified above, without regard to the gene expression system, e.g., bacterial or other prokaryotic cell, yeast cell, mammalian cell such as a simian, bovine or human cell. The term includes isolated, naturally occurring polypeptides isolated from tissue samples as well as recombinantly produced proteins and polypeptides. The term also includes polypeptides having the amino acid sequences that are at least 90% or alternatively at least 95% homologous to SEQ ID NO.: 17 and which have the biological activity as described herein. Examples of homologous amino acid sequences include, but are not limited to polypeptides having the amino acid sequence of SEQ ID NO.: 17 or other TNFα-IP6 gene expression product that has been modified by conservative amino acid substitutions.

Lee et al. (1992) J. Cell Biol. 116(2):545-557, reported that the protein encoded by the TNFαIP6 gene is a secretory protein that contains a hyaluronan-binding domain, and thus is a member of the hyaluronan-binding protein family. The hyaluronan-binding domain is known to be involved in extracellular matrix stability and cell migration. This protein has been shown to form a stable complex with inter-alpha-inhibitor (1 alpha 1), and thus enhances the serine protease inhibitory activity of 1 alpha I, which is important in the protease network associated with inflammation. The authors also reported that expression of this gene can be induced by tumor necrosis factor alpha and interleukin-1.

As used herein, the term “MGC5309 gene” refers to at least the ORF of a contiguous polynucleotide sequence and that encodes a protein or polypeptide having the biological activity as set forth herein. Sequence ID NO.: 20 is one example of an MGC5309 gene, and others are known in the art examples of which include, but are not limited to the sequences that encode proteins set forth under GenBank Accession NO: NP_{—}115662 and the sequences that encode MGC5309 gene expression products as defined herein. Also included within this definition are biologically equivalent sequences such as those sequences that code for the polypeptide of SEQ ID NO: 19 and those having at least 90% or alternatively, at least 95% sequence homology to an exemplary sequence, such as SEQ ID NO.: 20, and as determined by percent identity sequence analysis run under default parameters. Also within this definition are biologically equivalent genes or polynucleotides that are identified by the ability to hybridize under conditions of high stringency to the minus strand. It may be desirable to use non-human genes, the polynucleotide sequences of which are known in the art, see for example, UniGene Cluster Hs 13885.

As used herein, the term “MGC5309 gene expression product, protein or polypeptide” includes the amino acid sequence of SEQ ID NO.: 19 as well as the amino acid sequences transcribed and translated from the MGC5309 genes identified above, without regard to the gene expression system, e.g., bacterial or other prokaryotic cell, yeast cell, mammalian cell such as a simian, bovine or human cell. The term includes isolated, naturally occurring polypeptides isolated from tissue samples as well as recombinantly produced proteins and polypeptides. The term also includes polypeptides having the amino acid sequences that are at least 90% or alternatively at least 95% homologous to SEQ ID NO.: 19 and which have the biological activity as described herein. Examples of homologous amino acid sequences include, but are not limited to polypeptides having the amino acid sequence of SEQ ID NO.: 19 or other MGC5309 gene expression product that has been modified by conservative amino acid substitutions.

As used herein, the term leukocyte immunoglobulin-like receptor (“ILT-6 gene”) refers to at least the ORF of a contiguous polynucleotide sequence and that encodes a protein or polypeptide having the biological activity as set forth herein. Sequence ID NO.: 23 is one example of an ILT-6 gene, and others are known in the art examples of which include, but are not limited to the sequences set forth under GenBank Accession NO: NM_{—}006865 and the sequences that encode ILT-6 gene expression products as defined herein. Also included within this definition are biologically equivalent sequences such as those sequences that code for the polypeptide of SEQ ID NO: 22 and those having at least 90% or alternatively, at least 95% sequence homology to an exemplary sequence, such as SEQ ID NO.: 23, and as determined by percent identity sequence analysis run under default parameters. Also within this definition are biologically equivalent genes or polynucleotides that are identified by the ability to hybridize under conditions of high stringency to the minus strand. It may be desirable to use non-human genes, the polynucleotide sequences of which are known in the art, see for example, UniGene Cluster Hs 113277.

As used herein, the term “ILT-6 gene expression product, protein or polypeptide” includes the amino acid sequence of SEQ ID NO.: 22 (see also GenBank Accession No. NP_{—}006856) as well as the amino acid sequences transcribed and translated from the ILT-6 genes identified above, without regard to the gene expression system, e.g., bacterial or other prokaryotic cell, yeast cell, mammalian cell such as a simian, bovine or human cell. The term includes isolated, naturally occurring polypeptides isolated from tissue samples as well as recombinantly produced proteins and polypeptides. The term also includes polypeptides having the amino acid sequences that are at least 90% or alternatively at least 95% homologous to SEQ ID NO.: 22 and which have the biological activity as described herein. Examples of homologous amino acid sequences include, but are not limited to polypeptides having the amino acid sequence of SEQ ID NO.: 22 or other ILT-6 gene expression product that has been modified by conservative amino acid substitutions.

Borges et al. (1997) J. Immunol. 159(11):5192-5196, reported that leukocyte Ig-like receptors (LIRs) are a family of immunoreceptors expressed on monocytes and B cells and at lower levels on dendritic cells and NK cells and that the amino acid sequences in the extracellular regions of eight of these receptors show between 63 and 84% identity to a LIR-1 sequence. LIRs contain either two or four Ig domains and fall into three classes: those with cytoplasmic domains containing two, three, or four immunoreceptor tyrosine-based inhibitory motif-like motifs; those with a short cytoplasmic domain and no immunoreceptor tyrosine-based inhibitory motif-like motifs; and those with no transmembrane domain represented by a single LIR molecule that is presumably secreted. The authors reported that the LIRs are structurally related to the human Fc(alpha)R and the killer inhibitory receptors and map to the same region of chromosome 19 as these genes.

As used herein, the term “Osteoglycin (osteoinductive factor) (“OGN”) gene” refers to at least the ORF of a contiguous polynucleotide sequence and that encodes a protein or polypeptide having the biological activity as set forth herein. Examples of OGN genes, known in the art, examples of which include, but are not limited to the sequences set forth under GenBank Accession No: NM 014057.2 and the sequences that encode OGN gene expression products as defined herein. Also included within this definition are biologically equivalent sequences such as those sequences that code for the polypeptide of SEQ ID NO: 25 and those having at least 90% or alternatively, at least 95% sequence homology to an exemplary sequence, such as that deposited under GenBank Accession No: NM_{—}014057.2, and as determined by percent identity sequence analysis run under default parameters. Also within this definition are biologically equivalent genes or polynucleotides that are identified by the ability to hybridize under conditions of high stringency to the minus strand. It may be desirable to use non-human genes, the polynucleotide sequences of which are known in the art, see for example, UniGene Cluster Hs 109439.

As used herein, the term “OGN gene expression product, protein or polypeptide” includes the amino acid sequence of SEQ ID NO.: 25 (e.g., see GenBank Accession No.: NP_{—}054476.1) as well as the amino acid sequences transcribed and translated from the OSC genes identified above, without regard to the gene expression system, e.g., bacterial or other prokaryotic cell, yeast cell, mammalian cell such as a simian, bovine or human cell. The term includes isolated, naturally occurring polypeptides isolated from tissue samples as well as recombinantly produced proteins and polypeptides. The term also includes polypeptides having the amino acid sequences that are at least 90% or alternatively at least 95% homologous to SEQ ID NO.: 25 and which have the biological activity as described herein. Examples of homologous amino acid sequences include, but are not limited to polypeptides having the amino acid sequence of SEQ ID NO.: 25 or other OGN gene expression product that has been modified by conservative amino acid substitutions.

Pellagata et al. (2000) Nature Genetics 25(1):91-95, report that OGN was highly expressed in the cornea.

As used herein, the term “CAB56184 gene” refers to at least the ORF of a contiguous polynucleotide sequence and that encodes a protein or polypeptide having the biological activity as set forth herein. The gene was isolated from chromosome 6. Sequence ID NO.: 28 is one example of an CAB56184 gene, and others are known in the art examples of which include, but are not limited to the sequence set forth under GenBank Accession No.: NM_{—}032520.2 and the sequences that encode CAB56184 gene expression products as defined herein. Also included within this definition are biologically equivalent sequences such as those sequences that code for the polypeptide of SEQ ID NO: 27 and those having at least 90% or alternatively, at least 95% sequence homology to an exemplary sequence, such as SEQ ID NO.: 28, and as determined by percent identity sequence analysis run under default parameters. Also within this definition are biologically equivalent genes or polynucleotides that are identified by the ability to hybridize under conditions of high stringency to the minus strand. It may be desirable to use non-human genes, the polynucleotide sequences of which are known in the art, see for example, UniGene Cluster Hs 241575.

As used herein, the term “CAB56184 gene expression product, protein or polypeptide” includes the amino acid sequence of SEQ ID NO.: 27 (see also GenBank Accession No. NP_{—}115909) as well as the amino acid sequences transcribed and translated from the CAB56184 genes identified above, without regard to the gene expression system, e.g., bacterial or other prokaryotic cell, yeast cell, mammalian cell such as a simian, bovine or human cell. The term includes isolated, naturally occurring polypeptides isolated from tissue samples as well as recombinantly produced proteins and polypeptides. The term also includes polypeptides having the amino acid sequences that are at least 90% or alternatively at least 95% homologous to SEQ ID NO.: 27 and which have the biological activity as described herein. Examples of homologous amino acid sequences include, but are not limited to polypeptides having the amino acid sequence of SEQ ID NO.: 27 or other CAB56184 gene expression product that has been modified by conservative amino acid substitutions.

As used herein, the term “Chromosome 6 ORF68 (“C6-68”) gene” refers to at least the ORF of a contiguous polynucleotide sequence and that encodes a protein or polypeptide having the biological activity as set forth herein. Sequence ID NO.: 31 is one example of an C6-68 gene, and others are known in the art examples of which include, but are not limited to the sequence set forth under GenBank Accession No: NM_{—}138459 and the sequences that encode C6-68 gene expression products as defined herein. Also included within this definition are biologically equivalent sequences such as those sequences that code for the polypeptide of SEQ ID NO: 30 and those having at least 90% or alternatively, at least 95% sequence homology to an exemplary sequence, such as SEQ ID NO.: 31, and as determined by percent identity sequence analysis run under default parameters. Also within this definition are biologically equivalent genes or polynucleotides that are identified by the ability to hybridize under conditions of high stringency to the minus strand. It may be desirable to use non-human genes, the polynucleotide sequences of which are known in the art, see for example, UniGene Cluster Hs 289008.

As used herein, the term “C6-68 gene expression product, protein or polypeptide” includes the amino acid sequence of SEQ ID NO.: 30 (see also GenBank Accession No.: NP_{—}612468) as well as the amino acid sequences transcribed and translated from the C6-68 genes identified above, without regard to the gene expression system, e.g., bacterial or other prokaryotic cell, yeast cell, mammalian cell such as a simian, bovine or human cell. The term includes isolated, naturally occurring polypeptides isolated from tissue samples as well as recombinantly produced proteins and polypeptides. The term also includes polypeptides having the amino acid sequences that are at least 90% or alternatively at least 95% homologous to SEQ ID NO.: 30 and which have the biological activity as set forth herein. Examples of homologous amino acid sequences include, but are not limited to polypeptides having the amino acid sequence of SEQ ID NO.: 30 or other C6-68 gene expression product that has been modified by conservative amino acid substitutions.

As used herein, the term “Stem Cell Growth Factor (“SCGF”) gene” refers to at least the ORF of a contiguous polynucleotide sequence that encodes a protein or polypeptide having the biological activity as set forth herein. Sequence ID NO.: 34 is one example of an SCGF gene, and others are known in the art examples of which include, but are not limited to the sequence set forth under GenBank Accession No: NM_{—}002975 and the sequences that encode SCGF gene expression products as defined herein. Also included within this definition are biologically equivalent sequences such as those sequences that code for the polypeptide of SEQ ID NO: 33 and those having at least 90% or alternatively, at least 95% sequence homology to an exemplary sequence, such as SEQ ID NO.: 34, and as determined by percent identity sequence analysis run under default parameters. Also within this definition are biologically equivalent genes or polynucleotides that are identified by the ability to hybridize under conditions of high stringency to the minus strand. It may be desirable to use non-human genes, the polynucleotide sequences of which are known in the art, see for example, UniGene Cluster Hs 105927.

As used herein, the term “SCGF gene expression product, protein or polypeptide” includes the amino acid sequence of SEQ ID NO.: 33 (see also the protein described under GenBank Accession No.: NP_{—}002966) as well as the amino acid sequences transcribed and translated from the SCGF genes identified above, without regard to the gene expression system, e.g., bacterial or other prokaryotic cell, yeast cell, mammalian cell such as a simian, bovine or human cell. The term includes isolated, naturally occurring polypeptides isolated from tissue samples as well as recombinantly produced proteins and polypeptides. The term also includes polypeptides having the amino acid sequences that are at least 90% or alternatively at least 95% homologous to SEQ ID NO.: 33 and which have the biological activity as described herein. Examples of homologous amino acid sequences include, but are not limited to polypeptides having the amino acid sequence of SEQ ID NO.: 33 or other SCGF gene expression product that has been modified by conservative amino acid substitutions.

Akin et al. (2002) Am. J. Clin. Pathol. 118(2):242-247, reported the presence of stem all factor in lesional mast cells isolated from bone marrow biopsies. Meade et al. (2002) Biochem. Pharm. 64(2):317-325, suggested that stem cell factor may play a role, within adenozine, in asthma.

As used herein, the term “HT036 gene” refers to at least the ORF of a contiguous polynucleotide sequence and that encodes a protein or polypeptide having the biological activity as set forth herein. Sequence ID NO.: 37 is one example of an HTO36 gene, and others are known in the art examples of which include, but are not limited to the sequences that encode the protein set forth under GenBank Accession No: NP_{—}112484 and the sequences that encode HTO36 gene expression products as defined herein. Also included within this definition are biologically equivalent sequences such as those sequences that code for the polypeptide of SEQ ID NO: 36 (see also GenBank Accession No. NM_{—}031207) and those having at least 90% or alternatively, at least 95% sequence homology to an exemplary sequence, such as SEQ ID NO.: 37, and as determined by percent identity sequence analysis run under default parameters. Also within this definition are biologically equivalent genes or polynucleotides that are identified by the ability to hybridize under conditions of high stringency to the minus strand. It may be desirable to use non-human genes, the polynucleotide sequences of which are known in the art, see for example, UniGene Cluster Hs 321669.

As used herein, the term “HTO36 gene expression product, protein or polypeptide” includes the amino acid sequence of SEQ ID NO.: 36 as well as the amino acid sequences transcribed and translated from the HTO36 genes identified above, without regard to the gene expression system, e.g., bacterial or other prokaryotic cell, yeast cell, mammalian cell such as a simian, bovine or human cell. The term includes isolated, naturally occurring polypeptides isolated from tissue samples as well as recombinantly produced proteins and polypeptides. The term also includes polypeptides having the amino acid sequences that are at least 90% or alternatively at least 95% homologous to SEQ ID NO.: 36 (see also GenBank Accession No. NP_{—}112484) and which have the biological activity as described herein. Examples of homologous amino acid sequences include, but are not limited to polypeptides having the amino acid sequence of SEQ ID NO.: 36 or other HTO36 gene expression product that has been modified by conservative amino acid substitutions.

As used herein, the term “cat eye syndrome chromosome region, candidate I” (“CECR1 gene”) refers to at least the ORF of a contiguous polynucleotide sequence and that encodes a protein or polypeptide having the biological activity as set forth herein. Sequence ID NO.: 41 (see also GenBank Accession No. NM_{—}177405.1) is one example of an CECR1 gene, and others are known in the art examples of which include, but are not limited to the sequences that encode the protein sequence set forth under GenBank Accession Nos: NP_{—}059120 and NP_{—}803124 and the sequences that encode CECR1 gene expression products as defined herein. Also included within this definition are biologically equivalent sequences such as those sequences that code for the polypeptide of SEQ ID NOS: 39 and 40 and those having at least 90% or alternatively, at least 95% sequence homology to an exemplary sequence, such as SEQ ID NO.: 41, and as determined by percent identity sequence analysis run under default parameters. Also within this definition are biologically equivalent genes or polynucleotides that are identified by the ability to hybridize under conditions of high stringency to the minus strand. It may be desirable to use non-human genes, the polynucleotide sequences of which are known in the art, see for example, UniGene Cluster Hs 170310.

As used herein, the term “CECR1 gene expression product, protein or polypeptide” includes the amino acid sequence of SEQ ID NOS.: 39 and 40 (see also proteins described under GenBank Accession Nos.: NP_{—}059120 and NP_{—}803124) as well as the amino acid sequences transcribed and translated from the CECR1 genes identified above, without regard to the gene expression system, e.g., bacterial or other prokaryotic cell, yeast cell, mammalian cell such as a simian, bovine or human cell. The term includes isolated, naturally occurring polypeptides isolated from tissue samples as well as recombinantly produced proteins and polypeptides. The term also includes polypeptides having the amino acid sequences that are at least 90% or alternatively at least 95% homologous to SEQ ID NOS.: 39 and 40 and which have the biological activity as described herein. Examples of homologous amino acid sequences include, but are not limited to polypeptides having the amino acid sequence of SEQ ID NOS.: 39 and 40 or other CECR1 gene expression product that has been modified by conservative amino acid substitutions.

Riazi et al. (2000) Genomics 64(3):277-285 reported that the gene is alternatively spliced and expressed in numerous tissues, with most abundant expression in human adult heart, lung, lymphoblasts, and placenta as well as fetal lung, liver, and kidney. The authors report that in situ hybridization of a human embryo shows specific expression in the outflow tract and atrium of the developing heart, the VII/VIII cranial nerve ganglion, and the notochord. The authors also reported that location of this gene in the CES critical region and its embryonic expression suggest that the overexpression of CECR1 may be responsible for at least some features of CES, particularly the heart defects.

Footz et al. (2001) Genome Res. 11(6): 1053-1070, sequenced and characterized a 1.1-Mb region of human chromosome 22g believed to be the dosage-sensitive gene responsible for cat eye syndrome (CES) as well as a putative 450-kb homologous region on mouse chromosome 6. The authors also reported that the gene encodes a member of a subfamily of the adenosine deaminase protein family. Two transcript variants encoding distinct isoforms were identified for this gene. Transcript variant (2) contains a distinct 5′ UTR and lacks an in-frame portion of the 5′ coding region, compared to variant 1. The resulting isoform (b) has a shorter N-terminus compared to isoform a.

As used herein, the term “interleukin 27 working designation” (“IL27W gene”) refers to at least the ORF of a contiguous polynucleotide sequence and that encodes a protein or polypeptide having the biological activity as set forth herein. The sequence deposited under GenBank Accession No: XP_{—}035638 is one example of an IL27W gene. Also included within this definition are biologically equivalent sequences such as those sequences that code for the polypeptide of SEQ ID NO: 43 and those having at least 90% or alternatively, at least 95% sequence homology the sequence deposited under GenBank Accession No: XP_{—}035638, and as determined by percent identity sequence analysis run under default parameters. Also within this definition are biologically equivalent genes or polynucleotides that are identified by the ability to hybridize under conditions of high stringency to the minus strand. It may be desirable to use non-human genes, the polynucleotide sequences of which are known in the art, see for example, UniGene Cluster Hs 10927.

As used herein, the term “IL27W gene expression product, protein or polypeptide” includes the amino acid sequence of SEQ ID NO.: 43 as well as the amino acid sequences transcribed and translated from the IL27W genes identified above, without regard to the gene expression system, e.g., bacterial or other prokaryotic cell, yeast cell, mammalian cell such as a simian, bovine or human cell. The term includes isolated, naturally occurring polypeptides isolated from tissue samples as well as recombinantly produced proteins and polypeptides. The term also includes polypeptides having the amino acid sequences that are at least 90% or alternatively at least 95% homologous to SEQ ID NO.: 43 and which have the biological activity as described herein. Examples of homologous amino acid sequences include, but are not limited to polypeptides having the amino acid sequence of SEQ ID NO.: 43 or other IL27W gene expression product that has been modified by conservative amino acid substitutions.

As used herein, the term “FLJ40021 gene” refers to at least the ORF of a contiguous polynucleotide sequence and that encodes a protein or polypeptide having the biological activity as set forth herein. Sequence ID NO.: 46 is one example of an FLJ40021 gene, and others are known in the art examples of which include, but are not limited to the sequence set forth under GenBank Accession No: NM_{—}153225 and the sequences that encode FLJ40021 gene expression products as defined herein (see, e.g. GenBank Accession No. NP_{—}694957). Also included within this definition are biologically equivalent sequences such as those sequences that code for the polypeptide of SEQ ID NO: 45 and those having at least 90% or alternatively, at least 95% sequence homology to an exemplary sequence, such as SEQ ID NO.: 46, and as determined by percent identity sequence analysis run under default parameters. Also within this definition are biologically equivalent genes or polynucleotides that are identified by the ability to hybridize under conditions of high stringency to the minus strand. It may be desirable to use non-human genes, the polynucleotide sequences of which are known in the art, see for example, UniGene Cluster Hs 41185.

As used herein, the term “FLJ40021 gene expression product, protein or polypeptide” includes the amino acid sequence of SEQ ID NO.: 45 as well as the amino acid sequences transcribed and translated from the FLJ40021 genes identified above, without regard to the gene expression system, e.g., bacterial or other prokaryotic cell, yeast cell, mammalian cell such as a simian, bovine or human cell. The term includes isolated, naturally occurring polypeptides isolated from tissue samples as well as recombinantly produced proteins and polypeptides. The term also includes polypeptides having the amino acid sequences that are at least 90% or alternatively at least 95% homologous to SEQ ID NO.: 45 and which have the biological activity as described herein. Examples of homologous amino acid sequences include, but are not limited to polypeptides having the amino acid sequence of SEQ ID NO.: 45 or other FLJ40021 gene expression product that has been modified by conservative amino acid substitutions.

As used herein, the term “Follicular lymphoma variant (“FLV”) gene” refers to at least the ORF of a contiguous polynucleotide sequence and that encodes a protein or polypeptide having the biological activity as set forth herein. Sequence ID NO.: 49 is one example of an FLV gene, and others are known in the art examples of which include, but are not limited to the sequence set forth under GenBank Accession No: NM_{—}002035 and the sequences that encode FLV gene expression products as defined herein. Also included within this definition are biologically equivalent sequences such as those sequences that code for the polypeptide of SEQ ID NO: 48 and those having at least 90% or alternatively, at least 95% sequence homology to an exemplary sequence, such as SEQ ID NO.: 49, and as determined by percent identity sequence analysis run under default parameters. Also within this definition are biologically equivalent genes or polynucleotides that are identified by the ability to hybridize under conditions of high stringency to the minus strand. It may be desirable to use non-human genes, the polynucleotide sequences of which are known in the art, see for example, UniGene Cluster Hs 74050.

As used herein, the term “FLV gene expression product, protein or polypeptide” includes the amino acid sequence of SEQ ID NO.: 48 as well as the amino acid sequences transcribed and translated from the FLV genes identified above, without regard to the gene expression system, e.g., bacterial or other prokaryotic cell, yeast cell, mammalian cell such as a simian, bovine or human cell. The expression product is reported to be a secreted protein weakly expressed in hematopoietic tissue. It has shown a high rate of transcription in some T-cell malignancies and in phytohemagglutinin-stimulated lymphocytes. The term includes isolated, naturally occurring polypeptides isolated from tissue samples as well as recombinantly produced proteins and polypeptides. The term also includes polypeptides having the amino acid sequences that are at least 90% or alternatively at least 95% homologous to SEQ ID NO.: 48 and which have the biological activity as described herein. Examples of homologous amino acid sequences include, but are not limited to polypeptides having the amino acid sequence of SEQ ID NO.: 48 or other FLV gene expression product that has been modified by conservative amino acid substitutions.

As used herein, the term “FLJ22875 (“FLJ22875”) gene” refers to at least the ORF of a contiguous polynucleotide sequence and that encodes a protein or polypeptide having the biological activity as set forth herein. Sequence ID NO.: 52 is one example of an FLJ gene, and others are known in the art examples of which include, but are not limited to the sequences set forth under GenBank Accession No: NM_{—}032231 and the sequences that encode FLJ22875 gene expression products as defined herein. Also included within this definition are biologically equivalent sequences such as those sequences that code for the polypeptide of SEQ ID NO: 51 and those having at least 90% or alternatively, at least 95% sequence homology to an exemplary sequence, such as SEQ ID NO.: 52, and as determined by percent identity sequence analysis run under default parameters. Also within this definition are biologically equivalent genes or polynucleotides that are identified by the ability to hybridize under conditions of high stringency to the minus strand. It may be desirable to use non-human genes, the polynucleotide sequences of which are known in the art, see for example, UniGene Cluster Hs 406548.

As used herein, the term “FLJ22875 gene expression product, protein or polypeptide” includes the amino acid sequence of SEQ ID NO.: 51 as well as the amino acid sequences transcribed and translated from the FLJ22875 genes identified above, without regard to the gene expression system, e.g., bacterial or other prokaryotic cell, yeast cell, mammalian cell such as a simian, bovine or human cell. The term includes isolated, naturally occurring polypeptides isolated from tissue samples as well as recombinantly produced proteins and polypeptides. The term also includes polypeptides having the amino acid sequences that are at least 90% or alternatively at least 95% homologous to SEQ ID NO.: 51 and which have the biological activity as described herein. Examples of homologous amino acid sequences include, but are not limited to polypeptides having the amino acid sequence of SEQ ID NO.: 51 or other FLJ22875 gene expression product that has been modified by conservative amino acid substitutions.

As used herein, the term “platelet-derived growth factor receptor-like gene” “PDGF gene” refers to at least the ORF of a contiguous polynucleotide sequence and that encodes a protein or polypeptide having the biological activity as set forth herein. Sequence ID NO.: 55 is one example of an PDGF gene, and others are known in the art examples of which include, but are not limited to the sequence set forth under GenBank Accession No: NM_{—}006207 and the sequences that encode PDGF gene expression products as defined herein. Also included within this definition are biologically equivalent sequences such as those sequences that code for the polypeptide of SEQ ID NO: 54 and those having at least 90% or alternatively, at least 95% sequence homology to an exemplary sequence, such as SEQ ID NO.: 55, and as determined by percent identity sequence analysis run under default parameters. Also within this definition are biologically equivalent genes or polynucleotides that are identified by the ability to hybridize under conditions of high stringency to the minus strand. It may be desirable to use non-human genes, the polynucleotide sequences of which are known in the art, see for example, UniGene Cluster Hs 170040.

As used herein, the term “PDGF gene expression product, protein or polypeptide” includes the amino acid sequence of SEQ ID NO.: 54 as well as the amino acid sequences transcribed and translated from the PDGF genes identified above, without regard to the gene expression system, e.g., bacterial or other prokaryotic cell, yeast cell, mammalian cell such as a simian, bovine or human cell. The term includes isolated, naturally occurring polypeptides isolated from tissue samples as well as recombinantly produced proteins and polypeptides. The term also includes polypeptides having the amino acid sequences that are at least 90% or alternatively at least 95% homologous to SEQ ID NO.: 54 and which have the biological activity as shown herein. Examples of homologous amino acid sequences include, but are not limited to polypeptides having the amino acid sequence of SEQ ID NO.: 54 or other PDGF gene expression product that has been modified by conservative amino acid substitutions.

This gene is reported to encode a protein with significant sequence similarity to the ligand binding domain of platelet-derived growth factor receptor beta. Mutations in this gene, or deletion of a chromosomal segment containing this gene, are associated with sporadic hepatocellular carcinomas, colorectal cancers, and non-small cell lung cancers.

As used herein, the term “WNT8A gene” refers to at least the ORF of a contiguous polynucleotide sequence and that encodes a protein or polypeptide having the biological activity as set forth herein. Sequence ID NO.: 61 is one example of an WNT8A gene, and others are known in the art examples of which include, but are not limited to the sequence set forth under GenBank Accession No.: NM_{—}031933 and the sequences that encode WNT8A gene expression products as defined herein. Also included within this definition are biologically equivalent sequences such as those sequences that code for the polypeptide of SEQ ID NO: 60 and those having at least 90% or alternatively, at least 95% sequence homology to an exemplary sequence, such as SEQ ID NO.: 61, and as determined by percent identity sequence analysis run under default parameters. Also within this definition are biologically equivalent genes or polynucleotides that are identified by the ability to hybridize under conditions of high stringency to the minus strand. It may be desirable to use non-human genes, the polynucleotide sequences of which are known in the art, see for example, UniGene Cluster Hs 302163.

As used herein, the term “WNT8A gene expression product, protein or polypeptide” includes the amino acid sequence of SEQ ID NO.: 60 as well as the amino acid sequences transcribed and translated from the WNT8A genes identified above, without regard to the gene expression system, e.g., bacterial or other prokaryotic cell, yeast cell, mammalian cell such as a simian, bovine or human cell. The term includes isolated, naturally occurring polypeptides isolated from tissue samples as well as recombinantly produced proteins and polypeptides. The term also includes polypeptides having the amino acid sequences that are at least 90% or alternatively at least 95% homologous to SEQ ID NO.: 60 and which have the biological activity as described herein. Examples of homologous amino acid sequences include, but are not limited to polypeptides having the amino acid sequence of SEQ ID NO.: 60 or other WNT8A gene expression product that has been modified by conservative amino acid substitutions.

Saitoh and Katoh (2001) Int. J. Oncol. 19:123-127, searched human genome draft sequences for homologs of the Xenopus wnt8 gene and identified WNT8A. The authors assembled a full-length WNT8A cDNA sequence. The authors predicted that the 6 exons of the WNT8A gene encode a 351-amino acid protein with an N-terminal signal peptide, 3 N-linked glycosylation sites, and residues conserved among members of the WNT family. The WNT8A protein shares 63.2% sequence identity with human WNT8B and homology with the mouse Wnt8d protein.

As used herein, the term “secreted and transmembrane 1 gene” “SECTM1 gene” refers to at least the ORF of a contiguous polynucleotide sequence and that encodes a protein or polypeptide having the biological activity as set forth herein. Sequence ID NO.: 64 is one example of an SECTM1 gene, and others are known in the art examples of which include, but are not limited to the sequence set forth under GenBank Accession No.: NM_{—}003004.1 and the sequences that encode SECTM1 gene expression products as defined herein. Also included within this definition are biologically equivalent sequences such as those sequences that code for the polypeptide of SEQ ID NO: 63 and those having at least 90% or alternatively, at least 95% sequence homology to an exemplary sequence, such as SEQ ID NO.: 64, and as determined by percent identity sequence analysis run under default parameters. Also within this definition are biologically equivalent genes or polynucleotides that are identified by the ability to hybridize under conditions of high stringency to the minus strand. It may be desirable to use non-human genes, the polynucleotide sequences of which are known in the art, see for example, UniGene Cluster Hs 95655.

As used herein, the term “SECTM1 gene expression product, protein or polypeptide” includes the amino acid sequence of SEQ ID NO.: 63 as well as the amino acid sequences transcribed and translated from the SECTM1 genes identified above, without regard to the gene expression system, e.g., bacterial or other prokaryotic cell, yeast cell, mammalian cell such as a simian, bovine or human cell. The term includes isolated, naturally occurring polypeptides isolated from tissue samples as well as recombinantly produced proteins and polypeptides. The term also includes polypeptides having the amino acid sequences that are at least 90% or alternatively at least 95% homologous to SEQ ID NO.: 63 and which have the biological activity as described herein. Examples of homologous amino acid sequences include, but are not limited to polypeptides having the amino acid sequence of SEQ ID NO.: 63 or other SECTM1 gene expression product that has been modified by conservative amino acid substitutions.

Slentz-Kesler et al. (1998) Genomics 47(3):327-340, reported that the investigation of a DNase-hypersensitive site upstream of the CD7 gene on chromosome 17q25 led to the discovery of a novel human gene designated K12 (SECTM1). This gene spans approximately 14 kb and encodes a 1.8-kb mRNA detected at the highest levels in peripheral blood leukocytes and breast cancer cell lines. The open reading frame predicts a 248-amino-acid protein with the hydropathic characteristics of a type 1a membrane protein. Western blots showed that the K12 protein exists as a cluster of bands around 27 kDa, and extractions using nonionic detergents or high pH conditions demonstrate that it behaves as an integral membrane protein. Immunofluorescence localization studies reveal that K12 is not detectable on the cell surface, but instead is found in a perinuclear Golgi-like pattern and colocalizes with a well-known Golgi marker. The authors report that in addition, an approximately 20-kDa soluble form of the K12 protein derived from the N-terminal domain is specifically secreted by cells into the culture medium. Immunohistochemical analysis of peripheral blood cells shows that K12 is found in leukocytes of the myeloid lineage, with the strongest staining observed in granulocytes and no detectable expression in lymphocytes.

As used herein, the term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

The term “isolated” means separated from constituents, cellular and otherwise, in which the polynucleotide, peptide, polypeptide, protein, antibody, or fragments thereof, are normally associated with in nature. In one aspect of this invention, an isolated polynucleotide is separated from the 3′ and 5′ contiguous nucleotides with which it is normally associated with in its native or natural environment, e.g., on the chromosome. As is apparent to those of skill in the art, a non-naturally occurring polynucleotide, peptide, polypeptide, protein, antibody, or fragments thereof, does not require “isolation” to distinguish it from its naturally occurring counterpart. In addition, a “concentrated”, “separated” or “diluted” polynucleotide, peptide, polypeptide, protein, antibody, or fragments thereof, is distinguishable from its naturally occurring counterpart in that the concentration or number of molecules per volume is greater than “concentrated” or less than “separated” than that of its naturally occurring counterpart. A polynucleotide, peptide, polypeptide, protein, antibody, or fragments thereof, which differs from the naturally occurring counterpart in its primary sequence or for example, by its glycosylation pattern, need not be present in its isolated form since it is distinguishable from its naturally occurring counterpart by its primary sequence, or alternatively, by another characteristic such as glycosylation pattern. Thus, a non-naturally occurring polynucleotide is provided as a separate embodiment from the isolated naturally occurring polynucleotide. A protein produced in a bacterial cell is provided as a separate embodiment from the naturally occurring protein isolated from a eukaryotic cell in which it is produced in nature.

“Gene delivery,” “gene transfer,” and the like as used herein, are terms referring to the introduction of an exogenous polynucleotide (sometimes referred to as a “transgene”) into a host cell, irrespective of the method used for the introduction. Such methods include a variety of well-known techniques such as vector-mediated gene transfer (by, e.g., viral infection/transfection, or various other protein-based or lipid-based gene delivery complexes) as well as techniques facilitating the delivery of “naked” polynucleotides (such as electroporation, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are known in the art to be capable of mediating transfer of genes to mammalian cells.

A “gene delivery vehicle” is defined as any molecule that can carry inserted polynucleotides into a host cell. Examples of gene delivery vehicles are liposomes, biocompatible polymers, including natural polymers and synthetic polymers; lipoproteins; polypeptides; polysaccharides; lipopolysaccharides; artificial viral envelopes; recombinant yeast cells, metal particles; and bacteria, or viruses, such as baculovirus, adenovirus and retrovirus, bacteriophage, cosmid, plasmid, fungal vectors and other recombination vehicles typically used in the art which have been described for expression in a variety of eukaryotic and prokaryotic hosts, and may be used for gene therapy as well as for simple protein expression.

A “viral vector” is defined as a recombinantly produced virus or viral particle that comprises a polynucleotide to be delivered into a host cell, either in vivo, ex vivo or in vitro. Examples of viral vectors include retroviral vectors, adenovirus vectors, adeno-associated virus vectors, alphavirus vectors and the like. Alphavirus vectors, such as Semliki Forest virus-based vectors and Sindbis virus-based vectors, have also been developed for use in gene therapy and immunotherapy. See, Schlesinger and Dubensky (1999) Curr. Opin. Biotechnol. 5:434-439 and Ying et al. (1999) Nat. Med. 5(7):823-827. In aspects where gene transfer is mediated by a retroviral vector, a vector construct refers to the polynucleotide comprising the retroviral genome or part thereof, and a therapeutic gene. As used herein, “retroviral mediated gene transfer” or “retroviral transduction” carries the same meaning and refers to the process by which a gene or nucleic acid sequences are stably transferred into the host cell by virtue of the virus entering the cell and integrating its genome into the host cell genome. The virus can enter the host cell via its normal mechanism of infection or be modified such that it binds to a different host cell surface receptor or ligand to enter the cell. As used herein, retroviral vector refers to a viral particle capable of introducing exogenous nucleic acid into a cell through a viral or viral-like entry mechanism.

Retroviruses carry their genetic information in the form of RNA; however, once the virus infects a cell, the RNA is reverse-transcribed into the DNA form which integrates into the genomic DNA of the infected cell. The integrated DNA form is called a provirus.

In aspects where gene transfer is mediated by a DNA viral vector, such as an adenovirus (Ad) or adeno-associated virus (AAV), a vector construct refers to the polynucleotide comprising the viral genome or part thereof, and a transgene. Adenoviruses (Ads) are a relatively well characterized, homogenous group of viruses, including over 50 serotypes. See, e.g., WO 95/27071. Ads are easy to grow and do not require integration into the host cell genome. Recombinant Ad derived vectors, particularly those that reduce the potential for recombination and generation of wild-type virus, have also been constructed. See, WO 95/00655 and WO 95/11984. Wild-type AAV has high infectivity and specificity integrating into the host cell's genome. See, Hermonat and Muzyczka (1984) Proc. Natl. Acad. Sci. USA 81:6466-6470 and Lebkowski et al. (1988) Mol. Cell. Biol. 8:3988-3996.

Vectors that contain both a promoter and a cloning site into which a polynucleotide can be operatively linked are known in the art. Such vectors are capable of transcribing RNA in vitro or in vivo, and are commercially available from sources such as Stratagene (La Jolla, Calif.) and Promega Biotech (Madison, Wis.). In order to optimize expression and/or in vitro transcription, it may be necessary to remove, add or alter 5′ and/or 3′ untranslated portions of the clones to eliminate extra, potential inappropriate alternative translation initiation codons or other sequences that may interfere with or reduce expression, either at the level of transcription or translation. Alternatively, consensus ribosome binding sites can be inserted immediately 5′ of the start codon to enhance expression.

Gene delivery vehicles also include several non-viral vectors, including DNA/liposome complexes, recombinant yeast cells, and targeted viral protein-DNA complexes. Liposomes that also comprise a targeting antibody or fragment thereof can be used in the methods of this invention. To enhance delivery to a cell, the nucleic acid or proteins of this invention can be conjugated to antibodies or binding fragments thereof which bind cell surface antigens, e.g., TCR, CD3 or CD4.

A “probe” when used in the context of polynucleotide manipulation refers to an oligonucleotide that is provided as a reagent to detect a target potentially present in a sample of interest by hybridizing with the target. Usually, a probe will comprise a label or a means by which a label can be attached, either before or subsequent to the hybridization reaction. Suitable labels include, but are not limited to radioisotopes, fluorochromes, chemiluminescent compounds, dyes, and proteins, including enzymes.

A “primer” is a short polynucleotide, generally with a free 3′-OH group that binds to a target or “template” potentially present in a sample of interest by hybridizing with the target, and thereafter promoting polymerization of a polynucleotide complementary to the target. A “polymerase chain reaction” (“PCR”) is a reaction in which replicate copies are made of a target polynucleotide using a “pair of primers” or a “set of primers” consisting of an “upstream” and a “downstream” primer, and a catalyst of polymerization, such as a DNA polymerase, and typically a thermally-stable polymerase enzyme. Methods for PCR are known in the art, and taught, for example in “PCR: A PRACTICAL APPROACH” (M. MacPherson et al., IRL Press at Oxford University Press (1991)). All processes of producing replicate copies of a polynucleotide, such as PCR or gene cloning, are collectively referred to herein as “replication.” A primer can also be used as a probe in hybridization reactions, such as Southern or Northern blot analyses. Sambrook et al., supra.

An expression “database” denotes a set of stored data that represent a collection of sequences, which in turn represent a collection of biological reference materials.

The term “cDNAs” refers to complementary DNA that is mRNA molecules present in a cell or organism made into cDNA with an enzyme such as reverse transcriptase. A “cDNA library” is a collection of all of the mRNA molecules present in a cell or organism, all turned into cDNA molecules with the enzyme reverse transcriptase, then inserted into “vectors” (other DNA molecules that can continue to replicate after addition of foreign DNA). Exemplary vectors for libraries include bacteriophage (also known as “phage”), viruses that infect bacteria, for example, lambda phage. The library can then be probed for the specific cDNA (and thus mRNA) of interest.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell. “Differentially expressed” as applied to a gene, refers to the differential production of the mRNA transcribed and/or translated from the gene or the protein product encoded by the gene. A differentially expressed gene may be overexpressed or underexpressed as compared to the expression level of a normal or control cell. In one aspect, it refers to a differential that is 2.5 times, preferably 5 times, or preferably 10 times higher or lower than the expression level detected in a control sample. The term “differentially expressed” also refers to nucleotide sequences in a cell or tissue which are expressed where silent in a control cell or not expressed where expressed in a control cell.

As used herein, “solid phase support” or “solid support”, used interchangeably, is not limited to a specific type of support. Rather a large number of supports are available and are known to one of ordinary skill in the art. Solid phase supports include silica gels, resins, derivatized plastic films, glass beads, cotton, plastic beads, alumina gels, microarrays and chips. As used herein, “solid support” also includes synthetic antigen-presenting matrices, cells, and liposomes. A suitable solid phase support may be selected on the basis of desired end use and suitability for various protocols. For example, for peptide synthesis, solid phase support may refer to resins such as polystyrene (e.g., PAM-resin obtained from Bachem Inc., Peninsula Laboratories, etc.), POLYHIPE® resin (obtained from Aminotech, Canada), polyamide resin (obtained from Peninsula Laboratories), polystyrene resin grafted with polyethylene glycol (TentaGel®, Rapp Polymere, Tubingen, Germany) or polydimethylacrylamide resin (obtained from Milligen/Biosearch, Calif.).

A polynucleotide also can be attached to a solid support for use in high throughput screening assays. PCT WO 97/10365, for example, discloses the construction of high density oligonucleotide chips. See also, U.S. Pat. Nos. 5,405,783; 5,412,087; and 5,445,934. Using this method, the probes are synthesized on a derivatized glass surface also known as chip arrays. Photoprotected nucleoside phosphoramidites are coupled to the glass surface, selectively deprotected by photolysis through a photolithographic mask, and reacted with a second protected nucleoside phosphoramidite. The coupling/deprotection process is repeated until the desired probe is complete.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Hybridization reactions can be performed under conditions of different “stringency”. In general, a low stringency hybridization reaction is carried out at about 40° C. in 10×SSC or a solution of equivalent ionic strength/temperature. A moderate stringency hybridization is typically performed at about 50° C. in 6×SSC, and a high stringency hybridization reaction is generally performed at about 60° C. in 1×SSC.

When hybridization occurs in an antiparallel configuration between two single-stranded polynucleotides, the reaction is called “annealing” and those polynucleotides are described as “complementary”. A double-stranded polynucleotide can be “complementary” or “homologous” to another polynucleotide, if hybridization can occur between one of the strands of the first polynucleotide and the second. “Complementarity” or “homology” (the degree that one polynucleotide is complementary with another) is quantifiable in terms of the proportion of bases in opposing strands that are expected to form hydrogen bonding with each other, according to generally accepted base-pairing rules.

A polynucleotide or polynucleotide region (or a polypeptide or polypeptide region) has a certain percentage (for example, 80%, 85%, 90% or 95%) of “sequence identity” to another sequence means that, when aligned, that percentage of bases (or amino acids) are the same in comparing the two sequences. This alignment and the percent homology or sequence identity can be determined using software programs known in the art, for example those described in CURRENT PROTOCOLS IN MOLECULAR BIOLOGY (F. M. Ausubel et al., eds., 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: http://www.ncbi.nim.nih.gov/cgi-bin/BLAST.

Hyperplasia is a form of controlled cell proliferation involving an increase in cell number in a tissue or organ, without significant alteration in structure or function. Metaplasia is a form of controlled cell growth in which one type of fully differentiated cell substitutes for another type of differentiated cell. Metaplasia can occur in epithelial or connective tissue cells. Atypical metaplasia involves a somewhat disorderly metaplastic epithelium.

As used herein, the terms “pathological cells”, “neoplasia”, “tumor”, “tumor cells”, “cancer” and “cancer cells”, (used interchangeably) refer to cells which exhibit relatively autonomous growth, so that they exhibit an aberrant growth phenotype characterized by a significant loss of control of cell proliferation (i.e., de-regulated cell division). Pathological cells can be malignant or benign. A metastatic cell or tissue means that the cell can invade and destroy neighboring body structures.

“Suppressing” tumor growth indicates a growth state that is curtailed when compared to growth without contact with educated, antigen-specific immune effector cells described herein. Tumor cell growth can be assessed by any means known in the art, including, but not limited to, measuring tumor size, determining whether tumor cells are proliferating using a ³H-thymidine incorporation assay, or counting tumor cells. “Suppressing” tumor cell growth means any or all of the following states: slowing, delaying, and stopping tumor growth, as well as tumor shrinkage.

As used herein a second polynucleotide “corresponds to” another (a first) polynucleotide if it is related to the first polynucleotide by any of the following relationships:
1) The second polynucleotide comprises the first polynucleotide and the second polynucleotide encodes a gene product.
2) The second polynucleotide is 5′ or 3′ to the first polynucleotide in cDNA, RNA, genomic DNA, or fragment of any of these polynucleotides. For example, a second polynucleotide may be a fragment of a gene that includes the first and second polynucleotides. The first and second polynucleotides are related in that they are components of the gene coding for a gene product, such as a protein or antibody. However, it is not necessary that the second polynucleotide comprises or overlaps with the first polynucleotide to be encompassed within the definition of “corresponding to” as used herein. For example, the first polynucleotide may be a fragment of a 3′ untranslated region of the second polynucleotide, for example a promoter sequence. The first and second polynucleotide may be fragment of a gene coding for a gene product. The second polynucleotide may be an exon of the gene while the first polynucleotide may be an intron of the gene.
3) The second polynucleotide is the complement of the first polynucleotide.

The “genotype” of a cell refers to the genetic makeup of the cell and/or its gene expression profile. Modulation of the genotype of a cell can be achieved by introducing additional DNA or RNA either as episomes or as an integral part of the chromosomal DNA of the recipient cell. The genotype can also be modulated by altering the expression level, e.g. mRNA abundance, of a particular gene using agents that regulate gene expression.

A “database” denotes a set of stored data which represent a collection of sequences including nucleotide and peptide sequences, which in turn represent a collection of biological reference materials.

The term “sequence motif” refers to a pattern present in a group of molecules. For instance, in one embodiment, the present invention provides for identification of a sequence motif among peptides. In this embodiment, a typical pattern may be identified by characteristic amino acid residues, such as hydrophobic, hydrophilic, basic, acidic, and the like.

The terms “major histocompatibility complex” or “MHC” refers to a complex of genes encoding cell-surface molecules that are required for antigen presentation to T cells and for rapid graft rejection. In humans, the MHC complex is also known as the HLA complex. The proteins encoded by the MHC complex are known as “MHC molecules” and are classified into Class I and Class II MHC molecules. Class I MHC molecules include membrane heterodimeric proteins made up of an α chain encoded in the MHC associated noncovalently with β2-microglobulin. Class I MHC molecules are expressed by nearly all nucleated cells and have been shown to function in antigen presentation to CD8+ T cells. Class I molecules include HLA-A, -B, and -C in humans. Class I molecules generally bind peptides 8-10 amino acids in length. Class II MHC molecules also include membrane heterodimeric proteins consisting of noncovalently associated α and β chains. Class II MHC are known to participate in antigen presentation to CD4+ T cells and, in humans, include HLA-DP, -DQ, and DR. Class II molecules generally bind peptides 12-20 amino acid residues in length. The term “MHC restriction” refers to a characteristic of T cells that permits them to recognize antigen only after it is processed and the resulting antigenic peptides are displayed in association with either a self Class I or Class II MHC molecule. Methods of identifying and comparing MHC are known in the art and are described in Allen et al. (1994) Human Imm. 40:25-32; Santamaria et al. (1993) Human Imm. 37:39-50 and Hurley et al. (1997) Tissue Antigens 50:401-415.

The term “antigen-presenting matrix,” as used herein, intends a molecule or molecules which can present antigen in such a way that the antigen can be bound by a T-cell antigen receptor on the surface of a T cell. An antigen-presenting matrix can be on the surface of an antigen-presenting cell (APC), on a vesicle preparation of an APC, or can be in the form of a synthetic matrix on a solid support such as a bead or a plate. An example of a synthetic antigen-presenting matrix is purified MHC Class I molecules complexed to β2-microglobulin, or purified MHC Class II molecules, or functional portions thereof, attached to a solid support.

The term “antigen presenting cell,” as used herein, intends any cell which presents on its surface an antigen in association with a major histocompatibility complex molecule, or portion thereof, or, alternatively, one or more non-classical MHC molecules, or a portion thereof. Examples of suitable APCs are discussed in detail below and include, but are not limited to, whole cells such as macrophages, dendritic cells, B cells, hybrid APCs, and foster antigen presenting cells. Methods of making hybrid APCs have been described. See, for example, International Patent Application No. WO 98/46785; and WO 95/16775.

Dendritic cells (DCs) are potent antigen-presenting cells. It has been shown that DCs provide all the signals required for T cell activation and proliferation. These signals can be categorized into two types. The first type, which gives specificity to the immune response, is mediated through interaction between the T-cell receptor/CD3 (“TCR/CD3”) complex and an antigenic peptide presented by a major histocompatibility complex (“MHC”) Class I or II protein on the surface of APCs. This interaction is necessary, but not sufficient, for T cell activation to occur. In fact, without the second type of signals, the first type of signals can result in T cell anergy. The second type of signals, called co-stimulatory signals, is neither antigen-specific nor MHC-restricted, and can lead to a full proliferation response of T cells and induction of T cell effector functions in the presence of the first type of signals. As used herein, “dendritic cell” is to include, but not be limited to a pulsed dendritic cell, a foster cell or a dendritic cell hybrid.

“Co-stimulatory molecules” are involved in the interaction between receptor-ligand pairs expressed on the surface of antigen presenting cells and T cells. Research accumulated over the past several years has demonstrated convincingly that resting T cells require at least two signals for induction of cytokine gene expression and proliferation (Schwartz (1990) Science 248:1349-1356; Jenkins (1992) Immunol. Today 13:69-73). One signal, the one that confers specificity, can be produced by interaction of the TCR/CD3 complex with an appropriate MHC/peptide complex. The second signal is not antigen specific and is termed the “co-stimulatory” signal. This signal was originally defined as an activity provided by bone-marrow-derived accessory cells such as macrophages and dendritic cells, the so called “professional” APCs. Several molecules have been shown to enhance co-stimulatory activity. These are heat stable antigen (HSA) (Liu et al. (1992) J. Exp. Med. 175:437-445), chondroitin sulfate-modified MHC invariant chain (Ii-CS) (Naujokas et al. (1993) Cell 74:257-268), intracellular adhesion molecule 1 (ICAM-1) (Van Seventer (1990) J. Immunol. 144:4579-4586), B7-1, and B7-2/B70 (Schwartz (1992) Cell 71:1065-1068). These molecules each appear to assist co-stimulation by interacting with their cognate ligands on the T cells. Co-stimulatory molecules mediate co-stimulatory signal(s) which are necessary, under normal physiological conditions, to achieve full activation of naïve T cells. One exemplary receptor-ligand pair is the B7 co-stimulatory molecule on the surface of APCs and its counter-receptor CD28 or CTLA-4 on T cells (Freeman et al. (1993) Science 262:909-911; Young et al. (1992) J. Clin. Invest. 90: 229; Nabavi et al. (1992) Nature 360:266-268). Other important co-stimulatory molecules are CD40, CD54, CD80, CD86. The term “co-stimulatory molecule” encompasses any single molecule or combination of molecules which, when acting together with a peptide/MHC complex bound by a TCR on the surface of a T cell, provides a co-stimulatory effect which achieves activation of the T cell that binds the peptide. The term thus encompasses B7, or other co-stimulatory molecule(s) on an antigen-presenting matrix such as an APC, fragments thereof (alone, complexed with another molecule(s), or as part of a fusion protein) which, together with peptide/MHC complex, binds to a cognate ligand and results in activation of the T cell when the TCR on the surface of the T cell specifically binds the peptide. Co-stimulatory molecules are commercially available from a variety of sources, including, for example, Beckman Coulter. It is intended, although not always explicitly stated, that molecules having similar biological activity as wild-type or purified co-stimulatory molecules (e.g., recombinantly produced or muteins thereof) are intended to be used within the spirit and scope of the invention.

As used herein, “solid phase support” or “solid support,” used interchangeably, is not limited to a specific type of support. Rather a large number of supports are available and are known to one of ordinary skill in the art. Solid phase supports include silica gels, resins, derivatized plastic films, glass beads, cotton, plastic beads, alumina gels. As used herein, “solid support” also includes synthetic antigen-presenting matrices, cells, and liposomes. A suitable solid phase support may be selected on the basis of desired end use and suitability for various protocols. For example, for peptide synthesis, solid phase support may refer to resins such as polystyrene (e.g., PAM-resin obtained from Bachem Inc., Peninsula Laboratories, etc.), POLYHIPE® resin (obtained from Aminotech, Canada), polyamide resin (obtained from Peninsula Laboratories), polystyrene resin grafted with polyethylene glycol (TentaGel®, Rapp Polymere, Tubingen, Germany) or polydimethylacrylamide resin (obtained from Milligen/Biosearch, Calif.).

The term “immunomodulatory agent,” as used herein, is a molecule, a macromolecular complex, or a cell that modulates an immune response. The term “modulate an immune response” includes inducing (increasing, eliciting) an immune response; and reducing (suppressing) an immune response. An immunomodulatory method (or protocol) is one that modulates an immune response in a subject.

The term “immune effector cells” refers to cells capable of binding an antigen or which mediate an immune response. These cells include, but are not limited to, T cells, B cells, monocytes, macrophages, NK cells and cytotoxic T lymphocytes (CTLs), for example CTL lines, CTL clones, and CTLs from tumor, inflammatory, or other infiltrates. Certain diseased tissue expresses specific antigens and CTLs specific for these antigens have been identified. For example, approximately 80% of melanomas express the antigen known as gp100.

The term “immune effector molecule,” as used herein, refers to molecules capable of antigen-specific binding, and includes antibodies, T cell antigen receptors, and MHC Class I and Class II molecules.

A “naïve” immune effector cell is an immune effector cell that has never been exposed to an antigen.

As used herein, the term “educated, antigen-specific immune effector cell,” is an immune effector cell as defined above, which has encountered antigen and which is specific for that antigen. An educated, antigen-specific immune effector cell may be activated upon binding antigen. “Activated” implies that the cell is no longer in Go phase, and begins to produce cytokines characteristic of the cell type. For example, activated CD4+ T cells secrete IL-2 and have a higher number of high affinity IL-2 receptors on their cell surfaces relative to resting CD4+ T cells.

As used herein, the term “a disease or condition related to a population of CD4⁺ or CD8⁺ T cells” is one which can be related to a population of CD4⁺ or CD8⁺ T cells, such that these cells are primarily responsible for the pathogenesis of the disease; it is also one in which the presence of CD4⁺ or CD8⁺ T cells is an indicia of a disease state; it is also one in which the presence of a population CD4⁺ or CD8⁺ T cells is not the primary cause of the disease, but which plays a key role in the pathogenesis of the disease; it is also one in which a population of CD4⁺ or CD8⁺ T cells mediates an undesired rejection of a foreign antigen. Examples of a condition related to a population of CD4⁺ or CD8⁺ T cells include, but are not limited to, autoimmune disorders, graft rejection, immunoregulatory disorders, and anaphylactic disorders.

As used herein, the terms “pathological cells,” “neoplasia,” “tumor,” “tumor cells,” “cancer” and “cancer cells,” (used interchangeably) refer to cells which exhibit relatively autonomous growth, so that they exhibit an aberrant growth phenotype characterized by a significant loss of control of cell proliferation (i.e., de-regulated cell division). Pathological cells can be malignant or benign.

“Suppressing” tumor growth indicates a growth state that is curtailed when compared to growth without contact with educated, antigen-specific immune effector cells described herein. Tumor cell growth can be assessed by any means known in the art, including, but not limited to, measuring tumor size, determining whether tumor cells are proliferating using a ³H-thymidine incorporation assay, or counting tumor cells. “Suppressing” tumor cell growth means any or all of the following states: slowing, delaying, and stopping tumor growth, as well as tumor shrinkage.

The term “culturing” refers to the in vitro propagation of cells or organisms on or in media of various kinds. It is understood that the descendants of a cell grown in culture may not be completely identical (morphologically, genetically, or phenotypically) to the parent cell. By “expanded” is meant any proliferation or division of cells.

As used herein, the term “cytokine” refers to any one of the numerous factors that exert a variety of effects on cells, for example, inducing growth or proliferation. Non-limiting examples of cytokines which may be used alone or in combination in the practice of the present invention include, interleukin-2 (IL-2), stem cell factor (SCF), interleukin-3 (IL-3), interleukin-6 (IL-6), interleukin-12 (IL-12), G-CSF, granulocyte macrophage-colony stimulating factor (GM-CSF), interleukin-1 alpha (IL-1a), interleukin-11 (IL-11), MIP-1α, leukemia inhibitory factor (LIF), c-kit ligand, thrombopoietin (TPO) and flt3 ligand. The present invention also includes culture conditions in which one or more cytokine is specifically excluded from the medium. Cytokines are commercially available from several vendors such as, for example, Genzyme (Framingham, Mass.), Genentech (South San Francisco, Calif.), Amgen (Thousand Oaks, Calif.), R&D Systems and Immunex (Seattle, Wash.). It is intended, although not always explicitly stated, that molecules having similar biological activity as wild-type or purified cytokines (e.g., recombinantly produced or muteins thereof) are intended to be used within the spirit and scope of the invention.

A “composition” is intended to mean a combination of active agent and another compound or composition, inert (for example, a detectable agent or label) or active, such as an adjuvant.

A “pharmaceutical composition” is intended to include the combination of an active agent with a carrier, inert or active, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin, REMINGTON'S PHARM. SCI., 15th Ed. (Mack Publ. Co., Easton (1975)).

An “effective amount” is an amount sufficient to effect beneficial or desired results such as prevention or treatment. An effective amount can be administered in one or more administrations, applications or dosages.

A “subject,” “individual” or “patient” is used interchangeably herein, which refers to a vertebrate, preferably a mammal, more preferably a human. Mammals include, but are not limited to, murines, simians, humans, farm animals, sport animals, and pets.

A “control” is an alternative subject or sample used in an experiment for comparison purpose. A control can be “positive” or “negative”. For example, where the purpose of the experiment is to determine a correlation of an altered expression level of a gene with a particular type of cancer, it is generally preferable to use a positive control (a subject or a sample from a subject, carrying such alteration and exhibiting syndromes characteristic of that disease), and a negative control (a subject or a sample from a subject lacking the altered expression and clinical syndrome of that disease).

Diagnostic Methods

As noted above, this invention provides various methods for aiding in the diagnosis of the state of a cell or tissue that is characterized by differential expression of a protein identified in Table 1. The methods are particularly useful for aiding in the diagnosis of disorders relating to the immune response, e.g., cancers, autoimmune diseases and viral infections.

When referring to cancerous disorders, the pathological state of a cell or tissue generally is determined by noting whether the growth of the cell is not governed by the usual limitation of normal growth. For the purposes of this invention, the term also is to include genotypic changes that occur prior to detection of this growth in the form of a tumor and are causative of these phenotypic changes. The phenotypic changes associated with the pathological state of a cell (a set of in vitro characteristics associated with a tumorigenic ability in vivo) include a more rounded cell morphology, looser substratum attachment, loss of contact inhibition, loss of anchorage dependence, release of proteases such as plasminogen activator, increased sugar transport, decreased serum requirement, expression of fetal antigens and the like. (See, Luria et al. (1978) GENERAL VIROLOGY, 3^{d} edition, 436-446 (John Wiley & Sons, New York)).

Accordingly, one embodiment is a method of diagnosing the condition of a cell or tissue by screening for the presence of a differentially expressed gene isolated from a sample. In one aspect, the gene is expressed more in a pathological cell or tissue (e.g., autoimmune disorders), and is selected from the proteins identified in Table 1. Detection can be by any appropriate method, including for example, detecting the quantity of mRNA transcribed from the gene, or the quantity of cDNA produced from the reverse transcription of the mRNA transcribed from the gene, or the quantity of the polypeptide or protein encoded by the gene. Probes for each of these methods are provided in Table 1. These methods can be performed on a sample by sample basis or modified for high throughput analysis. Additionally, databases containing quantitative full or partial transcripts or protein sequences isolated from a cell sample can be searched and analyzed for the presence and amount of transcript or expressed gene product. In one aspect, the database contains at least one of the sequences shown in Table 1.

For the purpose of illustration only, gene expression is determined by noting the amount (if any, e.g., altered) expression of the gene in the test system at the level of an mRNA transcribed from at least one gene identified in Table 1. In a separate embodiment, augmentation of the level of the polypeptide or protein encoded by the gene is indicative of the presence of the pathological condition of the cell or tissue. For example, an enhanced or increase in the expression level of a gene identified in Table 1 may indicate infection or alternatively, an over-stimulated immune response that occurs, for example, in certain autoimmune disorders. In yet a further embodiment, a decrease in the level of polypeptide or protein encoded by the gene is indicative of the pathological condition, e.g., cancer. The method can be used for aiding in the diagnosis of cancer by detecting a genotype that is correlated with a phenotype characteristic of primary tumor cells. Thus, by detecting this genotype prior to tumor growth, one can predict a predisposition to cancer and/or provide early diagnosis and treatment.

Cell or tissue samples used for this invention encompass body fluid, solid tissue samples, tissue cultures or cells derived there from and the progeny thereof, and sections or smears prepared from any of these sources, or any other samples that may contain a cell having a gene described herein. In one embodiment, the sample comprises cells prepared from a subject's tissue.

In assaying for an alteration in mRNA level, nucleic acid contained in the aforementioned samples is first extracted according to standard methods in the art. For instance, mRNA can be isolated using various lytic enzymes or chemical solutions according to the procedures set forth in Sambrook et al. (1989) supra, or extracted by nucleic-acid-binding resins following the accompanying instructions provided by manufactures. The mRNA of a proto-oncogene of interest contained in the extracted nucleic acid sample is then detected by hybridization (e.g., Northern blot analysis) and/or amplification procedures according to methods widely known in the art or based on the methods exemplified herein.

Nucleic acid molecules having at least 10 nucleotides and exhibiting sequence complementarity or homology to at least one gene identified in Table 1 find utility as hybridization probes. It is known in the art that a “perfectly matched” probe is not needed for a specific hybridization. Minor changes in probe sequence achieved by substitution, deletion or insertion of a small number of bases do not affect the hybridization specificity. In general, as much as 20% base-pair mismatch (when optimally aligned) can be tolerated. Preferably, a probe useful for detecting mRNA is at least about 80% identical to the homologous region of comparable size contained in the genes or polynucleotides identified in Table 1 identified sequences, which have the Locus Link numbers identified in Table 1. In one aspect, the probe is 85% identical to the corresponding gene sequence after alignment of the homologous region, or alternatively, it exhibits 90% identity. Additional probes can be derived from sequences for the genes identified by the Locus Link Nos. provided in Table 1, or to a homologous region of comparable size contained in the previously identified sequences, which have the Locus Link Nos. identified in Table 1. These probes can be used in radioassays (e.g., Southern and Northern blot analysis) to detect, prognose, diagnose or monitor various pathological states resulting from differential expression of a gene of interest. The total size of fragment, as well as the size of the complementary stretches, will depend on the intended use or application of the particular nucleic acid segment. Smaller fragments derived from the known sequences will generally find use in hybridization embodiments, wherein the length of the complementary region may be varied, such as between about 10 and about 100 nucleotides, or even full length according to the complementary sequences one wishes to detect.

In one aspect, nucleotide probes having complementary sequences over stretches greater than about 10 nucleotides in length are used, so as to increase stability and selectivity of the hybrid, and thereby improving the specificity of particular hybrid molecules obtained. Alternatively, one can design nucleic acid molecules having gene-complementary stretches of more than about 25 or alternatively more than about 50 nucleotides in length, or even longer where desired. Such fragments may be readily prepared by, for example, directly synthesizing the fragment by chemical means, by application of nucleic acid reproduction technology, such as the PCR™ technology with two priming oligonucleotides as described in U.S. Pat. No. 4,603,102 or by introducing selected sequences into recombinant vectors for recombinant production. In one aspect, a probe is about 50 to about 75, nucleotides or alternatively, about 50 to about 100 nucleotides in length.

In certain embodiments, it will be advantageous to employ nucleic acid sequences as described herein in combination with an appropriate means, such as a label, for detecting hybridization and therefore complementary sequences. A wide variety of appropriate indicator means are known in the art, including fluorescent, radioactive, enzymatic or other ligands, such as avidin/biotin, which are capable of giving a detectable signal. One can employ a fluorescent label or an enzyme tag, such as urease, alkaline phosphatase or peroxidase, instead of radioactive or other environmental undesirable reagents. In the case of enzyme tags, calorimetric indicator substrates are known which can be employed to provide a means visible to the human eye or spectrophotometrically, to identify specific hybridization with complementary nucleic acid-containing samples.

Hybridization reactions can be performed under conditions of different “stringency”. Relevant conditions include temperature, ionic strength, time of incubation, the presence of additional solutes in the reaction mixture such as formamide, and the washing procedure. Higher stringency conditions are those conditions, such as higher temperature and lower sodium ion concentration, which require higher minimum complementarity between hybridizing elements for a stable hybridization complex to form. Conditions that increase the stringency of a hybridization reaction are widely known and published in the art. See, for example, Sambrook et al. (1989) supra.

The nucleotide probes of the present invention can also be used as primers and detection of genes or gene transcripts that are differentially expressed in certain body tissues. Additionally, a primer useful for detecting the aforementioned differentially expressed mRNA is at least about 80% identical to the homologous region of comparable size contained in the previously identified sequences, which have the Locus Link Nos. numbers identified in Table 1. For the purpose of this invention, amplification means any method employing a primer-dependent polymerase capable of replicating a target sequence with reasonable fidelity. Amplification may be carried out by natural or recombinant DNA-polymerases such as T7 DNA polymerase, Klenow fragment of E. coli DNA polymerase, and reverse transcriptase.

A known amplification method is PCR, MacPherson et al., PCR: A PRACTICAL APPROACH, (IRL Press at Oxford University Press (1991)). However, PCR conditions used for each application reaction are empirically determined. A number of parameters influence the success of a reaction. Among them are annealing temperature and time, extension time, Mg²⁺ ATP concentration, pH, and the relative concentration of primers, templates, and deoxyribonucleotides.

After amplification, the resulting DNA fragments can be detected by agarose gel electrophoresis followed by visualization with ethidium bromide staining and ultraviolet illumination. A specific amplification of differentially expressed genes of interest can be verified by demonstrating that the amplified DNA fragment has the predicted size, exhibits the predicated restriction digestion pattern, and/or hybridizes to the correct cloned DNA sequence.

The probes also can be attached to a solid support for use in high throughput screening assays using methods known in the art. PCT WO 97/10365 and U.S. Pat. Nos. 5,405,783; 5,412,087 and 5,445,934; for example, disclose the construction of high density oligonucleotide chips which can contain one or more of the sequences disclosed herein. Using the methods disclosed in U.S. Pat. Nos. 5,405,783; 5,412,087 and 5,445,934; the probes of this invention are synthesized on a derivatized glass surface. Photoprotected nucleoside phosphoramidites are coupled to the glass surface, selectively deprotected by photolysis through a photolithographic mask, and reacted with a second protected nucleoside phosphoramidite. The coupling/deprotection process is repeated until the desired probe is complete.

The expression level of a gene can also be determined through exposure of a nucleic acid sample to a probe-modified chip. Extracted nucleic acid is labeled, for example, with a fluorescent tag, preferably during an amplification step. Hybridization of the labeled sample is performed at an appropriate stringency level. The degree of probe-nucleic acid hybridization is quantitatively measured using a detection device, such as a confocal microscope. See U.S. Pat. Nos. 5,578,832 and 5,631,734. The obtained measurement is directly correlated with gene expression level.

The probes and high density oligonucleotide probe arrays also provide an effective means of monitoring expression of the genes identified in Table 1. They are also useful to screen for compositions that upregulate or downregulate the expression of the genes identified in Table 1.

In another embodiment, the methods of this invention are used to monitor expression of the genes identified in Table 1 which specifically hybridize to the probes of this invention in response to defined stimuli, such as an exposure of a cell or subject to a drug.

In one embodiment, the hybridized nucleic acids are detected by detecting one or more labels attached to the sample nucleic acids. The labels may be incorporated by any of a number of means known to those of skill in the art. However, in one aspect, the label is simultaneously incorporated during the amplification step in the preparation of the sample nucleic acid. Thus, for example, polymerase chain reaction (PCR) with labeled primers or labeled nucleotides will provide a labeled amplification product. In a separate embodiment, transcription amplification, as described above, using a labeled nucleotide (e.g., fluorescein-labeled UTP and/or CTP) incorporates a label in to the transcribed nucleic acids.

Alternatively, a label may be added directly to the original nucleic acid sample (e.g., mRNA, polyA, mRNA, cDNA, etc.) or to the amplification product after the amplification is completed. Means of attaching labels to nucleic acids are known to those of skill in the art and include, for example nick translation or end-labeling (e.g., with a labeled RNA) by kinasing of the nucleic acid and subsequent attachment (ligation) of a nucleic acid linker joining the sample nucleic acid to a label (e.g., a fluorophore).

Detectable labels suitable for use in the present invention include any composition detectable by spectroscopic, photochemical, biochemical, immunochemical, electrical, optical or chemical means. Useful labels in the present invention include biotin for staining with labeled streptavidin conjugate, magnetic beads (e.g., Dynabeads™), fluorescent dyes (e.g., fluorescein, Texas red, rhodamine, green fluorescent protein, and the like), radiolabels (e.g., 3H, ¹²⁵I, ³⁵S, ¹⁴C, or ³²P) enzymes (e.g., horse radish peroxidase, alkaline phosphatase and others commonly used in an ELISA), and calorimetric labels such as colloidal gold or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241.

Means of detecting such labels are known to those of skill in the art. Thus, for example, radiolabels may be detected using photographic film or scintillation counters, fluorescent markers may be detected using a photodetector to detect emitted light. Enzymatic labels are typically detected by providing the enzyme with a substrate and detecting the reaction product produced by the action of the enzyme on the substrate, and calorimetric labels are detected by simply visualizing the colored label.

As described in more detail in WO 97/10365, the label may be added to the target (sample) nucleic acid(s) prior to, or after the hybridization. These are detectable labels that are directly attached to or incorporated into the target (sample) nucleic acid prior to hybridization. In contrast, “indirect labels” are joined to the hybrid duplex after hybridization. Often, the indirect label is attached to a binding moiety that has been attached to the target nucleic acid prior to the hybridization. Thus, for example, the target nucleic acid may be biotinylated before the hybridization. After hybridization, an avidin-conjugated fluorophore will bind the biotin bearing hybrid duplexes providing a label that is easily detected. For a detailed review of methods of labeling nucleic acids and detecting labeled hybridized nucleic acids see LABORATORY TECHNIQUES IN BIOCHEMISTRY AND MOLECULAR BIOLOGY, Vol. 24: Hybridization with Nucleic Acid Probes, P. Tijssen, ed. Elsevier, N.Y. (1993).

The nucleic acid sample also may be modified prior to hybridization to the high density probe array in order to reduce sample complexity thereby decreasing background signal and improving sensitivity of the measurement using methods known in the art, e.g., the methods disclosed in WO 97/10365.

Results from the chip assay are typically analyzed using a computer software program. See, for example, EP 0717 113 A2 and WO 95/20681. The hybridization data is read into the program, which calculates the expression level of the targeted gene(s) i.e., the genes identified in Table 1. This figure is compared against existing data sets of gene expression levels for diseased and healthy individuals. A correlation between the obtained data and that of a set of diseased individuals indicates the onset of a disease in the subject patient.

Also within the scope of this application is a data base useful for the detection of pathological tissue comprising one or more of the sequences (or parts thereof) of the genes listed Table 1.

These polynucleotide sequences are stored in a digital storage medium such that a data processing system for standardized representation of the genes that correlate to a pathological condition. The data processing system is useful to analyze gene expression between two cells by first selecting a cell suspected of being of a pathological phenotype or genotype and then isolating polynucleotides from the cell or tissue. The isolated polynucleotides are then sequenced. The sequences from the sample are compared with the sequence(s) present in the database using homology search techniques described above. In one aspect, greater than 90% is selected, or alternatively greater than 95% is selected, or alternatively greater than or equal to 97% sequence identity is selected, between the test sequence and at least one sequence identified in Table 1 or its complement, is a positive indication that the polynucleotide has been isolated from a cell or tissue as defined above.

Alternatively, one can compare a sample against a database. Briefly, multiple RNAs are isolated from cell or tissue samples using methods known in the art and described for example, in Sambrook et al. (1989) supra. Optionally, the gene transcripts can be converted to cDNA. A sampling of the gene transcripts are subjected to sequence-specific analysis and quantified. These gene transcript sequence abundances are compared against reference database sequence abundances including normal data sets for diseased and healthy patients. The patient has the disease(s) with which the patient's data set most closely correlates which includes the expression of the transcripts identified herein.

Differential expression of the genes of interest can also be determined by examining the protein product. A variety of techniques are available in the art for protein analysis. They include but are not limited to radioimmunoassays, ELISA (enzyme linked immunoradiometric assays), “sandwich” immunoassays, immunoradiometric assays, in situ immunoassays (using e.g., colloidal gold, enzyme or radioisotope labels), western blot analysis, immunoprecipitation assays, immunofluorescent assays, and PAGE-SDS. One means to determine protein level involves (a) providing a biological sample containing polypeptides; and (b) measuring the amount of any immunospecific binding that occurs between an antibody reactive to the expression product of a gene of interest and a component in the sample, in which the amount of immunospecific binding indicates the level of the expressed proteins.

Antibodies that specifically recognize and bind to the protein products of these genes are required for these immunoassays. These may be purchased from commercial vendors or generated and screened using methods known in the art. See Harlow and Lane (1988) supra. and Sambrook et al. (1989) supra. Alternatively, polyclonal or monoclonal antibodies that specifically recognize and bind the protein product of a gene of interest can be made and isolated using known methods.

In diagnosing pathologies characterized by a differential expression of genes, one typically conducts a comparative analysis of the subject and appropriate controls. Preferably, a diagnostic test includes a control sample derived from a subject (hereinafter “positive control”), that exhibits the predicted change in expression of a gene of interest, e.g., at a level of at least 2.5 fold and clinical characteristics of the pathology of interest. Alternatively, a diagnosis also includes a control sample derived from a subject (hereinafter “negative control”), that lacks the clinical characteristics of the pathological state and whose expression level of the gene at question is within a normal range. A positive correlation between the subject and the positive control with respect to the identified alterations indicates the presence of or a predisposition to said disease. A lack of correlation between the subject and the negative control confirms the diagnosis.

There are various methods available in the art for quantifying mRNA or protein level from a cell sample and indeed, any method that can quantify these levels is encompassed by this invention. For example, determination of the mRNA level of the aforementioned genes may involve, in one aspect, measuring the amount of mRNA in a sample isolated from the cell by hybridization or quantitative amplification using at least one oligonucleotide probe that is complementary to the mRNA. Determination of the aforementioned gene products requires measuring the amount of immunospecific binding that occurs between an antibody reactive to the gene product of a gene identified in Table 1. To detect and quantify the immunospecific binding, or signals generated during hybridization or amplification procedures, digital image analysis systems including but not limited to those that detect radioactivity of the probes or chemiluminescence can be employed.

Screening Assays

The present invention also provides a screen for identifying leads, drugs, therapeutic biologics, and methods for reversing the pathological condition of the subject by modifying the immune response. In one aspect, the screen identifies lead compounds or biological agents which are useful for the treatment of malignancy, hyperplasia metaplasia, autoimmune diseases and viral infections characterized by differential expression of a gene identified in Table 1.

Thus, to practice the method in vitro, suitable cell cultures or tissue cultures are first provided. The cell can be a cultured cell or a genetically modified cell which differentially expresses a gene associated with a pathological cell e.g., at least one gene identified in Table 1. Alternatively, the cells can be from a tissue biopsy. The cells are cultured under conditions (temperature, growth or culture medium and gas (CO₂)) and for an appropriate amount of time to attain exponential proliferation without density dependent constraints. It also is desirable to maintain an additional separate cell culture; one which does not receive the agent being tested as a control.

As is apparent to one of skill in the art, the method can be modified for high throughput analysis and suitable cells may be cultured in microtiter plates and several agents may be assayed at the same time by noting genotypic changes, phenotypic changes and/or cell death.

When the agent is a composition other than a DNA or RNA nucleic acid molecule, the suitable conditions comprise directly added to the cell culture or added to culture medium for addition. As is apparent to those skilled in the art, an “effective” amount must be added which can be empirically determined.

The screen involves contacting the agent with a test cell characterized by differential expression of a gene of interest and then assaying the cell for the level of said gene expression. In some aspects, it may be necessary to determine the level of gene expression prior to the assay. This provides a base line to compare expression after administration of the agent to the cell culture. In another embodiment, the test cell is a cultured cell from an established cell line that differentially expresses a gene of interest. An agent is a possible therapeutic agent if gene expression is returned (reduced or increased) to a level that is present in a cell in a normal or non-pathological state, or the cell selectively dies, or exhibits reduced rate of growth.

In yet another aspect, the test cell or tissue sample is isolated from the subject to be treated and one or more potential agents are screened to determine the optimal therapeutic and/or course of treatment for that individual patient.

For the purposes of this invention, an “agent” is intended to include, but not be limited to a biological or chemical compound such as a simple or complex organic or inorganic molecule, a peptide, a protein or an oligonucleotide. A vast array of compounds can be synthesized, for example oligomers, such as oligopeptides and oligonucleotides, and synthetic organic compounds based on various core structures, and these are also included in the term “agent”. In addition, various natural sources can provide compounds for screening, such as plant or animal extracts, and the like. It should be understood, although not always explicitly stated that the agent is used alone or in combination with another agent, having the same or different biological activity as the agents identified by the inventive screen. The agents and methods also are intended to be combined with other therapies.

As noted above, cells having differential expression of a gene of interest that results in the pathological state are suitably treated by this method. These cells can be identified by any method known in the art that allows for the identification of differential expression of the gene.

When the agent is a nucleic acid, it can be added to the cell cultures by methods known in the art, which includes, but is not limited to calcium phosphate precipitation, microinjection or electroporation. Alternatively or additionally, the nucleic acid can be incorporated into an expression or insertion vector for incorporation into the cells. Vectors that contain both a promoter and a cloning site into which a polynucleotide can be operatively linked are known in the art and briefly described infra.

Polynucleotides are inserted into vector genomes using methods known in the art. For example, insert and vector DNA can be contacted, under suitable conditions, with a restriction enzyme to create complementary ends on each molecule that can pair with each other and be joined together with a ligase. Alternatively, synthetic nucleic acid linkers can be ligated to the termini of restricted polynucleotide. These synthetic linkers contain nucleic acid sequences that correspond to a particular restriction site in the vector DNA. Additionally, an oligonucleotide containing a termination codon and an appropriate restriction site can be ligated for insertion into a vector containing, for example, some or all of the following: a selectable marker gene, such as the neomycin gene for selection of stable or transient transfectants in mammalian cells; enhancer/promoter sequences from the immediate early gene of human CMV for high levels of transcription; transcription termination and RNA processing signals from SV40 for mRNA stability; SV40 polyoma origins of replication and ColE1 for proper episomal replication; versatile multiple cloning sites; and T7 and SP6 RNA promoters for in vitro transcription of sense and antisense RNA. Other means are known and available in the art.

One can determine if the object of the method, i.e., reversal of the pathological state of the cell, using methods known in the art, some of which are described herein.

When the subject is an animal such as a rat or mouse, the method provides a convenient animal model system which can be used prior to clinical testing of the therapeutic agent or alternatively, for lead optimization. In this system, a candidate agent is a potential drug if gene expression is returned to a normal level or if symptoms associated or correlated to the presence of cells containing differential expression of a gene of interest are ameliorated, each as compared to untreated, animal having the pathological cells. It also can be useful to have a separate negative control group of cells or animals which are healthy and not treated, which provides a basis for comparison. T

Therapeutic Methods

The polypeptides and polynucleotides of the invention are useful to modulate immune responses by administration to a subject, alone or in combination with vaccines, immune effector cells, antigen presenting cells, immunogens, and other known biological modifiers. For the purpose of illustration only, suitable biological modifiers include, but are not limited to, cytokines such as IL-2, IL-4, IL-10, TNF-α, IL-12, IFN-γ; non-specific modifiers such as phytohemagglutinin (PHA), phorbol esters such as phorbol myristate acetate (PMA), concanavalin-A, and ionomycin; antibodies specific for cell surface markers, such as anti-CD2, anti-CD3, anti-IL-2 receptor, anti-CD28; chemokines, including, for example, lymphotactin. The biological modifiers can be native factors obtained from natural sources, factors produced by recombinant DNA technology, chemically synthesized polypeptides or other molecules, or any derivative having the functional activity of the native factor. If more than one biological modifier is used, the exposure can be simultaneous or sequential.

In one embodiment, the composition of this invention is administered in an amount effective to modulate the immune response. In a further preferred embodiment, an agent of the invention is administered in an amount effective to treat cancer, autoimmune disease or viral infections. The compositions of the invention can also be used to prevent progression from a pre-pathological or non-malignant state into a pathological or a malignant state.

Various delivery systems are known and can be used to administer a composition or other therapeutic agent of the invention, e.g., encapsulation in liposomes, microparticles, microcapsules, expression by recombinant cells, receptor-mediated endocytosis (See, e.g., Wu and Wu, (1987), J. Biol. Chem. 262:4429-4432), construction of a therapeutic nucleic acid as part of a retroviral or other vector, etc. Methods of delivery include but are not limited to intra-arterial, intra-muscular, intravenous, intranasal, and oral routes. In a specific embodiment, it may be desirable to administer the pharmaceutical compositions of the invention locally to the area in need of treatment; this may be achieved by, for example, and not by way of limitation, local infusion during surgery, by injection, or by means of a catheter.

The compositions can be administered to subjects or individuals susceptible to or at risk of developing a disease or pathological condition. When the composition is administered to a subject such as a mouse, a rat or a human patient, the composition can be added to a pharmaceutically acceptable carrier and systemically or topically administered to the subject. To determine patients that can be beneficially treated, a tumor sample is removed from the patient and the cells are assayed for the differential expression of the gene. Therapeutic amounts can be empirically determined and will vary with the pathology being treated, the subject being treated and the efficacy and toxicity of the composition. When delivered to an animal, the method is useful to further confirm efficacy of the composition. As an example of an animal model, groups of nude mice (Balb/c NCR nu/nu female, Simonsen, Gilroy, Calif.) are each subcutaneously inoculated with about 10⁵ to about 10⁹ hyperproliferative, cancer or target cells as defined herein. When the tumor is established, the composition is administered, for example, by subcutaneous injection around the tumor. Tumor measurements to determine reduction of tumor size are made in two dimensions using venier calipers twice a week. Other animal models may also be employed as appropriate.

Administration in vivo can be effected in one dose, continuously or intermittently throughout the course of treatment. Methods of determining the most effective means and dosage of administration are known to those of skill in the art and will vary with the composition used for therapy, the purpose of the therapy, the target cell being treated, and the subject being treated. Single or multiple administrations can be carried out with the dose level and pattern being selected by the treating physician. Suitable dosage formulations and methods of administering the compositions can be found below.

The compositions of the present invention can be used in the manufacture of medicaments and for the treatment of humans and other animals by administration in accordance with conventional procedures, such as an active ingredient in pharmaceutical compositions.

The pharmaceutical compositions can be administered orally, intranasally, parenterally or by inhalation therapy, and may take the form of tablets, lozenges, granules, capsules, pills, ampoules, suppositories or aerosol form. They may also take the form of suspensions, solutions and emulsions of the active ingredient in aqueous or nonaqueous diluents, syrups, granulates or powders. In addition to an agent of the present invention, the pharmaceutical compositions can also contain other pharmaceutically active compounds or a plurality of compounds of the invention.

More particularly, an agent of the present invention also referred to herein as the active ingredient, may be administered for therapy by any suitable route including oral, rectal, nasal, topical (including transdermal, aerosol, buccal and sublingual), vaginal, parental (including subcutaneous, intramuscular, intravenous and intradermal) and pulmonary. It will also be appreciated that the preferred route will vary with the condition and age of the recipient, and the disease being treated.

Ideally, the composition should be administered to achieve peak concentrations of the active compound at sites of disease. This may be achieved, for example, by the intravenous injection of the composition, optionally in saline, or orally administered, for example, as a tablet, capsule or syrup containing the active ingredient. Desirable blood levels of the composition may be maintained by a continuous infusion to provide a therapeutic amount of the active ingredient within disease tissue. The use of operative combinations is contemplated to provide therapeutic combinations requiring a lower total dosage of each component than may be required when each individual therapeutic compound or drug is used alone, thereby reducing adverse effects.

While it is possible for the composition to be administered alone, it is preferable to present it as a pharmaceutical formulation comprising at least one active ingredient, as defined above, together with one or more pharmaceutically acceptable carriers therefor and optionally other therapeutic compositions. Each carrier must be “acceptable” in the sense of being compatible with the other ingredients of the formulation and not injurious to the patient.

Formulations include those suitable for oral, rectal, nasal, topical (including transdermal, buccal and sublingual), vaginal, parenteral (including subcutaneous, intramuscular, intravenous and intradermal) and pulmonary administration. The formulations may conveniently be presented in unit dosage form and may be prepared by any methods known in the art of pharmacy. Such methods include the step of bringing into association the active ingredient with the carrier which constitutes one or more accessory ingredients. In general, the formulations are prepared by uniformly and intimately bringing into association the active ingredient with liquid carriers or finely divided solid carriers or both, and then if necessary shaping the product.

Formulations of the present invention suitable for oral administration may be presented as discrete units such as capsules, cachets or tablets, each containing a predetermined amount of the active ingredient; as a powder or granules; as a solution or suspension in an aqueous or non-aqueous liquid; or as an oil-in-water liquid emulsion or a water-in-oil liquid emulsion. The active ingredient may also be presented a bolus, electuary or paste.

A tablet may be made by compression or molding, optionally with one or more accessory ingredients. Compressed tablets may be prepared by compressing in a suitable machine the active ingredient in a free-flowing form such as a powder or granules, optionally mixed with a binder (e.g., povidone, gelatin, hydroxypropylmethyl cellulose), lubricant, inert diluent, preservative, disintegrant (e.g., sodium starch glycolate, cross-linked povidone, cross-linked sodium carboxymethyl cellulose) surface-active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein using, for example, hydroxypropylmethyl cellulose in varying proportions to provide the desired release profile. Tablets may optionally be provided with an enteric coating, to provide release in parts of the gut other than the stomach.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Pharmaceutical compositions for topical administration according to the present invention may be formulated as an ointment, cream, suspension, lotion, powder, solution, past, gel, spray, aerosol or oil. Alternatively, a formulation may comprise a patch or a dressing such as a bandage or adhesive plaster impregnated with active ingredients and optionally one or more excipients or diluents.

If desired, the aqueous phase of the cream base may include, for example, at least about 30% w/w of a polyhydric alcohol, i.e., an alcohol having two or more hydroxyl groups such as propylene glycol, butane-1,3-diol, mannitol, sorbitol, glycerol and polyethylene glycol and mixtures thereof. The topical formulations may desirably include a compound which enhances absorption or penetration of the composition through the skin or other affected areas. Examples of such dermal penetration enhancers include dimethylsulfoxide and related analogues.

The oily phase of the emulsions of this invention may be constituted from known ingredients in a known manner. While this phase may comprise merely an emulsifier (otherwise known as an emulgent), it desirably comprises a mixture of at lease one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make up the so-called emulsifying wax, and the wax together with the oil and/or fat make up the so-called emulsifying ointment base which forms the oily dispersed phase of the cream formulations.

Emulgents and emulsion stabilizers suitable for use in the formulation of the present invention include Tween 60, Span 80, cetostearyl alcohol, myristyl alcohol, glyceryl monostearate and sodium lauryl sulphate.

The choice of suitable oils or fats for the formulation is based on achieving the desired cosmetic properties, since the solubility of the active compound in most oils likely to be used in pharmaceutical emulsion formulations is very low. Thus the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for topical administration to the eye also include eye drops wherein the active ingredient is dissolved or suspended in a suitable carrier, especially an aqueous solvent for the composition.

Formulations for rectal administration may be presented as a suppository with a suitable base comprising, for example, cocoa butter or a salicylate.

Formulations suitable for vaginal administration may be presented as pessaries, tampons, creams, gels, pastes, foams or spray formulations containing in addition to the composition, such carriers as are known in the art to be appropriate.

Formulations suitable for nasal administration, wherein the carrier is a solid, include a coarse powder having a particle size, for example, in the range of about 20 to about 500 microns which is administered in the manner in which snuff is taken, i.e., by rapid inhalation through the nasal passage from a container of the powder held close up to the nose. Suitable formulations wherein the carrier is a liquid for administration as, for example, nasal spray, nasal drops, or by aerosol administration by nebulizer, include aqueous or oily solutions of the composition.

Formulations suitable for parenteral administration include aqueous and non-aqueous isotonic sterile injection solutions which may contain anti-oxidants, buffers, bacteriostats and solutes which render the formulation isotonic with the blood of the intended recipient; and aqueous and non-aqueous sterile suspensions which may include suspending agents and thickening agents, and liposomes or other microparticulate systems which are designed to target the compound to blood components or one or more organs. The formulations may be presented in unit-dose or multi-dose sealed containers, for example, ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets of the kind previously described.

Preferred unit dosage formulations are those containing a daily dose or unit, daily subdose, as herein above-recited, or an appropriate fraction thereof, of an composition.

It should be understood that in addition to the ingredients particularly mentioned above, the formulations of this invention may include other agents conventional in the art having regard to the type of formulation in question, for example, those suitable for oral administration may include such further agents as sweeteners, thickeners and flavoring agents. It also is intended that the agents, compositions and methods of this invention be combined with other suitable compositions and therapies.

Transgenic Animals

In another aspect, the genes of Table 1 can be used to generate transgenic animal models. In recent years, geneticists have succeeded in creating transgenic animals, for example mice, by manipulating the genes of developing embryos and introducing foreign genes into these embryos. Once these genes have integrated into the genome of the recipient embryo, the resulting embryos or adult animals can be analyzed to determine the function of the gene. The mutant animals are produced to understand the function of known genes in vivo and to create animal models of human diseases. (See, e.g., Chisaka et al. (1992) 355:516-520; Joyner et al. (1992) in POSTIMPLANTATION DEVELOPMENT IN THE MOUSE (Chadwick and Marsh, eds., John Wiley & Sons, United Kingdom) pp:277-297; Dorin et al. (1992) Nature 359:211-215).

U.S. Pat. Nos. 5,464,764 and 5,487,992 describe one type of transgenic animal in which the gene of interest is deleted or mutated sufficiently to disrupt its function. (See, also U.S. Pat. Nos. 5,631,153 and 5,627,059). These “knock-out” animals, made by taking advantage of the phenomena of homologous recombination, can be used to study the function of a particular gene sequence in vivo.

Antibodies

Also provided by this invention is an antibody capable of specifically forming a complex with the expression product of a gene of interest. The term “antibody” includes polyclonal antibodies and monoclonal antibodies. The antibodies include, but are not limited to mouse, rat, and rabbit or human antibodies. The antibodies are useful to identify and purify gene expression products as well as APCs expressing the polypeptides.

Laboratory methods for producing polyclonal antibodies and monoclonal antibodies, as well as deducing their corresponding nucleic acid sequences, are known in the art, see Harlow and Lane (1988) supra and Sambrook et al. (1989) supra. The monoclonal antibodies of this invention can be biologically produced by introducing protein or a fragment thereof into an animal, e.g., a mouse or a rabbit. The antibody producing cells in the animal are isolated and fused with myeloma cells or heteromyeloma cells to produce hybrid cells or hybridomas. Accordingly, the hybridoma cells producing the monoclonal antibodies of this invention also are provided.

Thus, using the protein or fragment thereof, and known methods, one of skill in the art can produce and screen the hybridoma cells and antibodies of this invention for antibodies having the ability to bind the proteins or polypeptides.

If a monoclonal antibody being tested binds with the protein or polypeptide, then the antibody being tested and the antibodies provided by the hybridomas of this invention are equivalent. It also is possible to determine without undue experimentation, whether an antibody has the same specificity as the monoclonal antibody of this invention by determining whether the antibody being tested prevents a monoclonal antibody of this invention from binding the protein or polypeptide with which the monoclonal antibody is normally reactive. If the antibody being tested competes with the monoclonal antibody of the invention as shown by a decrease in binding by the monoclonal antibody of this invention, then it is likely that the two antibodies bind to the same or a closely related epitope. Alternatively, one can pre-incubate the monoclonal antibody of this invention with a protein with which it is normally reactive, and determine if the monoclonal antibody being tested is inhibited in its ability to bind the antigen. If the monoclonal antibody being tested is inhibited then, in all likelihood, it has the same, or a closely related, epitopic specificity as the monoclonal antibody of this invention.

The term “antibody” also is intended to include antibodies of all isotypes. Particular isotypes of a monoclonal antibody can be prepared either directly by selecting from the initial fusion, or prepared secondarily, from a parental hybridoma secreting a monoclonal antibody of different isotype by using the sib selection technique to isolate class switch variants using the procedure described in Steplewski et al. (1985) Proc. Natl. Acad. Sci. USA 82:8653 or Spira et al. (1984) J. Immunol. Meth. 74:307.

This invention also provides biological active fragments of the polyclonal and monoclonal antibodies described above. These “antibody fragments” retain some ability to selectively bind with its antigen or immunogen. Such antibody fragments can include, but are not limited to

Fab

Fab′

F(ab′)₂,

Fv, and

SCA

A specific example of “a biologically active antibody fragment” is a CDR region of the antibody. Methods of making these fragments are known in the art, see for example, Harlow and Lane (1988) supra.

The antibodies of this invention also can be modified to create chimeric antibodies and humanized antibodies (Oi et al. (1986) BioTechniques 4(3):214). Chimeric antibodies are those in which the various domains of the antibodies' heavy and light chains are coded for by DNA from more than one species.

The isolation of other hybridomas secreting monoclonal antibodies with the specificity of the monoclonal antibodies of the invention can also be accomplished by one of ordinary skill in the art by producing anti-idiotypic antibodies (Herlyn et al. (1986) Science 232:100). An anti-idiotypic antibody is an antibody which recognizes unique determinants present on the monoclonal antibody produced by the hybridoma of interest.

Idiotypic identity between monoclonal antibodies of two hybridomas demonstrates that the two monoclonal antibodies are the same with respect to their recognition of the same epitopic determinant. Thus, by using antibodies to the epitopic determinants on a monoclonal antibody it is possible to identify other hybridomas expressing monoclonal antibodies of the same epitopic specificity.

It is also possible to use the anti-idiotype technology to produce monoclonal antibodies which mimic an epitope. For example, an anti-idiotypic monoclonal antibody made to a first monoclonal antibody will have a binding domain in the hypervariable region which is the mirror image of the epitope bound by the first monoclonal antibody. Thus, in this instance, the anti-idiotypic monoclonal antibody could be used for immunization for production of these antibodies.

As used in this invention, the term “epitope” is meant to include any determinant having specific affinity for the monoclonal antibodies of the invention. Epitopic determinants usually consist of chemically active surface groupings of molecules such as amino acids or sugar side chains and usually have specific three dimensional structural characteristics, as well as specific charge characteristics.

The antibodies of this invention can be linked to a detectable agent or label. There are many different labels and methods of labeling known to those of ordinary skill in the art.

The coupling of antibodies to low molecular weight haptens can increase the sensitivity of the assay. The haptens can then be specifically detected by means of a second reaction. For example, it is common to use haptens such as biotin, which reacts avidin, or dinitrophenol, pyridoxal, and fluorescein, which can react with specific anti-hapten antibodies. See, Harlow and Lane (1988) supra.

The monoclonal antibodies of the invention also can be bound to many different carriers. Thus, this invention also provides compositions containing the antibodies and another substance, active or inert. Examples of well-known carriers include glass, polystyrene, polypropylene, polyethylene, dextran, nylon, amylases, natural and modified celluloses, polyacrylamides, agaroses and magnetite. The nature of the carrier can be either soluble or insoluble for purposes of the invention. Those skilled in the art will know of other suitable carriers for binding monoclonal antibodies, or will be able to ascertain such, using routine experimentation.

Compositions containing the antibodies, fragments thereof or cell lines which produce the antibodies, are encompassed by this invention. When these compositions are to be used pharmaceutically, they are combined with a pharmaceutically acceptable carrier.

Specific combination(s) of cytokines have been used successfully to amplify (or partially substitute) for the activation/conversion achieved with calcium ionophore: these cytokines include but are not limited to purified or recombinant human (“rh”) rhGM-CSF, rhIL-2, and rhIL-4. Each cytokine when given alone is inadequate for optimal upregulation.

Materials and Methods

Phenotypic Characterization of Invention Polypeptides

These methods are provided herein to determine the optimal invention polypeptide for treatment of a disease or for personalized therapy.

A polyncucleotide, e.g., cDNA sequence, encoding an invention polypeptide is cloned into a plasmid expression vector, and placed under the control of a strong promoter such as CMV. The cDNA sequences are inserted in frame and cloned 5′ of a short sequence encoding a convenient epitope tag. The encoded secreted factor possesses the epitope tag appended to its carboxy terminus. Plasmids encoding tagged candidate factors are transfected into cell lines such as HEK293 or COS cells. Conditioned medium derived from transfected cells is harvested and epitope tagged proteins are immunoprecipitated by using anti-epitope tag antibodies. Immunoprecipitated proteins are detected following polyacrylamide gel electrophoresis and the molecular mass of the candidate factor derived from the conditioned medium is determine and compared to the mass predicted on the basis of DNA sequence data.

The in vitro biological activity of the secreted polypeptide is then confirmed. Transfected cells producing secreted factors detected above are separated from target cells by a permeable membrane in a transwell tissue culture plate and the effect of the secreted factor on the behavior of target cells is determined. Chemotactic factors such as chemokines will stimulate the migration of the target cells. Alternatively, conditioned media from plasmid-transfected cells can be added directly to target cells. Growth factors will stimulate the proliferation of target cells. Cytokines would be predicted to stimulate the proliferation or activation of immune effector cells.

In Vitro Assays

Migration Assay

Cell lines transfected with plasmids encoding candidate secreted factors are placed into the lower chamber of a transwell chamber. See, e.g., Fan & Malik (2003) Nature Medicine 9:315. Target cells, potentially responsive to the effects of the secreted factor, are added to the upper chamber of the transwell plate where they can be separated from the transfected cells by a permeable membrane. The target cells can be labeled (chemical, fluorescent, radioactive) to facilitate quantitation and detection. Suitable target cells include, but are not limited to monocytes, T cells, endothelial cells, dendritic cells, macrophages or other cell types potentially capable of responding to external soluble factors. The labeled cells are monitored as a function of time to determine the proportion that migrate through the permeable membrane and enter the lower chamber in response to chemotactic proteinaceous factors produced by the transfected cells. In instances where the concentration of the secreted factor within the conditioned medium is not high enough to detect a chemotactic activity, conditioned media can be collected, and concentrated prior to addition to the lower chamber using the method described in Cao et al. (2000) J. Immunology 1652588. To confirm the observed effect is due to the secreted factor, anti-epitope tag antibodies can be used to deplete conditioned medium of the secreted factor. Vigne et al. (2001) J. Biol. Chem. 276:17101.

To confirm that the agent is to alter or modulate soluble factors capable of inducing transendothelial migration of T cells of other cells, the insert of a transwell plate can be coated with endothelial cells to produce a confluent monolayer. The cell-coated permeable insert would be placed between target cells (T cells or other) in the upper chamber and gene transfected cells or conditioned medium from gene transfected cells in the lower chamber. As above, the number of target cells that are above to traverse the endothelial cell-coated membrane is determined.

Proliferation

Target cells, potentially responsive to the effects of the secreted factor, are added to a well of a tissue culture plate. Conditioned medium from gene transfected cells are added to the well along with ³H-thymidine. Target cells could include, but are not limited to monocytes, T cells, endothelial cells, dendritic cells, macrophages or other cell types potentially capable of responding to external soluble factors. Following several days, the level of ³H-thymidine incorporated into target cells is determined by scintillation counting. To confirm the observed effect is due to the candidate secreted factor, anti-epitope tag antibodies can be used to deplete conditioned medium of the secreted factor. The assay can also be conducted using transwell plates by adding target cells to the upper chamber and plasmid-transfected, irradiated cells to the lower chamber. Incorporation of ³H-thymidine into live cells in the upper chamber can be determined while no radioactivity would be incorporated into irradiated cells in the lower chamber.

Activation

Target cells, potentially responsive to the effects of the secreted factor, are added to a well of a micro culture plate. Conditioned medium from gene transfected cells is then added to the well. Target cells include, but are not limited to monocytes, T cells, endothelial cells, dendritic cells, macrophages or other cell types potentially capable of responding to external soluble factors. Following several hours, labeled antibodies specific for activation markers displayed on the target cells or produced by the target cells, are added to the well and the intensity of the antibody signal could be quantitated. Soluble factors such as cytokines, capable of inducing the upregulation of activation markers, can be detected. To confirm the observed effect is due to the secreted factor, anti-epitope tag antibodies can be used to deplete conditioned medium of the secreted factor. The assay can also be conducted using transwell plates by adding target cells to the upper chamber and plasmid transfected cells to the lower chamber.

In Vivo Assays

High pressure tail vein plasmid injection could be conducted to force expression of the soluble factor in the liver of recipient rodents. Anti-epitope tag antibodies can be used to detect the presence of the secreted tagged protein in the blood of injected animals by immunoprecipitation followed by gel electrophoresis. At various time points following plasmid injection, blood samples are drawn and cellular and chemical analyses is conducted. Growth factors might be expected to cause the proliferation of specific circulating blood cell types that could be enumerated by conducting FACS using cell type specific antibodies. Activating factors might be expected to promote the display of activation markers on specific cell types that could be detected by FACS using marker specific antibodies. Finally liver biopsies can be collected from from plasmid injected animals and cellular infiltrates are detected and identified by conducting histology and immunohistochemistry. Chemotactic factors are expected to cause the recruitment and concentration of specific circulating cell types within the liver. Alternatively, subcutaneous or intradermal injection of plasmids encoding candidate chemotactic factors or of cells producing candidate soluble factors can be performed. Fan & Malik (2003) Nature Medicine 9:315. Skin biopsies or injection site lavages can be harvested from injected animals and cellular infiltrates could be detected and identified by conducting histology and immunohistochemistry.

Therapeutic Methods

Enhancement of Antigens by Antigen-Presenting Matrices

The compositions of this invention can be used in conjunction with antigen-presenting matrices. For use in immunomodulatory methods and diagnostic methods of the invention, an antigen-presenting matrix can presents an antigenic epitope bound to an MHC molecule. Any known method can be used to achieve presentation of an epitope by an antigen-presenting matrix. The following are non-limiting examples of methods which can be used.

An antigenic peptide can be delivered to antigen-presenting cells as polypeptide or peptide or in the form of cDNA encoding the protein/peptide.

Another method to deliver an antigenic epitope to an APC is by pulsing. Pulsing can be accomplished in vitro/ex vivo by exposing APCs to the antigenic polypeptide(s) or peptide(s) of this invention. The polypeptide(s) or peptide(s) are added to APCs at a concentration of 1-10 μm for approximately 3 hours. Pulsed APCs can subsequently be administered to the host via an intravenous, subcutaneous, intranasal, intramuscular or intraperitoneal route of delivery.

Antigenic epitopes can also be delivered in vivo, for example, as part of a polypeptide or complexed with another macromolecule, with or without adjuvant via the intravenous, subcutaneous, intranasal, intramuscular or intraperitoneal route of delivery.

Various other techniques can be used, including the following. Paglia et al. (1996) J. Exp. Med. 183:317-322 has shown that APC incubated with whole protein in vitro are recognized by MHC Class I-restricted CTLs, and that immunization of animals with these APCs led to the development of antigen-specific CTLs in vivo. In addition, several different techniques have been described which lead to the expression of antigen in the cytosol of APCs, such as DCs. These include (1) the introduction into the APCs of RNA isolated from tumor cells, (2) infection of APCs with recombinant vectors to induce endogenous expression of antigen, and (3) introduction of tumor antigen into the DC cytosol using liposomes. See Boczkowski et al. (1996) J. Exp. Med. 184:465-472; Rouse et al. (1994) J. Virol. 68:5685-5689; and Nair et al. (1992) J. Exp. Med. 175:609-612).

Another method which can be used is termed “painting.” It has been demonstrated that glycosyl-phosphotidylinositol (GPI)-modified proteins possess the ability to reincorporate themselves back into cell membranes after purification. Hirose et al. (1995) Methods Enzymol. 250:582-614; Medof et al., (1984) J. Exp. Med. 160:1558-1578; Medof (1996) FASEB J. 10:574-586; and Huang et al. (1994) Immunity 1:607-613 have exploited this property in order to create APCs of specific composition for the presentation of antigen to CTLs. They devised expression vectors for β2-microglobulin and the HLA-A2.1 allele. The proteins were expressed in Schneider S2 Drosophila melanogaster cells, known to support GPI-modification. After purification, the proteins could be incubated together with a purified antigenic peptide which resulted in a trimolecular complex capable of efficiently inserting itself into the membranes of autologous cells. In essence, these protein mixtures were used to “paint” the APC surface, conferring the ability to stimulate a CTL clone that was specific for the antigenic peptide. Cell coating was shown to occur rapidly and to be protein concentration dependent. This method of generating APCs bypasses the need for gene transfer into the APC and permits control of antigenic peptide densities at the cell surfaces.

Stimulation of Immune Effector Cells

The present invention makes use of the above-described antigen-presenting matrices, including APCs, to stimulate production of an enriched population of antigen-specific immune effector cells. The APCs prepared as described above are mixed with naïve immune effector cells. Preferably, the cells are cultured in the presence of a suitable polypeptide of this invention. The culture conditions are such that the antigen-specific immune effector cells expand (i.e. proliferate) at a much higher rate than the APCs. Multiple infusions of APCs and optional cytokines can be performed to further expand the population of antigen-specific cells.

In one embodiment, the immune effector cells are T cells. In a separate embodiment, the immune effector cells can be genetically modified by transduction with a transgene coding for a polypeptide of this invention. Methods for introducing transgenes in vitro, ex vivo and in vivo are known in the art. See Sambrook, et al. (1989) supra.

An effector cell population suitable for use in the methods of the present invention can be autogeneic or allogeneic, preferably autogeneic. When effector cells are allogeneic, preferably the cells are depleted of alloreactive cells before use. This can be accomplished by any known means, including, for example, by mixing the allogeneic effector cells and a recipient cell population and incubating them for a suitable time, then depleting CD69⁺ cells, or inactivating alloreactive cells, or inducing anergy in the alloreactive cell population.

Hybrid immune effector cells can also be used. Immune effector cell hybrids are known in the art and have been described in various publications. See, for example, International Patent Application Nos. WO 98/46785; and WO 95/16775.

The effector cell population can comprise unseparated cells, i.e., a mixed population, for example, a PBMC population, whole blood, and the like. The effector cell population can be manipulated by positive selection based on expression of cell surface markers, negative selection based on expression of cell surface markers, stimulation with one or more antigens in vitro or in vivo, treatment with one or more polynucleotides and/or polypeptides of the invention, alone or in combination with biological modifiers in vitro or in vivo, subtractive stimulation with one or more antigens or biological modifiers, or a combination of any or all of these.

Effector cells can obtained from a variety of sources, including but not limited to, PBMC, whole blood or fractions thereof containing mixed populations, spleen cells, bone marrow cells, tumor infiltrating lymphocytes, cells obtained by leukapheresis, biopsy tissue, lymph nodes, e.g., lymph nodes draining from a tumor. Suitable donors include an immunized donor, a non-immunized (naïve) donor, treated or untreated donors. A “treated” donor is one that has been exposed to one or more biological modifiers. An “untreated” donor has not been exposed to one or more biological modifiers.

Methods of extracting and culturing effector cells are known in the art. For example, effector cells can be obtained by leukapheresis, mechanical apheresis using a continuous flow cell separator. For example, lymphocytes and monocytes can be isolated from the buffy coat by any known method, including, but not limited to, separation over Ficoll-Hypaque™ gradient, separation over a Percoll gradient, or elutriation. The concentration of Ficoll-Hypaque™ can be adjusted to obtain the desired population, for example, a population enriched in T cells. Other methods based on affinity are known and can be used. These include, for example, fluorescence-activated cell sorting (FACS), cell adhesion, magnetic bead separation, and the like. Affinity-based methods may utilize antibodies, or portions thereof, which are specific for cell-surface markers and which are available from a variety of commercial sources, including, the American Type Culture Collection (Manassas, Md.). Affinity-based methods can alternatively utilize ligands or ligand analogs, of cell surface receptors.

The effector cell population can be subjected to one or more separation protocols based on the expression of cell surface markers. For example, the cells can be subjected to positive selection on the basis of expression of one or more cell surface polypeptides, including, but not limited to, “cluster of differentiation” cell surface markers such as CD2, CD3, CD4, CD8, TCR, CD45, CD45RO, CD45RA, CD11b, CD26, CD27, CD28, CD29, CD30, CD31, CD40L; other markers associated with lymphocyte activation, such as the lymphocyte activation gene 3 product (LAG3), signaling lymphocyte activation molecule (SLAM), TI/ST2; chemokine receptors such as CCR3, CCR4, CXCR3, CCR5; homing receptors such as CD62L, CD44, CLA, CD146, a4b7, aEb7; activation markers such as CD25, CD69 and OX40; and lipoglycans presented by CD1. The effector cell population can be subjected to negative selection for depletion of non-T cells and/or particular T cell subsets. Negative selection can be performed on the basis of cell surface expression of a variety of molecules, including, but not limited to, B cell markers such as CD19, and CD20; monocyte marker CD14; the NK cell marker CD56.

Various methods are known to evaluate T cell activation. CTL activation can be detected by any known method, including but not limited to, tritiated thymidine incorporation (indicative of DNA synthesis), and examination of the population for growth or proliferation, e.g., by identification of colonies. Alternatively, the tetrazolium salt MTT (3-(4,5-dimethyl-thazol-2-yl)-2,5-diphenyl tetrazolium bromide) may be added. Mossman (1983) J. Immunol. Methods 65:55-63; Niks and Otto (1990) J. Immunol. Methods 130:140-151. Succinate dehydrogenase, found in mitochondria of viable cells, converts the MTT to formazan blue. Thus, concentrated blue color would indicate metabolically active cells. In yet another embodiment, incorporation of radiolabel, e.g., tritiated thymidine, may be assayed to indicate proliferation of cells. Similarly, protein synthesis may be shown by incorporation of ³⁵S-methionine. In still another embodiment, cytotoxicity and cell killing assays, such as the classical chromium release assay, may be employed to evaluate epitope-specific CTL activation. To detect activation of CD4+ T cells, any of a variety of methods can be used, including, but not limited to, measuring cytokine production; and proliferation, for example, by tritiated thymidine incorporation

Release of ⁵¹Cr from labeled target cells is a standard assay which can be used to assess the number of peptide-specific CTLs in a biological sample. Tumor cells, or APCs of the invention, are radiolabeled as targets with about 200 μCi of Na₂ ⁵¹CrO₄ for 60 minutes at 37° C., followed by washing. T cells and target cells (˜1×10⁴/well) are then combined at various effector-to-target ratios in 96-well, U-bottom plates. The plates are centrifuged at 100×g for 5 minutes to initiate cell contact, and are incubated for 4-16 hours at 37° C. with 5% CO₂. Release of ⁵¹Cr is determined in the supernatant, and compared with targets incubated in the absence of T cells (negative control) or with 0.1% TRITON™ X-100 (positive control). See, e.g., Mishell and Shiigi, eds. SELECTED METHODS IN CELLULAR IMMUNOLOGY (1980) W.H. Freeman and Co.

Polynucleotides of the invention can be administered in a gene delivery vehicle or by inserting into a host cell which in turn recombinantly transcribes, translates and processed the encoded polypeptide. Isolated host cells containing a polynucleotide of the invention in a pharmaceutically acceptable carrier can be combined with appropriate and effective amount of an adjuvant or other biological modifier. In some embodiments, the host cell is an APC, such as a dendritic cell. The agents provided herein as effective for their intended purpose can be administered to subjects having a disease to be treated with an immunomodulatory method of the invention or to individuals susceptible to or at risk of developing such a disease. When the agent is administered to a subject such as a mouse, a rat or a human patient, the agent can be added to a pharmaceutically acceptable carrier and systemically or topically administered to the subject. Therapeutic amounts can be empirically determined and will vary with the pathology or condition being treated, the subject being treated and the efficacy and toxicity of the therapy.

The amount of a peptide or immune effector cell of the invention will vary depending, in part, on its intended effect, and is ultimately at the discretion of the medical or veterinary practitioner. The factors to be considered include the condition being treated, the route of administration, and nature of the formulation, the mammal's body weight, surface area, age, and general condition and the particular peptide to be administered. A suitable effective dose of peptides of the invention generally lies in the range of from about 0.0001 μmol/kg to about 1000 μmol/kg bodyweight. The total dose may be given as a single dose or multiple doses, e.g., two to six times per day. For example, for a 75 kg mammal (e.g., a human) the dose range would be about 2.25 μmol/kg/day and a typical dose could be about 100 μmol of peptide. If discrete multiple doses are indicated treatment might typically be 25 μmol of a peptide of the invention given up to 4 times per day. In an alternative administrative regimen, peptides of the invention may be given on alternate days or even once or twice a week. A suitable effective dose of an immune effector cell of the invention generally lies in the range of from about 10² to about 10⁹ cells per administration. Cells can be administered once, followed by monitoring of the clinical response, such as diminution of disease symptoms or tumor mass. Administration may be repeated on a monthly basis, for example, or as appropriate. Those skilled in the art will appreciate that an appropriate administrative regimen would be at the discretion of the physician or veterinary practitioner.

Administration in vivo can be effected in one dose, continuously or intermittently throughout the course of treatment. Methods of determining the most effective means and dosage of administration are known to those of skill in the art and will vary with the composition used for therapy, the purpose of the therapy, the target cell being treated, and the subject being treated. Single or multiple administrations can be carried out with the dose level and pattern being selected by the treating physician. Suitable dosage formulations and methods of administering the agents can be found below.

The agents and compositions of the present invention can be used in the manufacture of medicaments and for the treatment of humans and other animals by administration in accordance with conventional procedures, such as an active ingredient in pharmaceutical compositions.

Enhancement of Vaccines for Cancer Treatment and Prevention

The compositions of this invention can be used in conjunction with vaccines for cancer treatment. Cancer cells contain many new antigens potentially recognizable by the immune system. Given the speed with which epitopes can be identified, custom anticancer vaccines can be generated for affected individuals by isolating TILs from patients with solid tumors, determining their MHC restriction, and assaying these CTLs against the appropriate library for reactive epitopes. These vaccines will be both treatments for affected individuals as well as preventive therapy against recurrence (or establishment of the disease in patients which present with a familial genetic predisposition to it). Inoculation of individuals who have never had the cancer is expected to be quite successful as preventive therapy, even though a tumor antigen-specific CTL response has not yet been elicited, because in most cases high affinity peptides seem to be immunogenic suggesting that holes in the functional T cell repertoire, if they exist, may be relatively rare. Sette et al. (1994) J. Immunol., 153:5586-5592. In mice, vaccination with appropriate epitopes not only eliminates established tumors but also protects against tumor re-establishment after inoculation with otherwise lethal doses of tumor cells. Bystryn et al. (1993) supra.

Recent advances in vaccine adjuvants provide effective means of administering peptides so that they impact maximally on the immune system. Del-Giudice (1994) Experientia 50:1061-1066. These peptide vaccines will be of great value in treating metastatic tumors that are generally unresponsive to conventional therapies. Tumors arising from the homozygous deletion of recessive oncogenes are less susceptible to elimination by a humoral (antibody) response and would thus be treated more effectively by eliciting a cellular, CTL response.

Enhancement of Vaccines for Diseases Caused by Pathogenic Organisms

The compositions of this invention are also useful in methods to induce (or increase, or enhance) an immune response to a pathogenic organism. These include pathogenic viruses, bacteria, and protozoans.

Viral infections are ideal candidates for immunotherapy. Immunological responses to viral pathogens are sometimes ineffective as in the case of the lentiviruses such as HIV which causes AIDS. The high rates of spontaneous mutation make these viruses elusive to the immune system. However, a saturating profile of CTL epitopes presented on infected cells will identify shared antigens among different serotypes in essential genes that are largely intolerant to mutation which would allow the design of more effective vaccines.

Adoptive Immunotherapy Methods

The expanded populations of antigen-specific immune effector cells and APCs of the present invention find use in adoptive immunotherapy regimes and as vaccines.

Adoptive immunotherapy methods involve, in one aspect, administering to a subject a substantially pure population of educated, antigen-specific immune effector cells made by culturing naïve immune effector cells with APCs as described above. In some embodiments, the APCs are dendritic cells.

In one embodiment, the adoptive immunotherapy methods described herein are autologous. In this case, the APCs are made using parental cells isolated from a single subject. The expanded population also employs T cells isolated from that subject. Finally, the expanded population of antigen-specific cells is administered to the same patient.

In a further embodiment, APCs or immune effector cells are administered with an effective amount of a stimulatory cytokine, such as IL-2 or a co-stimulatory molecule.

Methods of Inducing T Cell Anergy

Antigenic peptides are useful in methods to induce T cell unresponsiveness, or anergy. Disorders which can be treated using these methods include autoimmune disorders, allergies, and allograft rejection.

Autoimmune disorders are diseases in which the body's immune system responds against self tissues. They include most forms of arthritis, ulcerative colitis, and multiple sclerosis. Antigenic peptides corresponding to endogenous elements that are recognized as foreign can be used in the development of treatments using gene therapy or other approaches. For example, CTL epitopes, which can act as “suicide substrates” for CTLs that mediate autoimmunity, can be designed as described above. That is to say, peptides which have a high affinity for the MHC allele but fail to activate the TCR could effectively mask the cellular immune response against cells presenting the antigen in question. In support of this approach, it is believed that the long latency period of the HIV virus is due to an antiviral immune response and a mechanism by which the virus finally evades the immune system is by generating epitopes that occupy the MHC molecules but do not stimulate a TCR lytic response, inducing specific T cell anergy. Klenerman et al. (1995) Eur. J. Immunol. 25:1927-1931.

In vitro stimulation of T cells through the complex of T cell-antigen receptor and CD3 alone in the absence of other signals, induces T cell anergy or paralysis. T cell activation as measured by interleukin-2 production and proliferation in vitro requires both antigenic and co-stimulatory signals engendered by cell to cell interactions among antigen-specific T cells and antigen presenting cells. Various interactions of these CD2 proteins on the T-cell surface with CD58 (LFA-3) proteins and antigen-presenting cells, those of CD11a/CD18 (LFA-1) proteins with CD54 (ICAM-1) proteins and those of CD5 proteins with CD72 proteins can impart such a co-stimulatory signal in vitro. Cytokines derived from antigen-presenting cells (e.g., interleukin-1 and interleukin-6) can also provide co-stimulatory signals that result in T-cell activation in vitro. The delivery of both antigenic and co-stimulatory signals leads to stable transcription of the interleukin-2 gene and other pivotal T cell-activation genes. The foregoing co-stimulatory signals depend on protein kinase C and calcium. Potent antigen presenting cells express CD80 (B7 and BB1) and other related surface proteins and many T cells express B7 binding proteins, namely CD28 and CTLA-4 proteins. Binding of CD80 by CD28 and CDLA-4 stimulates a T cell co-stimulatory pathway that is independent of protein kinase C and calcium leading to vigorous T cell proliferation. The stimulation of B cells also depends on the interaction between the specific antigen and the cell-surface immunoglobulin. T cell derived cytokines (e.g., interleukins-1 and -4), physical contact between T cells and B cells through specific pairs of receptors and co-receptors, or both, provide the signal or signals essential for B cell stimulation.

Conventional routes of administration are used. A T-cell stimulating or anergy producing amount (or therapeutically effective amount as described above) of an immunotherapeutic antigen-superantigen polymer according to the invention is contacted with the target cells. By “T-cell anergy effective amount” is intended an amount which is effective in producing a statistically significant inhibition of a cellular activity mediated by a TCR. This may be assessed in vitro using T-cell activation tests. Typically, T-cell anergy or activation is assayed by tritiated thymidine incorporation in response to specific antigen.

One way in which T cell anergy can be induced is to present to a T cell an antigen-presenting matrix which presents an antigenic peptide of the invention in an MHC Class I or Class II molecule, but which lack co-stimulatory molecules necessary to activate the T cell. For example, a cell other than a normal antigen presenting cell (APC), which has been transfected with MHC antigen to which a selected T cell clone is restricted, can be used. Resting T cells are provided with an appropriate peptide recognized by the resting T cells in the context of the MHC transfected into a cellular host other than an APC. The MHC is expressed as a result of introduction into a mammalian cell other than an antigen presenting cell of genes constitutively expressing the α and β chains of the MHC Class II, or an MHC Class I molecule together with invariant chain. Importantly, these cells do not provide other proteins, either cell surface proteins or secreted proteins, associated with antigen presenting cells, which together with the MHC and peptide result in co-stimulatory signals.

To determine whether anergy has been induced, the T cells to be tested can be cultured together with an antigen presenting matrix which presents an antigenic peptide in an MHC Class I or Class II molecule together with co-stimulatory molecules necessary to activate the T cell. The cultures are incubated for about 48 hours, then pulsed with tritiated thymidine and incorporation measured about 18 hours later. The absence of incorporation above control levels, where the T-cells are presented with antigen presenting cells which do not stimulate the T cells, either due to using an MHC to which the T cells are not restricted or using a peptide to which the T cells are not sensitive, is indicative of an absence of activation. One may use other conventional assays to determine the extent of activation, such as assaying for IL-2, -3, or -4, cell surface proteins associated with activation, e.g., CD71 or other convenient techniques. Another method is to determine the expression of a protein which is expressed on quiescent T cells, but not on anergic T cells. U.S. Pat. No. 5,747,299.

Enhancement of Adoptive Immunotherapy and Vaccines

The compositions of this invention are useful in conjunction with expanded populations of antigen-specific immune effector cells which are further useful in adoptive immunotherapy regimes and as vaccines.

Adoptive immunotherapy methods involve, in one aspect, administering to a subject a substantially pure population of educated, antigen-specific immune effector cells made by culturing naïve immune effector cells with APCs as described above. Preferably, the APCs are dendritic cells.

In one embodiment, the adoptive immunotherapy methods described herein are autologous. In this case, the APCs are made using parental cells isolated from a single subject. The expanded population also employs T cells isolated from that subject. Finally, the expanded population of antigen-specific cells is administered to the same patient.

It is to be understood that while the invention has been described in conjunction with the above embodiments, that the foregoing description and examples are intended to illustrate and not limit the scope of the invention. Other aspects, advantages and modifications within the scope of the invention will be apparent to those skilled in the art to which the invention pertains.

1. An isolated polynucleotide encoding a peptide selected from the group identified in Table 1 and the complement of said polynucleotide.

2. The isolated polynucleotide of claim 1 and a carrier.

3. The isolated polynucleotide of claim 1 and a pharmaceutically acceptable carrier.

4. A host cell comprising a polynucleotide of claim 1.

5. The host cell of claim 4, wherein the host cell is an antigen presenting cell and the peptide is presented in the context of an MHC molecule.

6. The host cell of claim 5, wherein the antigen presenting cell is a dendritic cell.

7. A composition comprising the host cell of any one of claims 4 to 6, and a carrier.

8. A composition comprising the host cell of claim 4 and a pharmaceutically acceptable carrier.

9. An isolated peptide selected from the group identified in Table 1.

10. A host cell comprising the peptide of claim 9.

11. A composition comprising the peptide of claim 9 and a carrier.

12. A composition comprising the host cell of claim 10 and a carrier.

13. An antibody that specifically recognizes and binds the peptide of claim 9.

14. A composition comprising the antibody of claim 13 and a carrier.

15. An immune effector cell raised in the presence and at the expense of a host cell of claim 5.

16. A method for eliciting a cytolytic response in a subject comprising administering to said subject an effective amount of a composition of any one of claims 3, 8, 11, 12 or 14.


SEQUENCE LISTING (64 sequences)

SEQ ID NO: 1 - DNA, 10 bp, Homo sapiens
gtggtatggc

SEQ ID NO: 2 - protein, 206 aa, Homo sapiens
MRWARLSALLCQRRVVWLSYGVASSYVLADAIDKGKKAGEVPSPEAGRSARVTVAVVDTFVWQALASVAIPGFTINRVCAASLYVLGTATRWPLAVRKWTTTALGLLTIPIIIHPIDRSVDFLLDSSLRKSTQQWGSPAPPDHTLVPGLCIGLLLHVNLLLLPGNVDTWLPGVQRPWHLGGFELDRSLETKASRSSGCRESQKGRT

SEQ ID NO: 3 - DNA, 1294 bp, Homo sapiens
acgtgacgaaaaccagtctgtaaaacccgagcctgggagaggggcttcggtgcgcggggggaatttgcagacgctccctgctggcggagatttcctgacctgtccttcggcgcgggactttcggcgggtcccggccggcagacccaagtgccggcggcggatgactgcagtggagccagtaccggctgtagtggccggggcccgtggcgggagagtcatgtcagagccgcagccgcggggcgcaggagcgagatcgtctaccgggacacgtgggtgcgatacctgggctatgcaatgaggtgggcgaggctttccgctctcttgtgccagcggcgggtggtgtggctgagctatggcgtggccagctcctacgtgctggcggatgccattgacaaaggcaagaaggctggagaggtgcccagccctgaagcaggccgcagcgccagggtgactgtggctgtggtggacacctttgtatggcaggctctagcctctgtggccattccgggcttcaccatcaaccgcgtgtgtgctgcctctctctatgtcctgggcactgccacccgctggcccctggctgtccgcaagtggaccaccaccgcgcttgggctgttgaccatccccatcattatccaccccattgacaggtcggtggatttcctcctggactccagcctgcgcaagtctacccaacagtggggaagcccagctcctcctgatcatactctggtacctggcctgtgcatcggcctcctgcttcatgtcaacctcctactcctgccagggaatgtggacacctggctccctggtgtccaaagaccctggcacctgggtgggtttgagctggacagaagcttagagacaaaggcttcaagaagcagtggctgcagggagtcacagaagggcaggacctgaacgctgtctgcttccctggaatccaagatgctgagtggaagtggaccctgggtgggcccggccctgtctttttcaggaaaattacatcctcccatggaggatgagagactgaggctcagggagggcaaggaataggcccaagatcacttggcaagctgggcacccaggacccccaggtgcttgacagagtcaccccatggtggtatggctgaacaaggagcggcagacaactcagggagaaactcaggagtgcagtaccagggacacctcaggacagattctctggccaggcccttccctgacccaataaatcctgaagaggtaaaaaaaaaaaaaaaaaaaaaaaaaaaaaaaaaaaaaaaaaaaaaaaaaaaaaaaa

SEQ ID NO: 4 - DNA, 10 bp, Homo sapiens
ctaataaact

SEQ ID NO: 5 - protein, 283 aa, Homo sapiens
MTSGPGGPAAAAGGRKENHQWYVCNREKLCESLQAVFVQSYLDQGTQIFLNNSIEKSGWLFIQLYHSFVSSVFSLFMSRTSINGLLGRGSMFVFSPDQFQRLLKINPDWKTHRLLDLGAGDGEVTKIMSPHFEEIYATELSETMIWQLQKKKYRVLGINEWQNTGFQYDVISCLNLLDRCDQPLTLLKDIRSVLEPTRGRVILALVLPFHPYVENVGGKWEKPSEILEIKGQNWEEQVNSLPEVFRKAGFVIEAFTRLPYLCEGDMYNDYYVLDDAVFVLKPV

SEQ ID NO: 6 - DNA, 3163 bp, Homo sapiens
cctttgccctgaagggggctggatgggcaaggcggccgcgatggctcgagctcgggcggtggcggcggtggccggaggcggcggtgcctcctcctcctcgccccggcgccggcggtgatccgagcgagcggccgcggcccccgatgagactgctggcgggctggctgtgcctgagcctggcgtccgtgtggctggcgcggaggatgtggacgctgcggagcccgctcacccgctccctgtacgtgaacatgactagcggcccgggtgggccggcggcggccgcgggcggcaggaaggagaaccaccagtggtatgtgtgcaacagagagaaattatgcgaatcactccaggctgtctttgttcagagttaccttgatcaaggaacacagatcttcttaaacaacagcattgagaaatcgggctggctatttatccaattatatcattcttttgtgtcatctgtttttagcctgtttatgtctagaacatctatcaatgggttgctaggaagaggctcaatgtttgtgttttcaccagatcagtttcagagactgcttaaaattaatccagactggaaaacccacagacttcttgatttaggtgctggagatggagaagtcacaaaaatcatgagccctcattttgaagaaatctatgccactgagctttctgaaactatgatatggcagcttcagaaaaagaaatacagagtccttggtataaatgaatggcagaatacggggttccagtatgatgtcatcagctgcctgaacttgctggaccgctgtgatcagcccctgactttgttaaaagatatcagaagtgtcttggagccaactagaggcagggtcatccttgcccttgtcctcccctttcatccctatgtggaaaacgtaggtggcaagtgggagaaaccatcagaaattttggaaatcaaaggacagaactgggaagaacaagtgaatagtctgcctgaagttttcagaaaagctggttttgttatcgaagctttcaccagactaccatacctgtgtgaaggcgacatgtataatgactactacgttctggatgacgctgtctttgttctcaaaccagtataaacacgtggaggtcgaagtcttcagagtccgcaccctccgggatgtgcccttggaagagggtctgtgttcacaattacgtgaagggaggacccttggggaccgccattctaaatatcatgtaggaatttaaaaagccaaaatactaattatttctttgtagtgtgtaaaggaatgtttttaaaagacaaaaacccaactctttgtggatttttatcaactctttactcagagccactctccaatgcaggtcacactccaattatgatggaagatattttttatacttaattgcagtagggactcattcccagacaaagcaatagtcacgacttcatggaaccaatcaatggattgttttttgaagactggcaataaagctgtccattcaattccaaatactggttttaaggtatagccactgatattctttcatgtttagaaattctttctgttattattcaagaaaatgtttttaatcatgctaataaacttttttggagatgactttggcatcatgtttgaattcatataaagctcccctagcattttttattggtttggcttcaggagtacccaaatagtagcattatgagaatgacgcagacaatttgaataggggggaaggaaggcttcagacttgggggaaggggagattattgcaaattgcagtgaacactgagtcagtaaaaaaaaaaaacagaaacaaaaacccagcctcattcagatacagaacttcagggacccctccccccacccccccagttaatgctgctgtgaaaaatgcaaaataacctggttctgactttgtgatcactcatgtcccatacactgaactttgtttttttctggacaatctcaggttctcagaattgaaacattcagttttgtctactgacaaaatgcaactaaaaatgttttaattcaacttcttactctacacttatatacctctctcccagagcttggctcttccctgaaatccttaaaggagtttattatttgccaagaatatggtttgatgaagaccgtgtcagactcatgcattctggaatccgtgagtgcctccaaccataaacccagaaatgctgccatagaggaacccattagcaagatcaataaccatcgtggatattccaaagagcagagcatttaacccagaatctgaaatctcagtaataacagaaatgtcaatatgagattgtggttattaacaatatgttatcctcactactttatttcttttttcatccaggtgtttcattaagtattctcatttatatgctcatagaagtcatcagatcaacacattcactccaacacctgggtataaaggcgaattaaaacctgaagaattactttattattcattaagaactgcattcccacatcctcacaccaatatcttccactgttactgtgcagtcataataagagttgggttatgtgatttatgcaaaattgttattggtgtgttctaatgatttttttccaggaattcaagtaagtgaagtacaaatacttagcaccatagattgtaaaaatagaaagaggaaatgagaaaacttaatgtcgttcttgtacagttgagttttgcttctttataaactgtctgttaaaatcaggggttgctgttagactgtcaagcagaagtgatggatgttcaaggaggcaggttgccccaaagtgttttctaactttcaagtagagaactttatgctctaataagaaaaatacaactcattcttaaccacccccccaaaacagagaaaacacatacatttatgcattttaaagataaaaagtagtttctcaaaaaaatctgaagttctctgaaaaccagcttattaaaagtgctgttttgtggtgaaaattgaaaccaaatccactcacatttcctatcgggtcttgaaattcttggtggtgtgtcaccattccactagatggcagtgttgcttactgagtgtcaatggcttttttccccgtaaatggaatcatgtttttctcccccaaagtacaataaagctgccttgtctgcaccaaaaacaaaaaaaaaaaaaaaaaaa

SEQ ID NO: 7 - DNA, 10 bp, Homo sapiens
gctccctacg

SEQ ID NO: 8 - protein, 229 aa, Homo sapiens
MTPQLLLALVLWASCPPCSGRKGPPAALTLPRVQCRASRYPIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGK

SEQ ID NO: 9 - DNA, 1161 bp, Homo sapiens
gaattccgcagccatgaccccgcagcttctcctggcccttgtcctctgggccagctgcccgccctgcagtggaaggaaagggcccccagcagctctgacactgccccgggtgcaatgccgagcctctcggtacccgatcgccgtggattgctcctggaccctgccgcctgctccaaactccaccagccccgtgtccttcattgccacgtacaggctcggcatggctgcccggggccacagctggccctgcctgcagcagacgccaacgtccaccagctgcaccatcacggatgtccagctgttctccatggctccctacgtgctcaatgtcaccgccgtccacccctggggctccagcagcagcttcgtgcctttcataacagagcacatcatcaagcccgaccctccagaaggcgtgcgcctaagccccctcgctgagcgccacgtacaggtgcagtgggagcctcccgggtcctggcccttcccagagatcttctcactgaagtactggatccgttacaagcgtcagggagctgcgcgcttccaccgggtggggcccattgaagccacgtccttcatcctcagggctgtgcggccccgagccaggtactacgtccaagtggcggctcaggacctcacagactacggggaactgagtgactggagtctccccgccactgccacaatgagcctgggcaagtagcaagggcttcccgctgcctccagacagcacctgggtcctcgccaccctaagccccgggacacctgttggagggcggatgggatctgcctagcctgggctggagtccttgctttgctgctgctgagctgccgggcaacctcagatgaccgacttttccctttgagcctcagtttctctagctgagaaatggagatgtactactctctcctttacctttacctttaccacagtgcagggctgactgaactgtcactgtgagatattttttattgtttaattagaaaagaattgttgttgggctgggcgcagtggatcgcacctgtaatcccagtcactgggaagccgacgtgggtgggtagcttgaggccaggagctcgaaaccagtccgggccacacagcaagaccccatctctaaaaaattaatataaatataaaataaaaaaaaaaaaaaggaattc

SEQ ID NO: 10 - DNA, 10 bp, Homo sapiens
tgtattttga

SEQ ID NO: 11 - protein, 227 aa, Homo sapiens
MRVAGAAKLVVAVAVFLLTFYVISQVFEIKMDASLGNLFARSALDTAARSTKPPRYKCGISKACPEKHFAFKMASGAANVVGPKICLEDNVLMSGVKNNVGRGINVALANGKTGEVLDTKYFDMWGGDVAPFIEFLKAIQDGTIVLMGTYDDGATKLNDEARRLIADLGSTSITNLGFRDNWVFCGGKGIKTKSPFEQHIKNNKDTNKYEGWPEVVEMEGCIPQKQD

SEQ ID NO: 12 - DNA, 2475 bp, Homo sapiens
ccggaggagtccgagaggaagcggaggcgcgagctggaggcggcggctcccgtcggcctccggcaggactgagcgctgggaggccggaaggcgggcgcgcacggcggagaggcgggcgggaggccggagcatattaatgaaaagtgccataaactgaaaaaccaaacatgagggtagcaggtgctgcaaagttggtggtagctgtggcagtgtttttactgacattttatgttatttctcaagtatttgaaataaaaatggatgcaagtttaggaaatctatttgcaagatcagcattggacacagctgcacgttctacaaagcctcccagatataagtgtgggatctcaaaagcttgccctgagaagcattttgcttttaaaatggcaagtggagcagccaacgtggtgggacccaaaatctgcctggaagataatgttttaatgagtggtgttaagaataatgttggaagagggatcaatgttgccttggcaaatggaaaaacaggagaagtattagacactaaatattttgacatgtggggaggagatgtggcaccatttattgagtttctgaaggccatacaagatggaacaatagttttaatgggaacatacgatgatggagcaaccaaactcaatgatgaggcacggcggctcattgctgatttggggagcacatctattactaatcttggttttagagacaactgggtcttctgtggtgggaagggcattaagacaaaaagcccttttgaacagcacataaagaacaataaggatacaaacaaatatgaaggatggcctgaagttgtagaaatggaaggatgcatcccccagaagcaagactaatggaaatgtggagagaattgaagaaagcgcactttcactcttaatgggagagctataaatggcagagctatgtgtaaatattttaagagcatgcagccatcttggtgtgtgcatgagtattgtctcttttgatatcaggattatttattgctaacgtaaatagatagcattgtaaataatcatcacaatgatcaaatcactgaaccatgtctccgcacatttccctaaaagtacaatgtttagactgctatggtaatacatattttaaattctaaaagcatacacaatgtgtaactgaatggtttgtgaaaaatatattgatatatatactagttgctatgaaaatatcatggaataatagggattttagggtggatactttattttcttttatgtttctatatgttgcgttgtgatgacattatcttttaaattaaaaagagatttggctagttgtgtgtgtaatgttactttacagtccgactctcctgatgtacctcttttcatgatctttttctttccttcccaagaaactgaggaatgtttaatatgaaaacatacatcggatatgtgaaaagcacaacaaaattcttaatgtacacagtaaaaaagtaaatatataaatgtagatggcatttaggaccacagcttgctggatttgtgttagctatgggaataacttgattttgtataagctatttagagtgaggctggaggtggcagcttcacagaactggagaaccaggccaagtcccctccccaacctaattaggtcattcaggacagctaagtcagtatatttagagcaatactagcatacgtttttcttaattgttatcagcattgaccaagtggtttggaaggaggcatgctttaatatcacaataattttgatttgtaaaccaagaaattaatcctgtgtttatctaacttcataatagcaattattgcccgaagctatagtggcatatttacaaaagttcttattactgggcggactgataacatttaaaaaataattgtgtttgaccccaaatgactttatacccaattctacataaaaatatagaagatctatctttttttgttaccttcagatgttcactaaataactcagtttttaagcagaagttttcagggcattaaatatatgttgtgtatgaagtatctcaaactggaacataaatttagtgatcaaactgccattcacagtgtaaggcagcacttaaatttcgaacctaaagtttagatgcattgtataaaaaaacctaaaagcagtatctgttatttagctgtaaaccaagttggaagctattcggataatttcttaaatattgatgaactttggagtactgtttcttccttcaaactgaatgtaattaattcatgaataaatgcaccttatatgtttaaacaatctttgtatacttttgggatttttggtgcttatatgctaaatcacattcagcatgtgtattttgacatttaaaatacttccctcaattctgtaaattaaaagaatagttattttacagttccagggattgtgaaataaatgttgcagttttttaaaataatgaaaataaatactcttggttttgctttgtgaaaaaaaaaaaaaaaa

SEQ ID NO: 13 - DNA, 10 bp, Homo sapiens
gtccccccac

SEQ ID NO: 14 - protein, 85 aa, Homo sapiens
MIYVSTGAWGWAGSCFWQWSFCPPACVGCIEEEAQGSSEQELHYASLQRLPVPSSEGPDLRGRDKRGTKEDPRADYACIAENKPT

SEQ ID NO: 15 - DNA, 6853 bp, Homo sapiens
aagcttctgaagggtttagatgtaaaatgtactgatatttgtaattttaaaagatatttaaaagtgagatggattgataaatatgtgataaagcaaataaaaaatgttaatagagccaggtgcagtggcccactcctgtaattccagcactttggaaggctaaggtggaaagattgcttgagaccaggagttcaaaatcagcctgggcaacatagtaagaccccatttctacaaagcctcatgtggtagctggtgtctgtagtcctagctactcagaaggctaaggtgggaggacctctgagcccaggaattcaattctgcagtgagctatgatttcaccactgcacttcaggctgagtgacagagtgagaccccatctcaaaaacaaaacaaaacaaaaaatgttaatagtagcatctaggtggtaagaatatgttcactgtacaattattctcatttcaccctatgtttgcactttttaataataaaatgtaaaaaaaacaaaacaaacaaacaaaaaaccctgaatattattcagcatggggaacatggaggatggggagaagggtgggggaggaagtagaaggttcttgaatttggaaggggaaacgcaaattaatatggacccacccaggcaccacatctcctcctcaccccttgccttacaggcgctccccagtcttcaccctcctcaaggagtgggtgtgcaatcctccagcacccatctccttctccatcacagtgccactaagaagccttcacccaggtctctccagagagcctcaggccgctgcctttacttagttctgtgttcaatgccagaatgctgcctcctacaggaagtccacctgtattgcccacacctcctttcctgtcaccaacttgtcaccaactttctgtccttgatctatccacagggctcatgtagatctagtatggctgcctttaactctcatgtttgttaatcagacagccaagcagcctgctgcatagagctgcagaacaccaaatatgccagagctcccagtctgaactggagcagggtacatgtgtccacagacatatgccaagatcaagaggtctcaacagatgcagtgtaagaggtaatagagaagagttaatcaaggaagacacctgaaggtggtgggtgtttgctgactagtggcaggatcagtgaaatgactggagctgaggcagattatggccctagctacaggcccagaagtttgaaaagaaagatgttgtaaccctaaccctggagccgaacttcctctcctaacaatgctggggaggaacccaggctgggggagaagttaaagccagaggaggggcaggaatgtctgaggtggcaacacttctcttcagccagacagcactggccagtttggagtctgtccatcctgcaggccacaagctctgggtaagctgggaatgggcagggaccttggtggaaggatggtcacaccccagagtggggtgaagctaagatgaggggagggagagtatgggtttgagtttccctgggccgtcgaggaatcctctgagtctctgctccccaaagaaattaaagacaattcatttctgtgcccacggcccttatggctccacctgcacttctgctccccaccccccagaattcctcttaaacccagaaggtcccagtttccagaccctagtcagtatatctggctctggggtgaagagaacggccccctcttcaccctcaaacaggaaccagtggttggaggggaggaagtgcctgaggggaagttatggggccccagatactcctccatgccccacttcagccctagcagcatctgcctgtgggaagcagctctccacaccagccaagggggcccccacactcccgcgctgctctgcggctcagggagcagcccacctgctgggtgtgctgatatcaccctcccttcttccccccagtgcccacacccacccaggcccaggctccttcccctccatcatccccttaccagcacctagaaccatccagggctgaaaagtcccctccaaaccacgtggtcagcccagggcagaggaaagggctgggctctggagttgggcagagctggccttaaaccccagctccacctttctgggatgggtgacctagtaaagtccaggcttgaatctcgggtctttacttgggcaacgggcaccatgataccctatgttctggggattagcagtgaggaatggaaagtgcccagctcgggttggcacataagggaggctccccagcctgggaacgattataacagagggcccctcacttcacagatgaggaacttgaggcaagtcaccagcccctgatcatttcgcctaaaagagcaaggactagagttcctgacctccaggccagtccctgatccctgacctaatgttatcgcggaatgatggtaagtaaagtgtctcttgcatctgcatagagagagtcctgggagcttaggaagtgatggggaacagtgatgtatgcagctcatgactaggtggacaggcctctggggacagctggtacaggagggaaagggacctcacgggaggcccagaaacctggtaagaggtgaggtattaaggtctgggatggagaagctctgagggtatatttttctgcctctaaaactgttggagagggaatctgagaaagctgcaaccaaccaggaggctggggtacgctggagaaggaatgggcttcctaaccttgagccctcttccctgaagatatatgtatctacgggggcctggggctgggcgggctcctgcttctggcagtggtccttctgtccgcctgcctgtgttggctgcatcgaagaggtgagcgctgcactccctccctccccctgcagcagtgccccctgtgcccccacccccacacgctttcccactgctttcccagaacactgcctggccctggagccactgggaagccaacaggggagtccacgcctgctggtggggggagcccgggaggcgggagaagcacaaagggtgggctgtgttgagcttcttcttttcttccagtaaagaggctggagaggagctgggtgagtctggggacagggaagggggagggcaagagagatcctgagtgggtgagtggggagaagcatggctgagcgctgagaggagggttggggacgggagacaaggagagagaaagtaggagcatgagagaggcagagaaaatcgaggcaaaagagaaagagaaaatgagacagaaaccaagagaaaaagtgagacagaggataggagagacagggagaaaatgagagtgagagagacacaaagagaagagcaatgaaagagagagagagagagaggctccagaaccaggcacagtggctcacgtctgtcattccactatcgcaaggctgaggcaggaagatagcttgagctcaggggttgaagacaatcctggacaacatagtggactctgtctccaaagaaaaaagagagagagagagagagagagagagagggagagagagagagagagagggagagaagtaagaaaggctggaggtgggagcagaactcacagggaaggatctgacgcatcgcctcccatcagcaccttctgtcctggtcccaggcccagggctcctcagagcaggaactccactatgcatctctgcagaggctgccagtgcccagcagtgagggacctgacctcaggggcagagacaagagaggcaccaaggaggatccaagagctgactatgcctgcattgctgagaacaaacccacctgagcaccccagacaccttcctcaacccaggcgggtggacagggtccccctgtggtccagccagtaaaaaccatggtccccccacttctgtgtctcagtcctctcagtccatctcgagcctccgttcaaattgatcatcatcaaaacttatgtggctttttgacctttgaatagggaattttttaaattttttaaaaattaaaataaaaaaaacacatggctcacccttccacccactctggggtcaaatagtaatttgttgggtgaatgacagtgttcagggacccaagctcccctaacagccagaagagggtatgtgtgggcctggcaggaaagggcagttgccaaggaggagtcatatctgatccttcccatttctcaggacaatcaggctcagcctcctgggactgggggaagcagatgtgctgagctcccacatggtggtgggaggggcgctgggaccacagccggcagctgccttcttggacctttccaggtcagacctggtggaagggaaagttcagagttgggggaatccggagagagtagatttggcatctggagaatggagaagaaaacacttgagactcatgaggagttagtggtggggcagatttattggggtcttttgaagaggactagggacatctgggctctggaatcactcctcggggcccatctgaggagtggcagtgtgttcccatgtgacagtggcctggtcagagagaggacaggagctgctcagtgttgcagtcccgaggctctcctcttcctggtctctgtcctccctcctcccactctcttactgcccctcccatcccgtccactattgcccctggctccattactcacatttgccctggtaatagacggtgctgcccacggccacagagagaaagctgacagcatagaatccagcccgaaggaggaggactgtaccagcccctagctgaggatgttctgcatggggcaatggagacgggggttggggaagaagtgcacacaggctcagggagggaaggggcctcagaggagcatccctgcctcccaaggacattgcctcttggggcctccagccaggaggagacaccacctcccagcatctcacctttctccaccaccagccgagtcccattccctgtcccgacaccaaggcccagcacctccactctgcacacgtagatgctggcgtcatggcctcgcacgtcccggatgtgcagctcagcctggtggtcatggaggaaacgggaagaagcaagtggggccaggcggcccctgaactctggggttccattcctcacctccttccctggaaccacctcatctcggaaccacgtgacggagccaatggccagtctcccttggctggcattgaaggagcagggcaggaaggcagaggatccttccagggtacgaatctcagggggctgggacacccagagagcacaggaatcctggggggaaaagggagacccagagaaacacctccccagttattccaaagagaaaagacaacagagcttggagtagaacatcccagctttctccaggcatagggtgcatgggaatagatactttggggcctcattaaacccttccctcttaaccaatctgatttcttaacattgcttattaaatcatttttcggctgggtgcagtcgcacgcctgtaatcccagcactttgggaggccgaggtgggcggatcaccaggtcaggagatcgagaccatcctggccaacatggtgaaaccccgtctctactaaaaaaatacaaaaattagccgggcatggtggtgtgcacctgtaatcccagctactcgggaggctgaggcaggagaatcgcttgaacccgggaggcagaggttgcagtgagccaagattgcgccattgcactccagcctgggcgacaaagcaagactccatctcaaaaaataaaaaataaaaatcatttttcaaattcttcctataccaactctcactctcaccctctgccatcattctccagccagttcagtagtaacttgtctagctgaaatgtaaaccatcatggtgaaattaagctcattaatgaattgcagctgcctagttaactaatatcactcattatattatgcaggtattattttagtacaaatggcattgtacagtaagccatccttcctctttttcttttttctttttttgagatggggtcttgctctgttgcccaggctggaatgcagtggtgcaatcttggctcactgcaaactccgtcccctgggttcaagcgatcctggtgcctcagcctcccaagtagctgggactacaggcacccaccaccacgactggctaatttttgtattttcagtcgagacagggtttccaccatctggtctcaaactcctgacctcaagtgatccacccacctcggaccaggctggctcaaactcctgatctcaagtgatccacctgcctcggcctcccaaagtgccacccagccactcttggttttcgttaaagaaagtaactaattaaatctccaggtgaagacgtggccttaattggttgagattcctatttaacccgtccatgttgatgaattaaaccaaatattaaaatccctgattaaattatctacttagggaaatttacaagtcattctatttcagtggttctcaaacttgagtgtgtatggaaattacctggagcatctgctagaacagattcctgggcctaccccccgagtttttgactcagtaggtctggagtggggcctaagaatttgttctaggttcccagaaatccacattttgagaactcctgcatttagttaataatatgcctgatagttaaggtctctcagttcattaaaaacagtttcggccgggtgcagttcgcacgcctataatcccaacactttgggaggccaaggcgagtggatcacctgaggtcaggagtttgagaccagcctggccaacatggtgaaacctcgtctctactaaaaatacacaagttagccagcagtaatggcatgcacctgtaatcctagctacttgggaggctgagacaggagaatcatttttacccaggaggtggaggctgcag

SEQ ID NO: 16 - DNA, 10 bp, Homo sapiens
accttgaagt

SEQ ID NO: 17 - protein, 277 aa, Homo sapiens
MIILIYLFLLLWEDTQGWGFKDGIFHNSIWLERAAGVYHREARSGKYKLTYAEAKAVCEFEGGHLATYKQLEAARKIGFHVCAAGWMAKGRVGYPIVKPGPNCGFGKTGIIDYGIRLNRSERWDAYCYNPHAKECGGVFTDPKQIFKSPGFPNEYEDNQICYWHIRLKYGQRIHLSFLDFDLEDDPGCLADYVEIYDSYDDVHGFVGRYCGDELPDDIISTGNVMTLKFLSDASVTAGGFQIKYVAMDPVSKSSQGKNTSTTSTGNKNFLAGRFSHL

SEQ ID NO: 18 - DNA, 10 bp, Homo sapiens
ggaccgaggg

SEQ ID NO: 19 - protein, 194 aa, Homo sapiens
MRLLPCAAGTSAFLSSQLTIGASSSSRPSTPAWFSVLEYIQPFRSCSFLLSSSPPGALPCSLCTSDSILPVSDDFTSSKRPLGPAFSKAFLENAGQDLPFNFVFSVCSVSSAGCLSSRITEDMSVLFPLMPGARLGHVCSAMANVRRQGWSHSGLLALWPSGRSKGRSCQKETCLVTGRSQGFQNPIQVIPSWW

SEQ ID NO: 20 - DNA, 1841 bp, Homo sapiens
tgttttgtttctttatttggacttaacggccactcatttctgttggcgccatgcgactgttgccgtgtgcagcagggacctctgccttcctctcctcccagctcacaataggtgcctcttcctcctcccggccatccacgccggcctggttttcggttctggaatacatccaaccctttcgttcgtgttcttttctcctgtcatcttctccgcctggagctcttccatgttctctttgcacaagcgactctatccttccagtttcagatgatttcacctcctcaaagaggcctttgggaccagccttttctaaagctttccttgagaatgctggtcaagatttaccttttaattttgttttttctgtttgttctgtctcctctgccggctgcttgagctccaggatcacagaggacatgagtgttctgttcccactgatgcctggtgccagactgggacacgtgtgctcagccatggctaatgtgcgcaggcagggatggtcacacagtggcctcttggcgctctggccgagtgggaggagcaaggggaggagctgtcagaaggagacctgcctagtcacaggcaggtcacagggcttccagaatcccattcaggtgatcccctcctggtggtaaacagcacacctcagaagtggtgttttgtcatagttgttcttatggctcccacagcctcatctctgttttgtccatttgggattgaggagttgttgagtccagcgtcaccttcagagccagtgcactccttatcaatagtgagggccgctcgttttgattgttccgtttcgcttgataaagctgagtgagaattatattggccaacagaaacgtctctctccatgtgaacacagccactgggtcaccagggacgtggcttacagttccagcaggctcctgtcacactggtgcctttgggcagtacagaactgtgctgctggtggtgacgtccggggctgtgcagtgtcgcttccttttgcatatgttgctattggtgtcatttcctctctgagatcagtggaagtctcaggtcagtcccgtggccctgtattgcttagtgggccatgtgtttaaacttccaaaaggcatattttaaatagttattggagtgttgatgtgaagagctcctttgataatgtattttctttgtagaaatttaaaagcctgttgattcaagaactttctaaagtatttccggaagacatggctaagtatcgaagcatccggggggaggatcacccgccttcttaaccagctcaccctccctgtgtgaagatcccctgggactgcgatgcggcgtgaggctgggactgcgagtgctgacgccaccttcctgctgaggtgggactgggccctggacacacccctcagcccctctgtcctcattgtttggcctcatgggaccgaggggctggaggagaggcggagctgtgccccagctgttccagcagcttgtctggcgtcaactggctttcagagtgctgacccctcatcactgtggggatcattctctctgagggcagatgaggcgcaggaaaatagtcttggaaatgttaaatatgatgggtaaattaaaagttttacaacattctacctaatatttttcttttaacatactctttctgttctattgtattatggtgtccgaaagctaaataacgactaggaaaaatttttttaaaaaaagaaaaatcagtttaatgtgggaagtacttaagtggtattatattttacattttcaagtatagtgcataaagaatgttttaaatgtaactgttttcatggatttcaattagacatgcctataataaactaagtatgtggctt

SEQ ID NO: 21 - DNA, 10 bp, Homo sapiens
gccggcttga

SEQ ID NO: 22 - protein, 439 aa, Homo sapiens
MTSILTVLICLGLSLDPRTHVQAGPLPKPTLWAEPGSVITQGSPVTLRCQGSLETQEYHLYREKKTALWITRIPQELVKKGQFPILSITWEHAGRYCCIYGSHTAGLSESSDPLELVVTGAYSKPTLSALPSPVVTSGGNVTIQCDSQVAFDGFILCKEGEDEHPQCLNSHSHARGSSRAIFSVGPVSPSRRWSYRCYGYDSRAPYVWSLPSDLLGLLVPGVSKKPSLSVQPGPVVAPGEKLTFQCGSDAGYDRFVLYKEWGRDFLQRPGRQPQAGLSQANFTLGPVSRSYGGQYTCSGAYNLSSEWSAPSDPLDILITGQIRARPFLSVRPGPTVASGENVTLLCQSQGGMHTFLLTKEGAADSPLRLKSKRQSHKYQAEFPMSPVTSAHAGTYRCYGSLSSNPYLLTHPSDPLELVVSGAAETLSPPQNKSDSKAGE

SEQ ID NO: 23 - DNA, 1571 bp, Homo sapiens
ctctgtcctgccagcaccgagggctcatccatccacagagcagtgcagtgggaggagacgccatgacctccatcctcacggtcctgatctgtctcgggctgagcctggaccccaggacccacgtgcaggcagggcccctccccaagcccaccctctgggctgagccaggctctgtgatcacccaagggagtcctgtgaccctcaggtgtcaggggagcctggagacgcaggagtaccatctatatagagaaaagaaaacagcactctggattacacggatcccacaggagcttgtgaagaagggccagttccccatcctatccatcacctgggaacatgcagggcggtattgctgtatctatggcagccacactgcaggcctctcagagagcagtgaccccctggagctggtggtgacaggagcctacagcaaacccaccctctcagctctgcccagccctgtggtgacctcaggagggaatgtgaccatccagtgtgactcacaggtggcatttgatggcttcattctgtgtaaggaaggagaagatgaacacccacaatgcctgaactcccattcccatgcccgtgggtcatcccgggccatcttctccgtgggccccgtgagcccaagtcgcaggtggtcgtacaggtgctatggttatgactcgcgcgctccctatgtgtggtctctacccagtgatctcctggggctcctggtcccaggtgtttctaagaagccatcactctcagtgcagccgggtcctgtcgtggcccctggggagaagctgaccttccagtgtggctctgatgccggctacgacagatttgttctgtacaaggagtggggacgtgacttcctccagcgccctggccggcagccccaggctgggctctcccaggccaacttcaccctgggccctgtgagccgctcctacgggggccagtacacatgctccggtgcatacaacctctcctccgagtggtcggcccccagcgaccccctggacatcctgatcacaggacagatccgtgccagacccttcctctccgtgcggccgggccccacagtggcctcaggagagaacgtgaccctgctgtgtcagtcacagggagggatgcacactttccttttgaccaaggagggggcagctgattccccgctgcgtctaaaatcaaagcgccaatctcataagtaccaggctgaattccccatgagtcctgtgacctcggcccacgcggggacctacaggtgctacggctcactcagctccaacccctacctgctgactcaccccagtgaccccctggagctcgtggtctcaggagcagctgagaccctcagcccaccacaaaacaagtccgactccaaggctggtgagtgaggagatgcttgccgtgatgacgctgggcacagagggtcaggtcctgtcaagaggagctgggtgtcctgggtggacatttgaagaattatattcattccaacttgaagaattattcaacacctttaacaatgtatatgtgaagtactttattctttcatattttaaaaataaaagataattatccatgaa

SEQ ID NO: 24 - DNA, 10 bp, Homo sapiens
ttcactgtaa

SEQ ID NO: 25 - protein, 298 aa, Homo sapiens
MKTLQSTLLLLLLVPLIKPAPPTQQDSRIIYDYGTDNFEESIFSQDYEDKYLDGKNIKEKETVIIPNEKSLQLQKDEAITPLPPKKENDEMPTCLLCVCLSGSVYCEEVDIDAVPPLPKESAYLYARFNKIKKLTAKDFADIPNLRRLDFTGNLIEDIEDGTFSKLSLLEELSLAENQLLKLPVLPPKLTLFNAKYNKIKSRGIKANAFKKLNNLTFLYLDHNALESVPLNLPESLRVIHLQFNNIASITDDTFCKANDTSYIRDRIEEIRLEGNPIVLGKHPNSFICLKRLPIGSYF

SEQ ID NO: 26 - DNA, 10 bp, Homo sapiens
aattctgtaa

SEQ ID NO: 27 - protein, 305 aa, Homo sapiens
MAAGLARLLLLLGLSAGGPAPAGAAKMKVVEEPNAFGVNNPFLPQASRLQAKRDPSPVSGPVHLFRLSGKCFSLVESTYKYEFCPFHNVTQHEQTFRWNAYSGILGIWHEWEIANNTFTGMWMRDGDACRSRSRQSKVELACGKSNRLAHVSEPSTCVYALTFETPLVCHPHALLVYPTLPEALQRQWDQVEQDLADELITPQGHEKLLRTLFEDAGYLKTPEENEPTQLEGGPDSLGFETLENCRKAHKELSKEIKRLKGLLTQHGIPYTRPTETSNLEHLGHETPRAKSPEQLRGDPGLRGSL

SEQ ID NO: 28 - DNA, 37476 bp, Homo sapiens
gatcaccatggcaaccggcctgtgtttgttccaacaggtgggcagaacctggtgcaagaccgcagaggcaggggcgtcactcccagacccggtctccagacccaccagcccctcaggagaacactgctacagagtggggctgagggcccaggccccctcccctgcttcccccaccgcgtccctgtcccagccagtccctgcatttcctgcaagctccctgccctcccccaccggcctggctgtgctgggtcaggatccggcagagcctgggccgagcgtcccgaaggagagcccacccaggggtcctgcccccgatcctgcacagacaggtgtggggcgggcatggatttgagtgagttgggatccagaagccaagacagttgttggcagacgggccccctccctttgaagagctgtgttctggaagcctgtctggccttgggctgcccaggtgcccttcgggtgaaaactccaaagtctgaccaccccccaccccttgtccccaaagggctgcgagtgtccagctgtattcgaccaagggcaggtgcgaccggggggccgctgcgccctcggcaagtctgagagcctcaggcccccgacaggaccatgcttagcacggacttctcaaagtgtggtccctggactggcggcagcagactggagctgggaagtgcaggcgctggctggcgtggggaccacacctgtgatctcagcgccttaggaggccggggaagggggatcccttgagcctgggagtttgagaccagcctgggcaacatagggagacccccatctccacaaaagaaaccaaaaaacattagccggcgggtggtcccagctactggggaggctgaagcgggaggaccacttgagccggagagttcgaggttgcagcgagtggagatggtgccactgcgctccaacctggttgagagctatttttgagagctttttcgagactctgtctgaaaaaaaaagggaaaaaacgcagatcgcgcccccacccagacttcgggaccagcgggttggggacaggacccatcaatgccttaacaaaccctgcaggagaccgcgggcccgctggggttccaggacagcgcagcggaggaagagcggggagccacccgggaggggctggcggggtccagtgcagaccccaggggctccgggggctctggccggctgtgaccccacaagcgccaactgggccctcaccagagccccagggacgatcgccccacagaccgtccgcgtgggcgtggggcgtcccgagtggatccctgcaggacgcatccccgccccgagaagagagcggcggggcccgcgaggtgtccctgagtcaggcccttccagaaaggtctgcaccgctgctggtcttcgtgaccgtggcttccctggggtccgcggaggagaccccgccgaccacccccttctagaccccacgcgccacggggagggcgcgcgcggccaggcggggtcccgaggcagccaagcccgctccccgtcccgcagccacctgtgggttgactcacagccccgcatcccgggggagggggctccggcccggctcaaggcacggagaccccggccgggtcccctaaggcgccgtggacgcggggggcggggcgagcgcggtcaggccccgccccggtcaggcccggccccgcccccagtcagaccccgcccccgcagcggcgtccgcccagcagttgctcagtcgtcggcgcgcgcggctgggagcggtagctgtgcctcggcgtccccgagctggtgccgagcgcagcgccccaggagcccccccatccccgcgccggcccacggtaagtgccgcgggctgcggccagtgctgttgggccgccgtctgggctcggccccgcgccccaacccggacggaccgacggccgccgcccccttggtccgggtcccccaggaggcacgggggtggcctccgaggcggggggcggtgggaaggtctcgacgtcccctcggccctggggaaactgaggccgcgcatgcttggaggggcggggtctcgggggcatttcccctccgaacgcgcaggagagggtgcggcggtggggtccgcactgcggagcggacggggagggtatccgggcgctggcgacggcctcccccgcacttgagccgccaagcgaggcttcgacccccgcgcctccctccccgggggagcagacgggcccggggcctcccagcacccctcccgggcgggctgcaccccccagcgtcctcgtcccttccctcctcccgcaggtcctcgtaaggccctttagtccccccgaaatccctgcgagtgcaaagcggcccctccttcagccccactcggcaaagggctgtgggctcttgcctgggccggggtctccgggggaggagtttaaagtgacacactagacccgcccccagactcaccccggaccgcccgccccctccgaggggagagacccccatagaactgggggtgtggctcctgcgggcgcccggggccccgggaaagggggtgccgcgaggcggggcagggagggggtcggtgagcagagggaggagggagcgctgcgcagacacgggcgaggggaggaagaaggcgcctcgggctccggtctcctgcgctcctgctgggcgccagcggctggaccttggcagccgtctgaggcttcggggagcccaaggccaccggcagcgcttgttagaatgcagattcccggggttcgactggaatgagaccccggggagcagcgtttgcagcgggcccgccaggtgacctgcacctgggcaccgccatcggcttcgcaggggccatctggaggagtcggtgagaccagggagagattcctgcgggaaacccccagggcctggtgaaggggagtcggggggctgagggaggaagagcagggaagctggagagaagaccaagagggcagcgtggagagccagcctgtggggcagatgtcctgccacctcccgccccaggcacacacccagggcaccatcagctaaccccctgccccaaatgtggtaagaagggcgcccccaacacgttgaaggcgtttgttcctagacagcctgcactggggaggaaggctgacccagcctctgctccgctgcccccagactcagctgcctcactgcccccaaccctgagcccccaaatggcctggcaggagacagcttggcttaagagtcccaggttcacatctgggctctgggcaagtcgcccccaccctgggctgtgaaatgagatgtgccatcctccttgcaaaatgtcattccccattcagcccaggcctgggggacagccagggagcaaggtgggatgggcaggtgtggctcagccccagtgccacaaggcaggagctccttggcggtcctccctctgctcactggacagatgggagtgtggttctggtcctggcatcaggcatgagcagagtatgggggggttcatcctgcctcatgctactgtcccagggtgagagactgggaaggagttacatcacgtctgggcccacgggaaagacggggggtcccctgcacctggctcactcctgccccgtaagtgctgagagccacccaggcagccagtgcttcctgtactggtcacagacagatgcatatgcccaccattcacagcagctccggcctctgtggcaggtggctcccagggcccacgcagttggctgcagccaggctatcagctgcccaggaagcctggggtttccaggggaaccaaaggggtttctgcgcacccaggatgagccaagtcttctgtacacctgtgccaagacgccattattgcacccatcctagcaacgggcagtgggaggggcaggagctgcagttggggtcaggggggctggcctagctggttgccccaggagcttagcactggggacaccaaagagaccttgactgccgtccccagcccacttgctggctctgggaccttgggcgggctcccagccctgcgcctccgtttgcccatctgagatgcagggagcaaagggcacccctctcgggccgctctggggatgaggaggtgaatgtggagaggaaaatgcagggagggtaccgagccccctttttctgcctggaacatcttgttatcacaaagacaccccctccccaaccccgcagttcaggccacttggctcctgcgtcacatttgtctccaggactgcgagcccccatcgcccctgtcacacgcacatgccgcggcggttgccctggcaacagctccagcagcggctgctccactggcttcgggggacagcagggggagcagcctcttccacccagcaagggaggggaagggggcttgtgactcccaggcccctgaggatttcacgactggctgtgggggtgacagcttccagaagcactgtacccagcctaccaagcccccccatcttttgggggggaggctcacaggggaaggcactgataggtttgaaggaggtgagtcccccccgcaagcattgcctctcacagggtgggagacgcaaaccctccttcctgtcctcagctctagggtctggtcccgtaagagcctgcacccagggctcgggggctggcagggaccctggaatcaagacgagatatccgggaatggtgtctgagctggtgtgggtatagcttggggctggtgaccctgaacccccacagacacagcaggggcccgggagctggctcgacaaagtccgtttggaacccaccactggcatggtgtgctcaggcccccacgctgcccactgagcttatgagtctagtgccactgtggggagctggcctggcatgcagggccaactgtccgtgagagcctgagtgggcggtagaacctgctggtcttcgtttcctctcctgtcccgtgagaatgactatgccctttgcgagggccagtgtgcccagcccggaggcaccctccgcaggtggatgagggagcactggcaagggaagggacccagacagcaaggccaggaccccggggggctgggggagcgcggtgtcctagaaatcagaggccggcaaggctgggacaggaacccaagagtgggtacctctcttgtggtggggcagaggtggcctggctctgggacagtttccatggagcggggaggagacctccctggggctgctggggggcagaggcagtgcagaggtggagggtctgcacctgcacccatgcagaggaagagggtctcaggaggctacggtgtcttagccatcatcctacccctgttgaagggggtgtgcagggcaggaccacccggccttcctccctgacacaggtgggctgggcggtgctggctcatgcctgtaatcccagcactttgggaggccgaggctggtggatcacctgaggtcaggagttcaagaccagcctggccaacatagcgaaaccccgtctccactgaaagtacaaaaaaatcagctgggcttggtggcgggcgcctgtaatcccagttacttgggaggctgaggcaggagaatcgcttgaacctggaaggcggaggttgcggtgagcggagactgcgccactgcaactcacttgaacctggaggtggaggctgcagtgagctgagatcatgccactgctctccagcctggactccatctcaacaaaacaaaacaaaaagtgggtggggccttccagggggcttccgggtctgcctgggcccaggctccgggccttggcagagggcagctctgtcccagcggtgacatctgggctgctgctgctgctgctgctccaagcaggaggctgacaggttggctcagttgtcatggcaacatgaggggaactgacgctggatggactgggagggaactgtagcccctacacccggcattttccagcggggcccggctcgcttcatgttctcgtttgtaccaactatggggagcaggccctgctttgtggggacactgccccccaccttctactgtccacctcaggtttgctgaccagctcctggggtgaggagggggcattgacccccgctgtgcttggtgagcgaggacccccaggcagtcccctccagggcctcctggccacatgggcatcctccacccaggatgtcagggttctggcgagtttttacctcctacataggagagctgggtacctggcatccgtcctggccccagagtcggcacgggggctgggggagctgagtgctgtggcccagatcttggtcctcttgatgcagccccaggaaacaggcgcctcctgctgtgctgtcgcggggaggggtgtggggggcgagtgtggcaaccgcctccctcgccagtgagaccccataagtccctgatggacccccagctctgggtgggcaccagccccgcctttgacagcatttgctacctcctccctgaagccgccctgcagggggcgcagggacttagccggtcacccagcaggtgtggggacaggagccctcaccgcctgaccaggtgcccagcgccatccaggctgtgggtgagctcccaggcctctgccccacctcttcccgccccacccccccacccccccgcctgctgtggtgcacggagggttgagtggacgacctgaggctgcgggctgtgctctctgctgtaggtcacccgccatgtcgaccttgctggacattaagagcagcgtgctcaggcaggtgcaggtgtgcccgtccttccgccgcaggactgagcaggacccagggagtgccagcgccgacccgcaggagcctgccacgggggcctggtgggtgccgaggggcccagccccacacgcccacagggccatttcccgccagacttcacacatggccgcccctgccccagcacccctgggcgggaaggagggtctgcagttagctgtgccacactgtggatgtcacatggcccttcctgcccctggagtcgagggtcccaggcaggggccagcgtgctgagagctgtgagctgacccggctctttctccaggaaacccggggatggcgtggagttctttgcccacatgcgcctcatgctgaagaagggggaaggcagacagggcttgccgtgcctcgaggtaagggtgccacccccagggcccgataccacagcccagcatggggctccccctttgctcctccccgctccctcctgccctcgctcccacctcctggggcaccacctgtcttccctgctgcaggcctggggctctccctgccgtcagagccctcaccctgggccccacacacaggtccccctgcgcagtggctcgccagcacccccggagcctgtggatcccagcctcggcctgagagccctggccccagaggaggtaaaggtgggggtcggaaccaggggcagtcgtctgcagcggctgctccctcagccgtttagaggcacctactgtgtgcacagggtctggtgatggcagagagccagggtgagtgggtgggtgagtgaggagcggaggggctcctggtgcaaagaggcagaggtggggcctgggctcaggcagacaggaggtgctgctgagggctgggggcctgggacgcggcactgtggccgcccttcccccacctgcaggtggagatgctctacgaggaggccctgtacacggtgctttaccgcgcgggtaccatgggccctgaccaggtggacgacgaggaggccctgctcagctatctccagcaggtcagcccaccctgaccccgacccagaccctgacagcttccccacctcaaccccttccccacccactgacaccatcatcacccaaacagtatgcagaatctccccgatccgtgcacagcacacagagacggctgcaggtgcacacacacacacgcacacaggctgcaggtgcacacagacacacgcacacaggctgcaggtgcacacagacacacacacaggctgcaggtgcacacagacacacgcacacaggctgcaggtgcacacagacacacgcacacaggctgcaggtgcacacagacacgcacacaggctgcaggtgcacacagacacgcacacaggctgcaggtgcacacagacgcacgcacacacacaggctgcaggtgcatacagatgcacacgcacacaggctgcaggtgcacacagacacacgcacacaggctgtaggtgcacacagacacgcacacacacaggctgcaggtgcacacagacacaggcacacaggctgcaggtgcacacagatgcacaggcacacaggctgcaggtgcacacagacacacgcacacaggctgcaggtgcacacagacacacgcacacaggctgcaggtgcacacagacacacacacaggttgcaggtgcacacagacgcatacgcacacaggctgcaggtgcacacagacacacacacaggctgcaggtgcgcacagatgcccaggcacacaggctgcaggtgcacacagacacacgcacacaggctgcaggtgcacacagacacgcacacaggttgcaggtgcacacagatgcacgcacataggctgcaggtgtacacagacatgcacacaggttgcaggtgcacacagatgcacacgcacacaggctgcaggtgtacacagacacacgcacacaggctgcaggtgcacacagacacgcacacacacacacaggctgcaggtgcacacagacacacacaggctgcatgtctacacagacacacacacatacatgcacataggctgacagacgcacctcgaaggacgctcccggcttccacctgcatgtgctctgaactcccctgtcctcccgtggcccccattcccacgcatgcacttgcgtggccctcatcacacccaccatctctgggcatgcccaggccccttggcacaaaccctctgccgctgcctcccaggcttcccccaaccctgcacccgtgaggactgaggttgggtgtctgtgacgggctgagcccgaggtcccagcacagtggccagcctcgcttcaggggttgcctaggccctgccaacccagccaccctccacaggtgtttggcaccagccttgaggagcacactgaggccatcgagcgagtgaggaaggccaaggtgaggccgccactgcctgggcaggcactgaccagcacgtgcctgcgtggcgggggcacggggcaaggccctgtcacctccatgctaagtagggcatctggggggcagctcagcctcaccaggccccccacgcccctctgtccacaggcccccacgtatgccctgaaagtctctgtcatgcgtgccaagaaccttctggccaaggaccccaacggtgagtggggacccagcagacctcggctgcgccgaggcctggcggccatggagggccagagggcgtggggccagggctgagacgcctgccgtgcccaggcttcagcgacccatactgcatgctgggcatcctgcctgcctcggacgccacgcgggagccccgtgcacagaaggagcagcgcttcggcttccgcaagggcagcaagcgcggtggacccctgcctgccaagtgcatccaggtcaccgaggtgaagagcagcaccctgaaccccgtctggaaggagcacttcctcttgtgaggccctcgcccgtctgggtgcgggaggggggctctgcctggggtccagagctgggctgtgcctggagggtggtggctcgcagcagctcccggtctctttggggccgtgtgcccactttccagaggagaaaactgaggcccagagatgggcgttgtgccaccggggccacacggctggcaacacctggcgggatcaggatttggacctgaacagcctgtctggagagcgggtgcttgtggcctggtccctgctgcttctggactccctgaccccggcctgggcagagcctcgccggggaccctcatgggcatctctgttctccttgtagcgagattgaggatgtgagcacggaccagctgcacctggacatctggtacaggcccctgcgccatgcagggcggcggggatgcacaccttttgcgggggagcaggacggactcagggcccggctgcgggctccagacaagccaggtcctcccctgtccccaccagggatcatgacgacgatgtatccctggtagaagcgtgcaggaagctgaatgaagtcatcggcctgaagggcatgggcaggtatgactgctgggacctttctacccatcacccgtgccctcagcttggtcatggggcccggcgggcagtggctccccaggagccatcagcgtgatgctcaccacctgtggtggccaggtacttcaaacagatcgtcaagtcagcccgcgcaaacgggacagcaggacccaccgaggaccacaccgatgacttcctggggtgcctcaacatacctgtccgggtgagtgggtgtggggtggggctggtgacacttagagaagggcctggggagtgtcccagccttcaaggggcagggcactgcctggagaagctcctgcttttgggcctccactgagtgcgcttgtcaccctcattccccggagaagggaagcccaggccatgtggtctctccagggtctcacagagtcagtgtggaaggggagggggaggagtctggtgggcctgacccccatgctacccccaggaggtgcctgtggctggcgtcgaccgctggttcaagctggagccacgctccagtgcctcgcgtgtgcagggacactgccacctggttctcaagctgatcactacgcaggtggggaaagtgggcgtccccgtcctccacccccgtccttggggcgggggcagagctggtgactgggtgggctctgcgttgcagagggatacggccatgagccagcgcgggcgatccggcttcctgtcccacctgctgctgctcagccatctgctgcggttggagcactcagcagaggaggtagtgggtgcgcctaggattggaacagcccggcagggggcctgaggaggggatgtggggctgtcccgcctccacccacgacagacctcctcccccagcccaactccagcagctggcgaggagagctcagcacaccagccgccaccatcctctgcctgcacggagcccagagcaacctgtcacccttgcagctggccgtgctgtgagtgggtggagctacgagtgggcggggaatgtgggcgggcgtgagcgagtggggcatcgggggccatgcggtgagtggatgtcgtggctgggagggtggggcagggaaaggggcggtgctgagtaggtggggctgcgctgattgggcgggaagggaagggggcggtgctacgggtaggtgaggcagcgaaaggggcggtgccatgagtaggtacggcagcgctaattggagggcagggaaaggggcagtgctatgagtaggcgcggcagtgctgattgggtggggcagagaaaggggtagtgctgtaggcggtgctgagtgggcatggcaggggcggggttcataccctttgaccatgggccgggccccacaggcactggcaggtcagcagccgccaccatcaaacctgcacgctggactacagctacctgctggggctgctggaggacatgcaggcacactgggaagaggctccttcactgccccaggagcaggtgggtgcagccgggaccttcttgccagccatggcgaagggagtgctggggactggggtcgctcagtgccggtcggcgaggggcagagtcctgcccgcgtgggggtcaaagacaaatggacaaactgtatgacgtgggcgagagccagcgctctgcagaaaggagacgggagactcgtgggagtcacctcggggggcggccaggacttaggacagggctggggaaagccgccaaggtggcgcccatctgagcgacaggagggagcggccctggcaggacggacgtgggaactgcaggggcacaggccctacgtgagctgcgtgggtggaaaccgaggctgggacaggccgtctgggacaggccgactgggcctgtggagggtgctgctcagagcagagctgggctgaccatgcccggggaagggtgttgcgggccaaggcagggaggatgtttctcctcatcagtggccggtcggggctgctggcactgaagggccctgtccccacaggaggagagcctggctgatagcctttccgccttctctgagttcgggctgcagctgctgcgccagctccgagactacttccctgccaccaacagcaccgctgtccaccgcctggagctgctgctgaagtgggtgcagcgccgcgtgtcagcgtgggtgggggcggctggcagggcaggccccacgtcagcgtgctgcccccacccaggtgtctgggcaagctgcagctcttccaaccctcctttgagatctgccccttcgagtcggagctgaacatggacattgctgcggccctgaaggtgtgttccaaagcccagtggaggccggctgctaagccctcccttccccttccctgccttcccgtcccctgcacctctgcaccacggtctcttgcccacctcttcctcttccctgcaatccaccatgctgtcttggtggtgtctgttgcagagaggcaaccgtgagtggtacgacaggatcctgaatgacaagagtccccgagagcaggtgcagttgtgggggaccctggccatgaggggtacgggcagccgagaggtgggtacgagctaggcttccttgcagccaggaccacagcgcctgcctgggctggttgtgctggctgacgccgtctatgatgaccttcagttctgctacagtgtgtacgccagcctcttccacaggtgggcctggcccctcagtgctgtcctggggctggggtcggagccaacagggcctgcaggtggggcgcaggtggatgaggtgtgtcccttgctctgcagcatcctcaatgtggacgtcttcaccctgaccttccggcagctggagcgtctggtgaggagggtccctgaccccgggtgcctgccaggcatggggaagtgggcagatgcccttggctaggacggtcctgggattccttgctgctggaggcctgatcctgctgtcccggacaggtggctgaggaggcgtgggtgctgacggaggagctgagccccaagatgaccctggaggtggcctcggggctctttgagctctacctgaccctggctgacctccagcgcttctgggatagcatccctggccggtgggtgccccgtccctatctcttgcagacagactttgggaccagggccccaggacctgtgagggggacggtcctggagctgtgagcctggaacgtgctggttaaggtgtctgagtcagggccgagccctcacaaccctcccaccacagggacagccgctctctggccctggctggcatccacgcccccttcctgcctgctgtgaagctctggttccaagtgctgagggaccaggccaagtggaggcttcagggagccgtggacatggacacggtgacagctgccctggcctgaggacactggggctggtccaggcccccagcctcccccgcctccccagcaaccccagcctccccagcaacccccagcctcctccgcctccccagcctccccctcaaccccagcctccccggcctcctccgcaaccccagcctccccagcaaccccagcctccccagcaaccccagcctcctcagcaaccccagcctcccccgcaaccccagcctcctcagcaaccccagcctcccccacaaccccagcctccccggcctcctccgcaaccccagcctcccccacaacccccgcctcccccacctccccagcctcccctgcctccccagcaaacccagcctcccctgcctccctagctggagcccgtggacgcctcctccaggcacagcagctccgcagccactgctggtctctgcctcagccacatccaggagttgtgggtgcgcctggcgtggcctgaccctgcccaggctcaggggctgggcacccagcttggccaggtgtgtgggtgggccctgggggtgaggggaacgggtgggagaggagatggggcaggggagggctccatggctccccaccgccatcccctcctcaggacgtgtgtgaggccaccctcttctatacggagctgcttcggaagaaggtggacactcagccaggggcggccggtgaagcagtgagcgaggcggtgagtgaccagctggggaggggagccggcaggaggtggggggccatcaccaggccccggacccatcgttgcctggccacacctcctccagctctgcgtggtcctcaacaatgtggagctcgtgcgcaaggctgctgggcaggccttgaagggcctggcatggccagagggggccacggggcccgagggggtgctcccccgccctctgctcagctgcacacaggccctggacgatgatctgcaacgggaggcccacacggtgacagcgcacctgacctctaaggtgggtggggcctggagaccaaggcgtggaggcagtccctggtgccccctgcccgtgctgagcactgctcctgccctcagatggtgggcgacatccgcaagtatgtacagcacatcagtctctcgcctgactccatccagaacgatgaggtgagtgccggggcgaggggccgtggaggactgtgtgtactgggggtagggcagaggtgcggggtaagcctggcctgaccacccctgcccgcaggccgtggccccgctcatgaagtacctggatgagaagctggccctgctgaacgcctcgctggtgaaggggaacctgagcaggtgcgggcgggtggggtgggatgggctgggctggcccgtggtcactgaggccgccctgcagggtgccggaggccctgtgggagctactcctccaggccattctgcaggtggggccagcggggcaggtggggctggcccgtggtcactgatgctgccctgcagggtgctggaggccctgtgggagctactcctccaggccattctgcaggcgctgggtgcaaaccgtgacgtctctgctgatttctacagccgcttccatttcacgctggaggtagagctctgtgaaggagtcctccccgccggcccccgcctcagggctgctctgatccctccctttgtccccactggcctgcctggtctcctgtggctggagagcagccactccagggctgtccacagcgcctcctcccgttaatgccgaggtgtggcctctggtgatggcctttccccacgcttcctgggagcttccgatgcccgccagggtgtgagaaagggctctgcccttggctgctgagcccccagtgctgcctgggcccctttgtgctgggggtctctacctgtccccagcacaagccaggctccctgacacctctgccttctttccctgccccaggccctggtcagttttttccacgcagagggtcagggtttgcccctggagagcctgagggatggaagctacaaggtgaggtgggaccctctgtggggcgggggtgtggatgaggggactgagttcaaactgcagccccacacgggctgtgtccttgagcatgaggtggccccacgggcagtcaggtctccaagtgccagcgctgaccaccagcacccaggggcccctcctgatgcgcttccccctgcagaggctgaaggaggagctgcggctgcacaaatgttccacccgcgagtgcatcgagcagttctacctggacaagctcaaacaggtagggaggcgccagggacagggtgctgcctccgaggctcccagagcccagctgcgctcacccgcctttccgcagaggaccctggagcagaaccggtttggacgcctgagcgtccgttgccattacgaggcggctgagcagcggctggccgtggaggtgctgcacgccgcggacctgctccccctggacgccaacggtgagttgcagcggggacgggtcgggtggtggtgggatgggggcaggggggctcacgactgtgctcctgcaggcttaagtgacccctttgtgatcgtggagctgggcccaccgcatctctttccactggtccgcagccagaggacccaggtgaagacccggacgctgcaccctgtatacgacgaactcttctacttgtgagtgtcctaagccccagccccagccccaggctccaggctgccggagcgagactcccgactggcctctgtccgcagttccgtgcctgccgaggcgtgccgccgccgcgcggcctgtgtgttgttcaccgtcatggaccacgactggctgtccaccaacgacttcgctggggaggcggccctcggcctaggtggcgtcactggtgtcgcccggccccaggtgggcgggggtgcaagggctgggcagcctgtcaccctgcacctgtgccggcccagagcccagggtgagtgagcatctgggtggaggcaggggcagcgggcctgaccccggcccccacacacctgcccgcgctgcttgcagtgagatctgcgctgaggaggctggaaggccgcaccagcaaggaggcgcaggagttcgtgaagaaactcaaggagctggagaagtgcatggaggcggacccctgagtccatcagctgccagccccggccctggcccccaccccaagttccctgaagcatcctccagctcactgtggccagctttgtgcaaccagggcccacggcgcccctcctgtgctgtgacgtgtgtgtcgtggctggccccgcggcgcctaccgccctggccgtgtctgtctggtgtgtgctgtgaacccctgcacccaaccccacatctgggtggccaacttggcaggacttggccagcagctgcccaggacacagtgcaggccagagcgggcttgaccacctggtgggcctccctgcccgcttccttgggctccccggccctgggtgggcggtgggcagctggtctccagggactcagtgagtggctgtgctctctgcacaacgggcaatgtgcagacgcatttttggtaatcacagctggggagtgaaaagggtgccactggcaccactgggtggatggtccagagcctccacccacagaggggatgcaaagggcaggtgagtcaagaaccgcataggtctccagtccccacggggctcccaggccggggaaaggttcccctgaggtcactctgaggccagggacgtcacccaaggctggtggtcagtgtgaagggctccgtgccaactggtcagctgtccttcacgcacatatccgtggccacctgagacctgctccacgacccttccaggcagagccgagagttcgccccaacccttccccaggcccagtgtgaaaaacagactcacaaggggcttcttggcctgcagcttcatttgcgagagcgccgaggcaggacacagagcacagctgtgctggaagtgtggggagaacccggacagctcagtcctgccagcagccgcaaagagccgaggctgccaggcccatttatgtccctcatgtctctagattttctcgtcacccagcctcaaaaatatatgtgtctgcaaccctcagtctctctgccgtttcttgattcctttccaaacctcagccggggccctggcctcagccccccgtccctgcgtctgctcctcttcacagcagctgctgctggctcctccgcgctcggcgccaggccctgggtcctcagacgcatcactgtcatcagtgtccgagggcagcctgggagaggagggggagcacgttcccaaatgacgtcctccctggctagctggagtcaacgtcatccacaaaaagcccccagccgaggacagattcctcttcctccctgtgctctccacacccaaacctctccagtgggacatgcactgcaggttttctagtcagaagaccatcggggtgttgtttacaacaccacccccaggcagcaggaagggctcaggccagaggtagaagagagccctgggtctgagtgatcatgaaattaagcccaaggacctaccgggtgctggccacaggcctgttggggtttccaaactctctccctgaatccacatcgaagggatctgagccgagagaggaaagtggcctctaagtgagctcagagcaggaccaggcctcccaccccccaggctttggagggccttggttcccaccccgccaaggctcaccgatctcctcctcctgggggctctccttggcattggccaagaactcctgctccgcctgcagcacctcctccgggctgccgcaggccgcgtacttgtccaggagctggcggttcaggtccaagaagcccttgccccatttaaacttccgcagcaaaatgacagcaaggtctggaaagcctgacggtgtgagaaacaggaaacccaaagaagtcgacggtcccctcaagtgcctcctgatcaccctgctgccggatggcggcgctctcccaagtaaccgcagggtccccagcaaacatcagctcacagtcctgagaacccgtgctgcccccagcctgcacctcccttgctacagtccacacctctgcaaccgaccaagtccccctcctttgctgcgaaacaaacattcccaccaagactgccccacgcctcatcagggcctctttagatcagcaattcagtaaggaggacgtggaggataaggaggattctctgggcagtggtgccctgataatgaatggcaccccacacaccggccctgatagaacagcgatcccattctgcctctactgcaactccacaggccgaaaacctgaagtgacagttggggctggaaccgtggtctgtctgaactgttcccctgtccgtcccttatgctcctcctggggtctgtcggcaggaaacatgatgctgggagcatagcaggaacagcaggccgccgggtcacactgctgtggggcccctggactcacctacgatgcagaaggtggcagcaaacgcttccacgcaggaaagtctgtagggccggccatagttcacggggttggcggccaccaggtagggcaacaggcgcaagtggctccctcgcatcttcccaaacggtgtctcgtccagcctggcccaggagcagtcgatgacggcgaccccagactgcgccaccagctgtctgccagggacagcatgggataagttcaggagccagcactacccaaggtggagcatgccccggagaatggcctagctaagggattcagggacatcattccgccatccccaaacaccctcaaacggacttcacgagcaagcttttgaggcagctgcttggggcacctggagcatgtgtttattcactcaacaaacgtttattaccccaaaacctcctttatcccaggctctgcgctacgcaacggaggcaaactagacagacgttccctgcccacgtgggtgtggatgtgggtgcgacatacaagtgccactaaaccatccctgaagggaaccacgcgctggaaatgaagacgacctcgggcaggcctcccgcgcctacctgtctgcgggggacgcgtactgcttgcccacggggctcagcaccagaccgccgaatctgtggcccaggcgcaggcagcgcaccagccccaggcgggccagcttgcggcccgtgcagcgccgggggtcgcagtggcccaactcccacatggccagcgtgcagggcagcgccgccgggcccgggccgccctcgccgtcagccgcccctggctccacggaagctgcacgagagagagaagggcactcggcctcagcgtgggacccccgggcaggccctcaaaccctgacccgctctccccatcacgcctcgctcaccctgcagcgcggcgccgacctcctcggcgaaggcctccagggagcgcgtcgggaggtgccgagggcggccgccttccgcccccggcccgcgcgctgccctcctgcggcccatggcgcggacctggggtgccggggactccccaccccacggccgcgcccctcggcctcccaatgggctgtgcggctgccagcgcttgcgccggacgccacttccggtccccgtggtcacgtgaccgtcacttcacgtgaccgcgcggcggccgctgcggcgcgatggcggcggggctggcgcggctcctgttgctcctcgggctctcggccggcggtgagtggcccggccgtccgcgtccccaggccccgcgcgcgctctgcaccccggcctccccgctcacggtctcgctccccgtagggcccgcgccggcaggtgcagcgaagatgaaggtggtggaggagcccaacgcgtttgggtgagcagcctcgcgggctggcggctcgagcgggggacggcccgggcccgttccccgctgaccttgccgcttcccgtagggtgaacaacccgttcttgcctcaggccagtcgcctccaggccaagagggatccttcacccgtgtctggtgagtgaggagcgctgttggccggcgcgggggtggccgcggggagcagcaacagtggaggatggatgagtgaccctgagagttacggcccggagtctaaagcattggtttgtcagccttctaaaattaatgtggaagtacgagagaatataccagaacgttcttgtagcgaattaaattatctccagacttagtccttcctttcctttccgagaggtaactgctgttaatagtctggtgactttgcagacgcgtttctgtgcttttacgtgcgtgtctatgcaactggaaaacgtatagtttttgtaatctaatcgcttttttaaaaaatcatgctgcgcgagtatttggctgatttgtcgttgtcactcgctgtatctctcgggacgcttcatggtgatgatataggtcttcgttctttctcttggtgctcagcacgcgataggatggcactgcactccccgattgtatttaggttcttgccatttttttggccattagaaatagttctgtggagaacatgctgtgtaggtgctttggtttgccctcaggtgagcggtttcccagtgcaggattccagaagtggaattggtgggtcatgagtatatatatcatttaaacgctgaatgagtattaaatataatagacaaaaatccaagaatttttttttttttttttttgagacaagagtctcgctctgtcgcccaggctggagtgcagtggcacgatctcggctcactgcaacctccgcctcccaggttcaagcgatcctcatgcctcagcctcccgagtagctgggatcacaggcatgcatcaccatcacgcccagcaaatttttgtatttttagtagagatagggtttatttcaccgtattgaccaggcgggtctcgaactcctgacctcaagtgatccgcccatctcggcctccaaaagtgctgggattataggcgtgagccaccgcgcccagctttagtaattttattatcgaatacctttatgtgttagggtctgtgctggggttgcagaaacgaacacagatataccaatgcttgtcctcatggagctgacagtctaatggggagggatggaggaacaggcactgagcctgtagaattagataaaatacatagaatggccaatgatgaaaaatgccatgtagatgtgtgaagcagaggagggagtgtgtgtatgtgtgttagagtattgtttgggggatcgctttttttattttttagagacggggtcttgctctctcactcgggctggagtgcagtagtgcaatcatagctcactgcatccttgaactcctgggctgaagcaatccaccaacctcagcttcctgagtagctgggactacaggtgcacgccaccatgcccggcaaatgttaagtgttcttgtagagatggaagtctcactatgttgcccaggctggccccaaactcccagcgtcaagcaatcctcctgtcttggtggggattgctttaaatagggtgctgaggaaaagtctaagaagtgacatttcagccaaaagctaaaggagaaaagggagtaaagctgtggctatttggggaaagggatccaggaaagggagtggccaaaggccttgagcaggacagccagggccagaggggattcagctggggtagccacagatcagagataaaggggatcagatggtgccgcaccgtgttggtaggtttggtggactttgactccaaacacatctttttaagttttttgttgtgtggctgggcgtgatggcgcacctggggttaggctcacttccctgcggctcagaagccatgggatggtggcttcccagggcctgaagtgctggagtcgggccaccacctatcccaagaagctggggaggaggcaggccactgtgtaacagcgcagttcccgaccgaaccccttccagtaggagagaggcagccttggctaatgggaccacgtctttttggaggtgagagtcctggaaagaaaacagaatatgctggtcgtggtggtgcgtgcatgttgctcgggagcctgaggtgtgacgatcgcttgaggtcaggagttcgagaccagcctggccaacatggtgaaaccccgtctctactaaaaaaaaccacaaaaacaaaagttagccgggcatagtggtatcctcctgtaatcccagctgcttgggaggccgaggctggagaattgcttgaatccaggaggcggaggctgcggtgagctgagatcatgcccctgcactccagcctgggtgacagagcaagacttcgtctcaaaaaaaaaaaaaaaaaaaaaaaacccataaaaatttagaaaagaatacctcttctagttttcccacccccatccccacatcagggccagtcaggttcacatgtgcatttggttcattatgtgtctttagtgttttaatttggaacagttctcttcccacctttggatgtaagtggcacatttgtttcccagtttcctgatctttttgttttgttgatggtgtttttctccagaccattttttcctgcgcctgtggggtcaggcgtggatagtcccccgcgtctgtagggctgggcgtggatactcccccgcgtctgtagggccgggcgtggattgtctcccacgtctgtggggtggggccgggcgtggaaagtcccccgcgtctgtggggtggggcctggatactcccccgcatctgcggggtggggcgtggatcgtctcccgcatgtgcggggtcgggtgtggatggtctgccgcgtctgcggggtcgggtgtggatggtctcccgcgtctgcggggtcgggtgtggatggtctcccgcctctgcggggtcgggtgtggatggtctcccgcgtctgtggggtcgggtgtggatcgtctcccgcgtctgtggggtcgggtgtggatcctctcctgcgtctgtggggtcgggtgtggatattctacttgttttttctgtctttgatgtagcgctccttcaacatcaacgtgtgaggcggattgtgtgcagtgggtaggagagtgtggtctgtggaatgagctgagcagaaagataatcaggctgagaaactcaggggcgtggtgattgtggcctagggctcagaaggtccccgcagaagagtgtctgccaaggagaaggaaggggtgggcttgccagggccgaggctggtggggctgggctgggcagtgggcagctccatggcctccaccagatgagtcgggagcctcgtcccttgacagcaggaggagcagaggttggtaggtctgtgatttggagacagtgtcccgctgtggcatggagagggaatggggcacggtcagtgtggtggtcctgccagcgcccagcagagtccagcatagcccagcacactgtccaggagggagctctgcctcggtggcggtgggagggagcggccagctgggcaggactctggggccttggcagggtcctggatctggggagggggcgggggaggagtagagtggccccgggatcctggctagagaagccatggcaagtgcggctcccagcatgtgagttgcaggggtcaggctgggagcttgctttcggctggggccgggctgggtctgccaggcagtccctggtgcttggagagggtcacggtgggcacaggactcaccagggggtgcgtgaggccggagggtgagggtgaggtccccggtgaagttcttggtggcgtgtgggggttgtggaggttgggagggtactgggaccaggtgtggggccgggccagggagcagcgtgggagggaggaccacccttgagaagggattgtcaccaagagacgcagccctagggggagtgctgcccaccgaagcctgaccggggcagaggggaaacggagtccaggctcagccaagctgatgggccttgagctgtggcctcgggtgcagggacagctggcagtaaggccacctgggcccgcgggagccaagggcaccggcgagtgtcctggccatgcaggccgtgaacacggagcctgcaggggacgtgcgggtgaagcaggagcagaggagaagcctcccggcaggggtggtggcatttgggggtggtggtatttgggggtagagactcccgctcaggtatggctgtgtctggcccaagaggcgtggaggggccagcccccacctgtctcaggaggacaggctgctgaaacctgagggccaggcggaggggaggggccgtggccggggttctggtgtgggccagcagcagaggacctggggtactgggcactgccagttgggggcttgtggtaccatcccctgcgggaggctgcacctcagccagtaggagtccactgaagctgtggacagcacccaggcagaggcctcctcccagctggggagccaggccaggccccgctgccctcacacagtcctgtttagacggatggccctgcaggtgcagagctaagcccggcgcccacgcccctcctggggtgtctccaagcagtgtctgcctggggcaagcctgggaacgtgagttcccaggacacaggagcttgaccagccaccctcccagcgggagtgtgagagcgggaatctgcgggcgggggtctgcgggcgggagtgtgcgggcgagagtgtgcgggcgggggtctgcgggcgggagtgtgcgggtggcggtctgcgggcaggagtctgagtggcagatgggtgagggacacatcagggtctctgggatggctccaaaggcttccctatgtgcctgttgggcagggccctagggccagactggagggaccaagcccaagcaccccaggctgtgggacagcaggtgtgacagggctgaggtagagcccccagcggtcaggctgggaggacagtgggtgagcctgggagtaggggccacccagatggtatttgacaaggaaggaccaggacctttctatatccacagcccctgtcccctgagcagggcagcctggccatcttggtctccatctgtcttgggaggggacagccccaggggtgagtggaatgtcagggaacagaaggccaggccgggccagcaccccagaggtctcagtggcattagcctctgccctccgggggcgggggccaggctgagggtggccccagtgtggcatctggctcctgggctggccagattcccagggaacagtcacccagcacctcgtgtgtatttcgactttattattattattattattattattagagccgagtgtcgctgtgtcgcccaggctggagtgctgtgtcgcccaggctggagtgcagtggcgcgatctcggctcagtgccagctccgcctcccgggttcacgccattctcctgcctcagcctcctgagtagctgggactacaggcgcccgccaccatgcccggctaattttttgtgtttttaacagagacggggtttcaccgtgttagccaggacggtctcgatctcctgacctcgtgatctgcccgcctcggcctcccacagtgctgggatgacagtcatgagccaccgcgcctggcctcgactttattattaatacggtaaaactacagagagtttgaaacatcagtctggggcgtcctcctctaaccagtgcacttgctgctgcagcgcaggcccttcaggggacggggatgggggccagtcccctccctgcctccacggcccaagccccagccttccccacagtcctggaggatggcccgtggctgacagtctcggtgggcgctgtgttaaggtggcgtccccagccaggtcccgactccccagcttaaagaacagggtggctgctgtggcttgactcacgagttgccactggccttgatgtgtacacgcccttgctctagcactgcctggggctggcctggctggagggtagggacgctgcacgccttccctcttcccccaggcgctggtgacactgccctgcaaccgtcagcaccatcatctccacaacttgctctgtccccttcaaaccctgactcccagctgccccgctcccccagccccggcacccaccattctgctgtctctgaacttggctattcatagacgtcacataggtgactcataactgtgtgcgaccttttgtgtctgaccaactcctcttggcacagtgtccccaaggtcccccaggctggaggctctcagtgcttcctttttatatggctgaggactgttccactgcaaggacagaccacgttgttgtttacccgtcatctgtccatggacactggagtggcttccaccctctgcctgggaataggctgctgtgaacttgggtgtaaatatttgagtctctgcttccaatttctgagagtagctttgctggatcttattagaattccatgtttagctttttgaggaaccaccaaactgtttgcatagtggctgcaccgtttcccattccagccagcattgcacgaggggtccagtttctccacattttcaccaacactgttttttgctaacagccatcctagtgggtgtgaagcggtaccttagggttgttttaatttgcatttccctaatgatcagtcatgtgggatcttttcagttgcttatggtgtttgtccttttttttttttgagatggagtctcactctgtggcccagactggagtacagtggcgccatcttggctcactgcaagctctgcctcccaggctcaggcgattctcctgcctcagcctctcaagtagctgggattacaggcgtgagccaccacacctggctcatttttatttagtagagacggggtttcaccatgttggccaggctggtctcaaactcctgacctcatgatccgcctgcctcggcctcccgaagtgctgggattcccggcttctctgcccatttttaacttgagtttattgttgggctgtagttctgtgttctggatgtgagtctcttacatacgtggtttgccagtgtttattttctcccattctgtgggttgtcttttcactcccttgatgggtttgtttttttttccatatagagtctaactctgtagcccaggctggagtgcagtagcacgatcttggctcacggcaacctctccacctcccaggctcaaggaattctcctgcctcagcctcccgagtagctgggactgcaggcatccgccatcacgcctggctaatttttttgtactttattagagatcaggtttcaccatgttggccaggatggtctcgatctcctgacttcgtgatccgtctgcctcggcctcccaaagcactgggattacaggcgtgagaccctgcgcccggcctctatgccagtctttatgccagtatcacactgttttgatcataacagctttgaagtaatttttggaatcagtatgtacgagtctctcaactctgtctttaagatttgttcgatgatttggggtcctctgagactccctatgaactttaggatgtgtttttctatttctgcaaaaactgtcattggggttttgatggggttgcattgaatctgcagatcactctgggtggtattgtcatcttaaaaatattgaatccatcaggctgggcgctggggctcacgcctgtaatcccagtactttaggaggctgaggcaggtggatcacctgaggtcaggagtttgagaccatcctggccaacatggagaaaccccgtctctacaaaaatacaaaaattagccagccgtgatggcaggcaccggtaatcctagctactggggagactgaggcgggaaaatcggttgaacccagtggtggagattgtagtgagccaagatcgtgccactgcaatccagcctggcgacaatgctagactccgtctcaaaaaaaaaaaagtggcgtatggatgtctcatgtcagctttatgtagtgggtggcactgtttgtgcgtggataattgtggtgtcttggcacattgacccttttatcaatgtataacatctttgtctcttaaaatagtttcagccacttcagctctcttttgcctgtttatatgaatacttttccataccttcactttcaacctgtttatgtctttaaatctgaagttagtctttcaggccgggcatagtggctcacacctgtaatcccagcacttgggaggccaaggtggacggatcacttcaggtccacagttgcagaccagcctggccaacacagtgaaaccccgcctctgctgaagatacaaaaattagctgggcgtggtggcgggcacgtgggatcccagctactcgggaggctgaggcaggggaatcacttgaacccaggaggcggaggttgcagtgagccgagatcgcgccactgcatttcaccttgggtgacagagtgagactgtctccaaaagaaaaaaaagaatctaatgcctgatgagctgaggtggaacagtttcatccccaaaccacccatccccacccccggctggtagaaaaactgccttccatgaaaccagtccctggtgccaaaaagattggggaccactggtttaagtcctgtagctttacagaccatagctagaaaggcaactggtattaattcaccctgcacgaggacctccgtctgcctccgctgagctgctgtctgctcacttccccgggtggcacaccggcctgcatgtaaccaactcctgaagcttttatctgggaatgtcctcttttttggggggtggggaagacagggtcttgctctgtcacgcaggctggagtgcagtggtacgatctcagctcactgcactctccacctcctgggttcaagagattctcctgcctcagcctcctgagtagctgggattacaggtgcgcgccactacactcagctcattttttctgtgtgctttttgtgtagacacggggttctcacagtgttgcccaggctggtgtcatactcctggcctcaagcaatcttcccgccttggcctcccaaagtgctgggattacaggcatgagccacgatagcaagccttaactctaatttttgaagggctatttttagaattctcggttttgtcagtttcttccattgaatggtacctgttttcctgtttctttgaacgtcttgtgctttttgttgaaaactggtccttggccgggcgcggtggctcaaacctgtaatcccagcactttgggaggccgaggtgggtggatcacgaggtcaggagatcgagaccatcctggctaacgcggtgaaaccccgtctctactaaaaatacaaaaaattagccgggcatggtggcgggcgcctgtagtcccagctacttgggaggctgaggcaggagaatggcgtgaacatgggaggcggagcttgaagtgagccaagatcatgccactgcactccagcctgggtgacagagtgagactccatctcaaaaaaaaagaaaactggtcctttgaaaacagactctgccagtctttgcagacaggttctgtgcttggaccctggggatcagtgtgaggtctcttccaggacccgtgcatctcttccgactctcgggcaagtgcttcagcctggtggagtccacgtgagtgcagggtgggtgcgagggtgggctggggcgcagcctgcggacccccctcatgccatctgtgtccccaggtacaagtatgagttctgcccgttccacaacgtgacccagcacgagcagaccttccgctggaacgcctacagtgggatcctcgggtgagtggggccggggcagggatcccaaagcagcagcgcagctccccacccggcctcacgtgccgtgcccgtgtctccccagcatctggcacgagtgggagatcgccaacaacaccttcacgggcatgtggatgagggacggtgacgcctgccgttcccggagccggcagagcaaggtggggcctcagacgggagcccgggaagaggggccccagtctccccaacccacctacccacgttccctccccaggtggagctggcgtgtggaaaaagcaaccggctggcccatgtgtccgagccgagcacctgcgtctacgcgctgacgttcgagacccccctcgtctgccacccccacgccttgctaggtaggggtgcgggacgcagttgagcccagtggggtcagccgcgcacgcagccctgctggaggccctgtagtgctgggggccagggttgggacatggggtgcagctgagcctggcttctcttgggtcctcagtgtacccaaccctgccagaggccctgcagcggcagtgggaccaggtagagcaggacctggccgatgagctgatcaccccccaggtaagcgtgcgctcggggtggcccctggtgggcctggctgggagctgggtgctgcccctgcatcctccaccttcagggccatgagaagttgctgaggacactttttgaggatgctggctacttaaagaccccagaagaaaatgaacccacccagctggagggaggtcctgacagcttggggtttgagaccctggaaaactgcaggaaggtaccgtattggggggaggtggtggcacgcagtagccctccagcacctgggctttcccttgaactctttttgtggttggtaggctcataaagaactctcaaaggagatcaaaaggctgaaaggtttgctcacccagcacggcatcccctacacgaggcccacaggtgagtcacctgtggggagagggccaggctcaccatcacactcgccacctgtgggtccaggtgaggactggccacctggtgttttggcagaaacttccaacttggagcacttgggccacgagacgcccagagccaagtctccagagcagctgcggggtgacccaggactgcgtgggagtttgtgaccttgtggtgggagagcagaggtggacgcggccgagagccctacagagaagctggctggtaggacccgcagggaccagctgaccaggcttgtgctcagagaagcagacaaaacaaagattcaaggttttaattaattcccatactgataaaaataactccatgaattctgtaaaccattgcataaatgctatagtgtaaaaaaatttaaacaagtgttaactttaaacagttcgctacaagtaaatgattataaatactaccttctgggttaagaaaattccattcaaataacattctcatgtaaatactcaaacatacagattgaggcttccagaaattaatccacttgaggcgtccacgcggaacaaggtctgctgaccacagttacacacgtcgtgacaccactgtatcacggcgaatgtcgaacactagagttacagacgacaggcaacaagaacatgcagagccggccgccagccagctcctacgccccgtgcccgccatggccacaggggggagctgctgggcgcgcctccacctcagcctccacggggcaccttccagccactgctgtgcctcagccacctaggagccatccctgaggccatggccaccaagacaggtgagggaggccccagggcacacagccccaggatcttgcccccatttccaaccctggcagcgctgccagccattctttggtcttcccactgctcacctgtcctcagagccagtactgatctaggggagcaggtgccacctccatctcccagctgtccccccacccctggggctccccagctctaccaccccggaagctgagccctggagacaccctgaggggcgggcgccacctccttcccagaaagagcagagcagctggcagactagagctcagctgctcctgaccactgacaaccgggagatgtctcggcagacaccacttatcccagaaaagaccagaggctccaaatgggaaccaagtgcataaatacaaataaaaacagagtttacacatcgatgacagtagcacaaaatatacgtaatttctagatactgagctaataaatcactgtagttaaaaacaaaatagattcactgaaaccaagtcgatagttaccttggagtagtggactagctgcagaatgtcacggacccacttcctttaaaacaacagaatgttgctatcagtttgtcttacgttaaaacagttcttaaaccacctactatattaaatacattctaatttggtcactgatgataaagatttttacctagacgttttgcacttaaaaaatgctattaaaagtctttggcaaagccacggtcggggagaaacacgtcggtgccgcttcccactcgtgtgccctcaccacccacgtggcccgcggagagccaggtcgccgtaaaccacagatgcctgttcctgctcaactcctagggacagtcccagccctgagcagaagggtcaggtcatcgcggggatgcgttctcagtcacgtttctgctgcggccgaaaccacactgtcgctccaggggcagacgggacaggggaccacagcagagccgggacctggggggggtcgcttcccccgtgtgctccaggccactgagaggctgtggccgggtcctctcctgtgacctcactggccacatggatgagctccgatgataactggcagcgcgggtcaggtgactgccacatacacccggcaccccggaccctggcgcacacacggcccgagcatctcccacacgagcaggaccagggtggtcacgggtgctggggaggcaaccactctcccagttcactgcctgacccctgccaggacgcccagcgacatctccaaggagctacaaagcaccattgcctaggacagctctgagtaatcagaaaacagctttttttttttttttttgagccagagtctcgctctgtcacccgggctggagtgcggtggcgcgatctcggctcactgcaacctctgcctcccaggttcaagcgattctcctgcctcagcctcccgagtagctgggactacaggtgcccgctctcacgccaggccaatttttgtatttttactaagacggggttgcaccatgttggccaggctggtcttgaactcctgaccttaggtgatccacccgcctcccaaagtgctgggatcacaggcatgagccaccgcacccagccagaagacagctgcttaaaaaagtaattctaaaagctctgacgtcggatcccctctgctgcgttttttgttagtatcaaactgtctttcagagggttaagggagaaaacaaaacccatgatgctccttcacttgctctccgaggcgcaggcgcacagaaacagcggccacggaccacagaaatgcaggacggagctctcctgctcccacgccacgaggctggaacatcagccccagtgcctggtgcacacacaggctcaggaaaactagctccttccatcaagttaaaaacatctcaatccataatcactctgcttataaatactatttgcacttaattggaaaatgtaaaaatcctaggtcttactactagataaacactcatttcataagtactagtttcagaatatgaaatctaatatatttagcatactatgaattgacaataaactgatatgaaatatttaagttcacagtacacatataaagcttcatattatcgcatattaaaaaaaaacagaaagcagcttagagcaatttataacctctaactaaattcctcggtttacaaagtgctttgaaagaccccagctcccagtggcctagtcacagtagtgtcaggaccacaaactgctgtgatcacagcgccgcgtggatcccggaggcaccaggcccctcagggacaggcaggcgggttctgtgggtttgcatttaaggtttttgaggaaaataccttgaaaccgtcggtaggactagataggtgacaacgtgtgacaggaaaggctgtcaggccaagcgcaggcggggctcccagcctcatgataacgtgtaacaggaagggctcccagcctcggtcctcacgtcggtggcaccagaagcgagtgacgacatgtccgtggtcaggaggagcgctggagccagggtgcccagcaggaggtggctgtccccgctgaggtcaccactgcaggggctggcccttgcagtaggggcactcaggtgcggtggccgcacacggctcacagaggatgtggtgctgacagggccgcaggacagcaccgtgggcccgctcccggcaggccacacactgcttggcgcggagctggaagatcacctgcagggccagaacaatgacgggctcaggaggcccctgcccaggctgggccagctggggagccggagggccttccgggcaggactcagcatctcaggccgcccggccaccagctacagagacagggtcagggagacgccccagccagggccacctcagggagaaggcagctgcggggtcagggggtatgctctgtcttaagagccctgggcagaggacagcctcctggggccaccacagacacggcagtggccagacaggccaggcccctgagggcagctgggtgcccaggatgtgggctacgcaggaccctctgtcctatagcagcgagtccaagagcccacccttagcaggcgaggtgaggaggggcacgccgaagtggaggggtcatgtgtgagggtcacagctgtgaacagggagggatggggccacggggagcagtgtggcattcgctgacaagagggaggggtgaaagggcaagggccagcctgacaggaaaggcggctcccagggagacaggcaaggaggccacagggggctgtgctggggtggggcaccgtctcctcctccctaggcccggagcagaccctggggcaggggaggaaggggacaccgcaccagccagggccctccccagacagggacagcagccctgcaggcaggctggcccctcaccctgcccagagcaggactcacgccgtccaccgcctccaggtccaggcgcagctgactctgcagcgagtgcagcttcggcaggggaatggtgccgatgtccccacagccccgcagccccggcagtgtggaggctacgcccaggccctccagctcctcctgcagctgcttcacctgtgcctccacctcctccttcttctgcagcgccagctgccggtcgctatcggccacacgggcacgctccttggcctcctgcgcctctcgctgccaggcatcgcagacctgaaacccagggcccgtctcagcaccccccacctcacctgtgccacctgcagaccctcttgcctgtgtcaccagcgatcctcccctcccctccccagcatcagctgtggccccctcacctgagacccctgcggccctccctccctccctccctccctccctccccctcccgtctcaccccccacacgctcacctgagtcacctgcggccctccctccctcccctcccatctcacccccacacgctcacctgagtcccctgcggccctccctccccctcacctgtctggcccccccacacactcacctgcttcacctgctgccaggactcctcccactgccggatcttcctcttggcctcgtccagctgccgcctgacccgggccagctcagctccgtttggacttgcactcgaagaggatggggggccggcactcaggatgggggaggggctgggggagaagctgccggaaacaaagtcccagatgctcccggggacaccgttcaaacctgagtgtgaaacggtcgatgacggcccagccctgctgtgctcgcggcctggtgggattggtgggtgctatgccccaggccccaccgctacgtgggcaccctctggggctcacctgccctggcagcaggggtgcagccacgcctgccccttgccccactcctggaccccaccaggctcaggaagaggcgctgacagtgacacctgccccggccggtctccccaccagatctacgccttcctggccacctgaggagtctaaacagagaggaattaaattcaagttcctgggaccacaccatgaggtgggaggggcagtggttctcagactccactcccaggaacccctcacacactgagactctcaaagaccccacagagctgtgtccatgtggtgactgccaatgtctatgcagctttagagactatgactgaaatagtttaaatgttacttaaaaaaaacaggccgggcgcggtggctcacgcctgtaatcccagcactttgggaggccgaggcgggtggatcatgaggtcaggagatcgagaccatcctggctaacaaggtgaaaccccgtctctactaaaaatacaaaaaattagccaggcgtggtggcgggcgcctgtagtcccagctactcgggaggctgaggcaggagaatggcgtgaacccgggaagcggagcttgcagtgagccgagattgcgccactgcagtccgcagtccggcctgggcgacagaatgagactccgtctcaaaaaaaaaaaaaaaaaaaaaaaaaaacaggcctgtcatcccagcactttgggaggtcgaggccagcagatcacttgaggtcaggagtttgagaccagtatggccaacatggtgaaactccatctctactaaaaatacaaaaattagcctggcgtggtagcaggtacctgtgatcccagctactcgggaggctgagacatgacaatcattttaacccggaaggcagaggctgcagtgagccaagattgctggagcctggacgacagagtgaaactttgttttaaaaaaataaaaacttttaaaattttgtatttttttgtagactcaggagtctcactatggtgcctagcctggtcttgaacccctggcctcaagtgaccacccacctcgacctcccaaagtgctgggattacaggcttgagccactatgttggcccaaagtattagttttttttttttttttttttttttttttgaaatggagtctagctctatcgccaggctggattggagtgcagtggcacaatctcagctcactgcgacctccgactcccgggttcaagcgattctcctgccttagcctcccaagtagctgggattacaggcatgtgccaccacgcccgcctaatttttgtatttttagtagagacggggtttcaccatgttggccaggatggtctcgatatcctgacgtcgtgatccgcccacctcggcgccccaagtgctgggatgacaggcgtgagccaccacgccgggcctttttctcttttttttttttgagatggagtttcactctt

SEQ ID NO: 29 - DNA, 10 bp, Homo sapiens
gtctacaatt

SEQ ID NO: 30 - protein, 293 aa, Homo sapiens
MTGLYELVWRVLHALLCLHRTLTSWLRVRFGTWNWIWRRCCRAASAAVLAPLGFTLRKPPAVGRNRRHHRHPRGGSCLAAAHHRMRWRADGRSLEKLPVHMGLVITEVEQEPSFSDIASLVVWCMAVGISYISVYDHQGIFKRNNSRLMDEILKQQQELLGLDCSKYSPEFANSNDKDDQVLNCHLAVKVLSPEDGKADIVRAAQDFCQLVAQKQKRPTDLDVDTLASLLSSNGCPDPDLVLKFGPVDSTLGFLPWHIRLTEIVSLPSHLNISYEDFFSALRQYAACEQRLGK

SEQ ID NO: 31 - DNA, 2645 bp, Homo sapiens
ggcacgaggtggcggcgggggcgaggtgaggtgttggcagtggaaaggggttcgggctcggggggcggggggacgcggagcgatggcccgcgccggccgcaggggcggataaaaagccgtcgcgctgcgggagtgggcgggagggagagggggtgtccgagggccacaagagtatgacggggctgtacgagctggtgtggcgggtgctgcacgcgctgctctgtctgcaccgcacgctcacctcctggctccgcgttcggttcggcacctggaactggatctggcggcgctgctgccgcgccgcctctgccgcggtcctagcgccgctcggcttcacgctccgcaagcccccggcagtcggcaggaaccgccgtcaccaccggcacccgcgcggggggtcgtgcctggcagccgcacaccaccggatgcgctggcgcgcggacggtcgttccttggagaagctgcctgtgcatatgggcctggtgatcaccgaggtggagcaggaacccagcttctcggacatcgcgagcctcgtggtgtggtgtatggccgtgggcatctcctacattagcgtctacgaccaccaaggtattttcaaaagaaataattccagattgatggatgaaattttaaaacaacagcaagaacttctgggcctagattgttcaaaatactcaccagaatttgcaaatagtaatgacaaagatgatcaagttttaaattgccatttggcagtgaaggtgctgtctccggaagatggaaaagcagatattgtaagagctgctcaggacttttgccagttagtagcccagaagcaaaagagacccacagatttggatgtagatacgttagccagtttacttagttcaaatggttgtcctgatcctgatttagtattgaagttcggtcctgtggacagcacattaggctttcttccctggcacatcagattgactgagattgtctctttgccttcccacctaaacatcagttatgaggactttttctctgcccttcgtcaatatgcagcctgtgaacagcgtctgggaaagtagtggtcattggttgcataatttgatttgaggcttgtggaggaaaggaaccaagtgactctgatgtttacaaagcacctatgaaaccctgtacacacctagttcataatcctcataatttatcaacaaacacaaaaaagtgtcttacttgagagtgagtgtgtgtgtgtgcgtgtgcacgtgcacacatgtgcacgtttgtatgtatggaaataaacttataaatggggacgtattggagaaggaaatacatagacctacaactttgagcaaatagcagtgatgttttaggaactgaaatgtcacacttaaagtcttcagcccagctacttccctatttttgtggggagaagagggcctgattagaactgttctggttgtgtttggcgggaggggaataatttttgttcagtccttcttagtgaccaaactttaatttttaagaataatatattgacttactgaactgaagcattctgagttgaaaggagctccagaggagtggagttctgtgttgctcacatgttaaaatcttgctcaccttcagagcagagggaatacctatcttcagatatccgtccattttcatctcttaattgtagtcaaaagtatgacttgagagtgttgctctggtattctgggttctgaagtctggtattctggtattctgggttcaaaagtatgacttgagagtgttgctctggtattctgagagttgctctgtattctgggttctgaagattatttgaaaaataactcctactacattgaaatgcagacttaaaaatttaaacattggattaggcagtcaaaaaaaccaagcaagcataaaaggtcaataagttgtaatcttgatagtaaaggtggaaaacttattataaatggaaagaaagttttatttccttttttgtttgatgggcagtatgccatattatacccaaagttcttttaaaaaatatttccatcaaccatttttatttaaaataaacatttgagggaagttaccaaggcagcttttttcctcaaaagtaacctgttcctctttggaatagcacattttaggggcatggttaatacctgagatttttactcagtaaatcctgatggttactgtgtgtaaaatatctttaagtaggattgaaggcctctgtgggggaataaaatattaccaaagtctataaaaataaattttacatgttctcttttatgacagagagcagcactggttctgttatttttaaaatgaataattgatttcttgataggtgtttaatatttcttccctcactgctgattcttagatagaaaccattctttatatttgatagactgctttcagaaaacccttatcaacaagtgtacaatacttatctaaaactatacatttagaatggagcagtttaatactagatctcagaagttttgaaaaatagcaaagaagactggatttggaaagcatggtctacaattggttgttaaattctgaagctatgaagaataaatgtttcaactttggattatgaaaccccatttatgattttttaaatacacttgaaataaaaatgattaaactaaaaaaaaaaaaaaaaaaaa

SEQ ID NO: 32 - DNA, 10 bp, Homo sapiens
gggctcgggg

SEQ ID NO: 33 - protein, 323 aa, Homo sapiens
MQAAWLLGALVVPQLLGFGHGARGAEREWEGGWGGAQEEEREREALMLKHLQEALGLPAGRGDENPAGTVEGKEDWEMEEDQGEEEEEEATPTPSSGPSPSPTPEDIVTYILGRLAGLDAGLHQLHVRLHALDTRVVELTQGLRQLRNAAGDTRDAVQALQEAQGRAEREHGRLEGCLKGLRLGHKCFLLSRDFEAQAAAQARCTARGGSLAQPADRQQMEALTRYLRAALAPYNWPVWLGVHDRRAEGLYLFENGQRVSFFAWHRSPRPELGAQPSASPHPLSPDQPNGGTLENCVAQASDDGSWWDHDCQRRLYYVCEFPF

SEQ ID NO: 34 - DNA, 1407 bp, Homo sapiens
cgaccaacggaccggacagagacgaggagaggaacaggaagagagaagctgggagaatcgggaacctgggggctagtgacctgcacacagggcaggggcactcggcagttcccagaggccacccctcccaccccagacatccagacatctggaactttgggtgccaagagtccagcttaatgcaggcagcctggcttttgggggctttggtggtcccccagctcttgggctttggccatggggctcggggagcagagagggagtgggagggaggctggggaggtgcccaggaggaggagcgggagagggaggccctgatgctgaagcatctgcaggaagccctaggactgcctgctgggaggggggatgagaatcctgccggaactgttgagggaaaagaggactgggagatggaggaggaccagggggaggaagaggaggaggaagcaacgccaaccccatcctccggccccagcccctctcccacccctgaggacatcgtcacttacatcctgggccgcctggccggcctggacgcaggcctgcaccagctgcacgtccgtctgcacgcgttggacacccgcgtggtcgagctgacccaggggctgcggcagctgcggaacgcggcaggcgacacccgcgatgccgtgcaagccctgcaggaggcgcagggtcgcgccgagcgcgagcacggccgcttggagggctgcctgaaggggctgcgcctgggccacaagtgcttcctgctctcgcgcgacttcgaagctcaggcggcggcgcaggcgcggtgcacggcgcggggcgggagcctggcgcagccggcagaccgccagcagatggaggcgctcactcggtacctgcgcgcggcgctcgctccctacaactggcccgtgtggctgggcgtgcacgatcggcgcgccgagggcctctacctcttcgaaaacggccagcgcgtgtccttcttcgcctggcatcgctcaccccgccccgagctcggcgcccagcccagcgcctcgccgcatccgctcagcccggaccagcccaacggtggcacgctcgagaactgcgtggcgcaggcctctgacgacggctcctggtgggaccacgactgccagcggcgtctctactacgtctgcgagttccccttctagcggggccggtaccccgcctccttgcccatcccaccacccggcctttccctgcgccgtgcccaccctcctccggaatcgcccttcccttcctggccacgaatggcagcgtcctccccgacccccagtctgggcgcttctgggagggctcttgcggtgccggcactcctccttgttagtgtctttccttgaaggggcgggcaccaggctaggtccggtgccaataaatccttgtggaatctgaaaaaaaaaaaaaaaa

SEQ ID NO: 35 - DNA, 10 bp, Homo sapiens
tgcaggtggc

SEQ ID NO: 36 - protein, 204 aa, Homo sapiens
MGLGAVPGRQAAFREGLEQAVRYAKALGCPRIHLMAGRVPQGADRIAVKAEMEAVFLENLRHAAGVLAQEDLVGLLEPINTRITDPQYFLDTPQQAAAILQKVGRPNLQLQMDIFHWQIMDGNLTGNIREFLPIVGHVQVAQVPGRGEPSSPGELNFPYLFQLLEDEGYKGFVGCEYQPRGDTVEGLSWLRSYWDRRGHPEAGQ

SEQ ID NO: 37 - DNA, 999 bp, Homo sapiens
tttttttggcggtataaaagtgcttctttatttggtcatacagctttcaaatgttgcttaacgaacagatagcatttttataatcagaggaaagactcagtattaaagtgcgcgtcccatcattgcaaccgggccctcgggcagccagctctctcttgcttcgcgtccggctctcaggccctggagctcggcctccctaccttctccaggcgcgggtcctgctccttttccggccgcccttcctcagcccccggctcggacactctgcaggagaccaagagaagggggaaatggggctgggggccgtccccgggagacaggcggccttccgagagggactggagcaggccgtgcggtatgccaaagccctgggctgtcccaggatccacctgatggctggccgagtaccccagggagctgatcgaatagcagtcaaggctgagatggaggccgtttttctggagaacctgaggcatgcagctggggttttggctcaggaggacctcgtgggactgctggagcccatcaacacccgcatcactgacccccagtacttcctggacacgccccagcaggcggcagccatcttacagaaggtaggaagacccaacctccaattacaaatggacatattccactggcagatcatggatgggaacctgacaggaaacatccgggagttcctgcccattgttgggcatgtgcaggtggcacaggtcccaggccgaggggagcccagcagccccggagagctgaatttcccctatctgtttcaactgctggaagatgaaggctacaaaggcttcgtgggctgtgagtatcagcctcgaggagacacagtagagggcttgagttggctacgttcatactgggataggcggggccacccagaggctggccagtgagggcccgcacaccacccacgtgcctccagacagcgagtgacatcccatctcctcctctgcattaaagatgacctgctgaaaaaaaaaaaaaaaa

SEQ ID NO: 38 - DNA, 10 bp, Homo sapiens
ctgctaaccc

SEQ ID NO: 39 - protein, 511 aa, Homo sapiens
MLVDGPSERPALCFLLLAVAMSFFGSALSIDETRAHLLLKEKMMRLGGRLVLNTKEELANERLMTLKIAEMKEAMRTLIFPPSMHFFQAKHLIERSQVFNILRMMPKGAALHLHDIGIVTMDWLVRNVTYRPHCHICFTPRGIMQFRFAHPTPRPSEKCSKWILLEDYRKRVQNVTEFDDSLLRNFTLVTQHPEVIYTNQNVVWSKFETIFFTISGLIHYAPVFRDYVFRSMQEFYEDNVLYMEIRARLLPVYELSGEHHDEEWSVKTYQEVAQKFVETHPEFIGIKIIYSDHRSKDVAVIAESIRMAMGLRIKFPTVVAGFDLVGHEDTGHSLHDYKEALMIPAKDGVKLPYFFHAGETDWQGTSIDRNILDALMLNTTRIGHGFALSKHPAVRTYSWKKDIPIEVCPISNQVLKLVSDLRNHPVATLMATGHPMVISSDDPAMFGAKGLSYDFYEVFMGIGGMKADLRTLKQLAMNSIKYSTLLESEKNTFMEIWKKRWDKFIADVATK

SEQ ID NO: 40 - protein, 270 aa, Homo sapiens
MDSLEWNWALVYELSGEHHDEEWSVKTYQEVAQKFVETHPEFIGIKIIYSDHRSKDVAVIAESIRMAMGLRIKFPTVVAGFDLVGHEDTGHSLHDYKEALMIPAKDGVKLPYFFHAGETDWQGTSIDRNILDALMLNTTRIGHGFALSKHPAVRTYSWKKDIPIEVCPISNQVLKLVSDLRNHPVATLMATGHPMVISSDDPAMFGAKGLSYDFYEVFMGIGGMKADLRTLKQLAMNSIKYSTLLESEKNTFMEIWKKRWDKFIADVATK

SEQ ID NO: 41 - DNA, 3071 bp, Homo sapiens
gtcctaatagccacgcccacctcaaatggcgatcccagagagcatctggcttcaattttcctttttccggaggggagatggattccctggaatggaactgggctctggtgtatgagctcagtggagagcaccatgacgaagagtggtcagtgaagacttaccaggaagtagctcagaagtttgtggaaactcaccctgagtttattggaatcaaaatcatttattcggatcacagatccaaagatgtggctgtcatcgcagaatccatccgaatggccatggggctccgaatcaagttccccacggtggtggcagggtttgacctggtggggcatgaggacactggccactccttgcgtgactacaaggaagctctgatgatccccgccaaggatggcgttaagctgccttacttcttccacgccggaggaacagactggcagggtacttccatagacaggaacattctggatgctctgatgctgaacactaccagaatcggccatggatttgctttgagcaaacaccccgcagtcaggacttactcctggaaaaaggacatccccatagaagtctgtcccatctctaaccaggtgctgaaactggtgtctgacttgaggaaccaccctgtagccactctgatggccactgggcaccccatggtgatcagctctgatgacccagctatgtttggtgccaaaggcttgtcctatgatttctatgaggtcttcatgggcattggggggatgaaggctgacctgaggaccctcaaacagctggccatgaactctatcaagtacagtaccctgttggagagtgagaaaaatactttcatggaaatctggaagaagagatgggataagttcatagcagatgtggctacaaagtgaggagaagctagccagccctctacaagctgtcctcttgcacacgctgtcacttcctctcactcgttcttgaatcagctccatgtgcccatgaaatcaatggcctctgtatggagcgaccctgtgagaagcacttggctggctgagcaaattcatcctctggaaatattctctctcagccacagtgacattgaccctcttggttttctcctgtctctggccatttcttccagtttccctatttcagagtcttctcctctctctgatctctgtgctgtttcctcaggactcagtcctgggctctcttctattctggtctctttatttttttatttttgtattttttcgagatggagttttgctcttgttgcccaggctggagtacaatggtgcgatctcagctcagtgcaacctccgccacccgggttcaggcaattctcttgcatcagcctcgcgagtagttggaattataggcatgtgccaccacacccagctgatttttgcatttttagtagagacaggttttcaccatgttggcgaggctggtatccaactcttgacctcaggtgatccactcgccccttggctcccaaagtgctggaattacaggcattagccaccatgcctggcctattctggtctctttaactctctcctctttatttctcttctctctctgtacacttttcctgggtggtctcatccattcctttgctttttcataccatttatttgttaatgattcccacatttatttatgcacttggagagctcacaggaatctcagaaactgatgaggtacaattctgaaccctcagtctcttccctttaaacctttctttttctctactttaatttttctaaagagtgtcttgctatgttgcccaggctggtctccaactcaagtgatcctcctgccgcagtctcccgaagtgctgggattactgacatgagccaccacactcagccctttaaacctttccctggcctttcccatagctggtgaaggacacctccatccattccacgcagttgctcaaagcagaaattttcagtgcaagtcttgatgctgcgccgtcccccactccctacatcagaacgcatccctcatctggactccagcggtggcttcttgatgctgcgcggtcccccactccctacatcagaatgcatcccgcatccagactccagcggtggtgctctacctgcacgctgttgccaagtccaagctaccatactcctgcctgagctatgacaacagcctcctcactgatctcccctttcttccctttgcctcctccagctcatttttcacagtgtagaatgacattttgtttgtttgttttgttttgtttgagatggagtctcgctctgttgcccagggtggagtgcagcggtgcgatctcggctcactgcaacctccacctcccgggttcaagcggattctcgtgcctcagcctcctgagtagctgggattacaggcatgcaccaccatgcccggataatttttgtatttttagtagagatggggtttcactatgttggccaggctggtctcgaacacctgacctcgtggtccacccgcctcggcctcccaaagcactgggattacaggcgtgagccacccggcctggcctagaatgacttttaaaagatcaaattaaatcaggtcactcctttgcttacaacgcagtgcgtttagaggtacaccccatgtctccacagggcatacagcatccgatttaatctggatccattccggcgccttcctctcccagtcacccagagggccccaaccccggcggccctttcttcctcaaatgtcctcggctctataccgtgcctgggtcttttctctttctctctgcctggaagattccttctttccccttttgtcttgcccactcctgtttacccttcaagtttcaagttcatgtcactgtctcagagaggttttcctgtgctcgccctgtttctctcaggaagccttgctcttttccatcatgcctctaatcacagcttataatcggatatttatttctgtgtctacagtcttgccctgccagactgtatgccccatgtgggcaggcgctcatgattgtttctgattgtttcacgcatgctgctaacccagagcctgggcccaaagctagttagtactcaataaacaatgcattgaatgag

SEQ ID NO: 42 - DNA, 10 bp, Homo sapiens
agcctgcaga

SEQ ID NO: 43 - protein, 173 aa, Homo sapiens
MAAPSGGWNGVGASLWAALLLGAVALRPAEAVSEPTTVAFDVRPGGVVHSFSHNVGPGDKYTCMFTYASQGGTNEQWQMSLGTSEDHQHFTCTIWRPQGKSYLYFTQFKAEVRGAEIEYAMAYSKAAFERESDVPLKTEEFEVTKTAVAHRPGAFKAELSKLVIVAKASRTEL

SEQ ID NO: 44 - DNA, 10 bp, Homo sapiens
aaaagaaatg

SEQ ID NO: 45 - protein, 142 aa, Homo sapiens
MLDAACPQTSDCKFFSFWTLGLTPVICQELSGLQQQTEGCAVGFPTFEVLGLRLASWLFSLQMAYCGTSPCDHLARASWGNGAPGVVVQTSASLQPVCGGARCSRSLRTAGRPAHPWKRELAAWSTPPRRARTAGTPMFLPL

SEQ ID NO: 46 - DNA, 1972 bp, Homo sapiens
aatagcaggagtggcctgtgcctcaacaacatgcttttgctgcagataatcagccagagcctgtttctctactcctcgctaagaatgctttgtttcccgtaaggaaagcttttagctaatctataatctatagaaactatgcttatcattggctgtcagtaaatatgtgggtcagactctgtttgtggctttcagctctgaaggctgtcagctccctgatcccactctgcactctatttctgtgtctgtctttaattcttctagtgctgctgggttagggtcttcacgaccgaactggtctcggcagtggaagaacatggaaacactaggctgacttagtctcccagcctacatctttctcccatgctggacgctgcctgccctcaaacatcagactgcaagttcttcagcttttggactcttggacttacaccagtgatttgccaggagctctcaggccttcagcaacagactgaaggctgtgctgtcggcttccctacttttgaggttttgggactcagactggcttcctggctcttcagcttgcagatggcctattgtgggacttcaccttgtgatcatctcgcccgtgcttcgtgggggaatggagcccctggagtggttgtgcagaccagtgcaagcctacaacccgtgtgcggaggcgctcggtgcagcaggagcctcagaacggcggggcgccctgcccacccctggaagagagagctggctgcctggagtactccaccccgcagggccaggactgcgggcacacctatgttcctgcctttataactacctctgcattcaacaaggagagaacacgacaagctacgtctccacactggtctacacacacagaggatgctggatactgtatggagtttaagacagagtccttgactcctcactgtgctctggaaaaccggcccttgactagatggatgcagtatctccgagagggatacacggtgtgtgtggattgtcagcctccagctatgaactctgtgagccttcgttgttctggagatggcctggactccgatggaaatcagactctccattggcaagcaattggtaatcctcggtgtcaaggaacttggaaaaaagttcggcgagtagaccagtgttcttgtccagctgttcacagttttatttttatatagatggtgatataaatatttccaaatgcatttgtaaacattctaaatattctcaagtcatgttcaatgtttcctaaaccttcaattttggccaaagtccccaaacacatcattgccacactctgaagtagagaaagaaaatttaggggccagttctcaaggaacacaggtcctttatttttattttaactaagttgaagacccactcaaaaagctcttgtggttttatgttcttgacctttcaactggagtcctctcattcagcaggtggcccgtgagacacagaatacatgtctgtttgctaaagtaaaattactgtaactcagtccgattattggtgacggaagtgtcatttaaggggatctatgttttgaaccttgcagtctattttataatcttttaaaagtttctcactttaaaacatgtatacaggtatgcacactctacacatatatttccatatacttaatattccataaattctgaaataatttcaagcaaattatcacaaataatttttccacagggcaaattatttaaaattttagtaagcattctgtaatgaaaaaccaagttatactaaaaacattttttgaagaagaaattttttttttgcttaagaagtgaaaggatcaaatgctcattgtaggtcaatgggagttcttttaaatgttataattttcatgaaaagaaatgttgacaccagtgaatgaaacaattgctttcattctgaaaattctaccaccatttgcatctaagattatttccaaggcttaaagcctgaagctgaataaaataatctttcagagtcc

SEQ ID NO: 47 - DNA, 10 bp, Homo sapiens
atgacctgaa

SEQ ID NO: 48 - protein, 332 aa, Homo sapiens
MLLLAAAFLVAFVLLLYMVSPLISPKPLALPGAHVVVTGGSSGIGKCIAIECYKQGAFITLVARNEDKLLQAKKEIEMHSINDKQVVLCISVDVSQDYNQVENVIKQAQEKLGPVDMLVNCAGMAVSGKFEDLEVSTFERLMSINYLGSVYPSRAVITTMKERRVGRIVFVSSQAGQLGLFGFTAYSASKFAIRGLAEALQMEVKPYNVYITVAYPPDTDTPGFAEENRTKPLETRLISETTSVCKPEQVAKQIVKDAIQGNFNSSLGSDGYMLSALTCGMAPVTSITEGLQQVVTMGLFRTIALFYLGSFDSIVRRCMMQREKSENADKTA

SEQ ID NO: 49 - DNA, 2272 bp, Homo sapiens
aggcgcccgcccgccgcgcgtgattctcgcctcgccgcagcccagccctgcgcgccttgcccggcggcccccgcccggccgctccgggcccctggccccgcggagcgatgctgctgctggctgccgccttcctcgtggccttcgtgctgctgctgtacatggtgtctccgctcatcagccccaagcccctcgccctgcccggggcgcatgtggtggttacaggaggttccagtggcatcgggaagtgcattgctatcgagtgctataaacaaggagcttttataactctggttgcacgaaatgaggataagctgctgcaggcaaagaaagaaattgaaatgcactctattaatgacaaacaggtggtgctttgcatatcagttgatgtatctcaagactataaccaagtagagaatgtcataaaacaagcacaggagaaactgggtccagtggacatgctggtaaattgtgcaggaatggcagtgtcaggaaaatttgaagatcttgaagttagtacctttgaaaggttaatgagcatcaattacctgggcagcgtgtaccccagccgggccgtgatcaccaccatgaaggagcgccgggtgggcaggatcgtgtttgtgtcctcccaggcaggacagttgggattattcggtttcacagcctactctgcatccaagtttgccataaggggattggcagaagctttgcagatggaggtgaagccatataatgtctacatcacagttgcttacccaccagacacagacacacctggctttgccgaagaaaacagaacaaagcctttggagactcgacttatttcagagaccacatctgtgtgcaaaccagaacaggtggccaaacaaattgttaaagatgccatacaaggaaatttcaacagttcccttggctcagatgggtacatgctctcggccctgacctgtgggatggctccagtaacttctattactgaggggctccagcaggtggtcaccatgggccttttccgcactattgctttgttttaccttggaagttttgacagcatagttcgtcgctgcatgatgcagagagaaaaatctgaaaatgcagacaaaactgcctaatcttcttaccccttggaagaagactgtttccaaataatttgaacagcttgctgctaaatgggacccaatttttggcctatagacacttatgtattgttttcgaatacgtcagattggaccagtgctcttcaggaatgtggctgcaagcaaggggctagaagttcacctcctgacagtattattaatactatgcaaatatggaataggagaccatttgattttctaggctttgtggtagagaggtgaaggtatgagaattaatagcgtgtgaacaaagtaaagaacaggattccagaatgatcattaaatttgtttctatttattcttttttgcccccctagagattaagtccagaaatgtactttctggcacataaagaaatcttgaggactttgtttaaaccttccataaaaaaacaattttcggtttctcgggttctctctctctgtctctctgtctctctgtctctctgtctctctgtctctctctctctctctctttctttctttgtgtattttattcaagatgagttggacccattgccagtgagtctgaatgtcactgacagccctgtgttgtgctcaggactcactctgctgctggtggaaactcatggcttctctctctctttgatcccataaagctacgagggggacgggagagggcagtgcaatgggaagtaaagagatattttccagtaggaaaagcaatgctttcttgtctttagactcaaatgcttagggaacgtttcatttctcattcatggggaaaggcagcctccttaaatgttttctgaagagcggtaaaatctagaagcttaagaatttacagttccttcaataaccatgatgacctgaagttcacctatcccattttagcatctacttgtttttcccatctcttcctttccaattttgcttatactgctgtaatatttttgtaaaaaaaaaaaaaaaggaaaaaaaagaccagctaaaattttcgacttgactttttaacttaactcatgaattaattaaagcaaatgaaaaaattaaaaagtgtgactttttctcggagcatatatgtagcttttaggaaaggctgatgatggtataaagtttgctcattaagaaaaaaagacaaggctgattttgaagagagttgcttttgaaataaaatgatca

SEQ ID NO: 50 - DNA, 10 bp, Homo sapiens
tttaatacat

SEQ ID NO: 51 - protein, 160 aa, Homo sapiens
MQRVSGLLSWTLSRVLWLSGLSEPGAARQPRIMEEKALEVYDLIRTIRDPEKPNTLEELEVVSESCVEVQEINEEEYLVIIRFTPTVPHCSLATLIGLCLRVKLQRCLPFKHKLEIYISEGTHSTEEDINKQINDKERVAAAMENPNLREIVEQCVLEPD

SEQ ID NO: 52 - DNA, 1019 bp, Homo sapiens
ggcacgagggtctggccccgccttttctctgctctcctgaacctttaggcttgtctcggcccatttgaagaccaggaagttgatcaatcccgaggctgctgagagacggtggcgcgattgggacagtcgccagggatggctgagcgtgaagatgcagcgggtgtccgggctgctctcctggacgctgagcagagtcctgtggctctccggcctctctgagccgggagctgcccggcagccccggatcatggaagagaaagcgctagaggtttatgatttgattagaactatccgggacccagaaaagcccaatactttagaagaactggaagtggtctcggaaagttgtgtggaagttcaggagataaatgaagaagaatatctggttattatcaggttcacgccaacagtacctcattgctctttggcgactcttattgggctgtgcttaagagtaaaacttcagcgatgtttaccatttaaacataagttggaaatctacatttctgaaggaacccactcaacagaagaagacatcaataagcagataaatgacaaagagcgagtggcagctgcaatggaaaaccccaacttacgggaaattgtggaacagtgtgtccttgaacctgactgatagctgttttaagagccactggcctgtaattgtttgatatatttgtttaaactctttgtataatgtcagagactcatgtttaatacataggtgatttgtacctcagagcattttttaaaggattctttccaagcgagatttaattataaggtagtacctaatttgttcaatgtataacattctcaggatttgtaacacttaaatgatcagacagaataatattttctagttattatgtgtaagatgagttgctatttttctgatgctcattctgatacaactatttttcgtgtcaaatatctactgtgcccaaatgtactcaatttaaatcattactctgtaaaataaataagcagatgattcttataaaaaaaaaaaaaaaaaa

SEQ ID NO: 53 - DNA, 10 bp, Homo sapiens
tgtaaacaat

SEQ ID NO: 54 - protein, 375 aa, Homo sapiens
MKVWLLLGLLLVHEALEDVTGQHLPKNKRPKEPGENRIKPTNKKVKPKIPKMKDRDSANSAPKTQSIMMQVLDKGRFQKPAATLSLLAGQTVELRCKGSRIGWSYPAYLDTFKDSRLSVKQNERYGQLTLVNSTSADTGEFSCWVQLCSGYICRKDEAKTGSTYIFFTEKGELFVPSPSYFDVVYLNPDRQAVVPCRVTVLSAKVTLHREFPAKEIPANGTDIVYDMKRGFVYLQPHSEHQGVVYCRAEAGGRSQISVKYQLLYVAVPSGPPSTTILASSNKVKSGDDISVLCTVLGEPDVEVEFTWIFPGQKDERPVTIQDTWRLIHRGLGHTTRISQSVITVEDFETIDAGYYICTAQNLQGQTTVATTVEFS

SEQ ID NO: 55 - DNA, 1502 bp, Homo sapiens
cctgcgtccccgccccgcgcagccgccgcgctcctgcgctccgaggtccgaggttcccgagatgaaggtctggctgctgcttggtcttctgctggtgcacgaagcgctggaggatgttactggccaacaccttcccaagaacaagcgtccaaaagaaccaggagagaatagaatcaaacctaccaacaagaaggtgaagcccaaaattcctaaaatgaaggacagggactcagccaattcagcaccaaagacgcagtctatcatgatgcaagtgctggataaaggtcgcttccagaaacccgccgctaccctgagtctgctggcggggcaaactgtagagcttcgatgtaaagggagtagaattgggtggagctaccctgcgtatctggacacctttaaggattctcgcctcagcgtcaagcagaatgagcgctacggccagttgactctggtcaactccacctcggcagacacaggtgaattcagctgctgggtgcagctctgcagcggctacatctgcaggaaggacgaggccaaaacgggctccacctacatcttttttacagagaaaggagaactctttgtaccttctcccagctacttcgatgttgtctacttgaacccggacagacaggctgtggttccttgtcgggtgaccgtgctgtcggccaaagtcacgctccacagggaattcccagccaaggagatcccagccaatggaacggacattgtttatgacatgaagcggggctttgtgtatctgcaacctcattccgagcaccagggtgtggtttactgcagggcggaggccgggggcagatctcagatctccgtcaagtaccagctgctctacgtggcggttcccagtggccctccctcaacaaccatcttggcttcttcaaacaaagtgaaaagtggggacgacatcagtgtgctctgcactgtcctgggggagcccgatgtggaggtggagttcacctggatcttcccagggcagaaggatgaaaggcctgtgacgatccaagacacttggaggttgatccacagaggactgggacacaccacgagaatctcccagagtgtcattacagtggaagacttcgagacgattgatgcaggatattacatttgcactgctcagaatcttcaaggacagaccacagtagctaccactgttgagttttcctgacttggaaaaggaaatgtaatgaacttatggaaagcccatttgtgtacacagtcagctttggggttccttttattagtgctttgccagaggctgatgtcaagcaccacaccccaaccccagcgtctcgtgagtccgacccagacatccaaactaaaaggaagtcatccagtctattcacagaagtgttaacttttctaacagaaagcatgattttgattgcttacctacatacgtgttcctagtttttatacatgtgtaaacaattttatataatcaatcatttctattaaatgagcacgtttttgtaaaaaat

SEQ ID NO: 56 - DNA, 10 bp, Homo sapiens
ggaccgaggg

SEQ ID NO: 57 - protein, 194 aa, Homo sapiens; identical to SEQ ID NO: 19

SEQ ID NO: 58 - DNA, 1841 bp, Homo sapiens; identical to SEQ ID NO: 20

SEQ ID NO: 59 - DNA, 10 bp, Homo sapiens
tccaacccag

SEQ ID NO: 60 - protein, 355 aa, Homo sapiens
MGNLFMLWAALGICCAAFSASAWSVNNFLITGPKAYLTYTTSVALGAQSGIEECKFQFAWERWNCPENALQLSTHNRLRSATRETSFIHAISSAGVMYIITKNCSMGDFENCGCDGSNNGKTGGHGWIWGGCSDNVEFGERISKLFVDSLEKGKDARALMNLHNNRAGRLAVRATMKRTCKCHGISGSCSIQTCWLQLAEFREMGDYLKAKYDQALKIEMDKRQLRAGNSAEGHWVPAEAFLPSAEAELIFLEESPDYCTCNSSLGIYGTEGRECLQNSHNTSRWERRSCGRLCTECGLQVEERKTEVISSCNCKFQWCCTVKCDQCRHVVSKYYCARSPGSAQSLGRVWFGVYI

SEQ ID NO: 61 - protein, 1597 aa, Homo sapiens
CAGAATTTTCTCACATAAATACTGAGGAAGACCCTGCCCTCTCCTCACTCCTCTGGACTTGGCCCTGAGCTGGACCTGGTCCACTGGGGTAGGCAGGGCGATGGGGAACCTGTTTATGCTCTGGGCAGCTCTGGGCATATGCTGTGCTGCATTCAGTGCCTCTGCCTGGTCAGTGAACAATTTCCTGATAACAGGTCCCAAGGCCTATCTGACCTACACGACTAGTGTGGCCTTGGGTGCCCAGAGTGGCATCGAGGAGTGCAAGTTCCAGTTTGCTTGGGAACGCTGGAACTGCCCTGAAAATGCTCTTCAGCTCTCCACCCACAACAGGCTGAGAAGTGCTACCAGAGAGACTTCCTTCATACATGCTATCAGCTCTGCTGGAGTCATGTACATCATCACCAAGAACTGTAGCATGGGTGACTTCGAAAACTGTGGCTGTGATGGGTCAAACAATGGAAAAACAGGAGGCCATGGCTGGATCTGGGGAGGCTGCAGCGACAATGTGGAATTTGGGGAAAGGATCTCCAAACTCTTTGTGGACAGTTTGGAGAAGGGGAAGGATGCCAGAGCCCTGATGAATCTTCACAACAACAGGGCCGGCAGACTGGCAGTGAGAGCCACCATGAAAAGGACATGCAAATGTCATGGCATCTCTGGGAGCTGCAGCATACAGACATGCTGGCTGCAGCTGGCTGAATTCCGGGAGATGGGAGACTACCTAAAGGCCAAGTATGACCAGGCGCTGAAAATTGAAATGGATAAGCGGCAGCTGAGAGCTGGGAACAGCGCCGAGGGCCACTGGGTGCCCGCTGAGGCCTTCCTTCCTAGCGCAGAGGCGGAACTGATCTTTTTAGAGGAATCACCAGATTACTGTACCTGCAATTCCAGCCTGGGCATCTATGGCACAGAGGGTCGTGAGTGCCTACAGAACAGCCACAACACATCCAGGTGGGAGCGACGTAGCTGTGGGCGCCTGTGCACTGAGTGTGGGCTGCAGGTGGAAGAGAGGAAAACTGAGGTCATAAGCAGCTGTAACTGCAAATTCCAGTGGTGCTGTACGGTCAAGTGTGACCAGTGTAGGCATGTGGTGAGCAAGTATTACTGCGCACGCTCCCCAGGCAGTGCCCAGTCCCTGGGGAGAGTTTGGTTTGGGGTCTATATCTAGAGGGACCTTCAAAGTATTTGTTCCTTTAAATTTCAGACCATGTCCAACCCAGCTGTGCTGCTGGGAATCAGGAGAATAGAAGCAAAAAACGAAAGAGTTCTGTTCAGACTTCTGAAGAGCAGCCTGTGGCTACAAATCTATGCTGATAAATGAGATTGAGAACTCAACTGTATTTTGCCATAAATGCTTCTAAGATATATCCAGCTGGGACTTCTATTACTCCCTTTGGAAACCTTAAGATCAAAAAGGGAATAAGAAACCCTTCTTCTGTATCCCAATAATCCACCAGGATAAAGGAGAAACTAGAAATATGCAACTCCCTTGATTTCAGTGTTTGGCAGGTAACAAAAAATTGAGACCCAGACACTGGTCAACAGGAAAACAATACAGACTCCCAGAATTAGAAAGTGTTATTTTAATGCAACCTAG

SEQ ID NO: 62 - DNA, 10 bp, Homo sapiens
actcgaatat

SEQ ID NO: 63 - protein, 248 aa, Homo sapiens
MQTCPLAFPGHVSQALGTLLFLAASLSAQNEGWDSPICTEGVVSVSWGENTVMSCNISNAFSHVNIKLRAHGQESAIFNEVAPGYFSRDGWQLQVQGGVAQLVIKGARDSHAGLYMWHLVGHQRNNRQVTLEVSGAEPQSAPDTGFWPVPAVVTAVFILLVALVMFAWYRCRCSQQRREKKFFLLEPQMKVAALRAGAQQGLSRASAELWTPDSEPTPRPLALVFKPSPLGALELLSPQPLFPYAADP

SEQ ID NO: 64 - DNA, 2000 bp, Homo sapiens
attttcctggggctccggggcgcggagaagctgcatcccagaggagcgcgtccaggagcggacccgggagtgtttcaagagccagtgacaaggaccaggggcccaagtcccaccagccatgcagacctgccccctggcattccctggccacgtttcccaggcccttgggaccctcctgtttttggctgcctccttgagtgctcagaatgaaggctgggacagccccatctgcacagagggggtagtctctgtgtcttggggcgagaacaccgtcatgtcctgcaacatctccaacgccttctcccatgtcaacatcaagctgcgtgcccacgggcaggagagcgccatcttcaatgaggtggctccaggctacttctcccgggacggctggcagctccaggttcagggaggcgtggcacagctggtgatcaaaggcgcccgggactcccatgctgggctgtacatgtggcacctcgtgggacaccagagaaataacagacaagtcacgctggaggtttcaggtgcagaaccccagtccgcccctgacactgggttctggcctgtgccagcggtggtcactgctgtcttcatcctcttggtcgctctggtcatgttcgcctggtacaggtgccgctgttcccagcaacgccgggagaagaagttcttcctcctagaaccccagatgaaggtcgcagccctcagagcgggagcccagcagggcctgagcagagcctccgctgaactgtggaccccagactccgagcccaccccaaggccgctggcactggtgttcaaaccctcaccacttggagccctggagctgctgtccccccaacccttgtttccatatgccgcagacccatagccgcctgcaaggcagagaggacacaggagagccagccctgagtgccgaccttgggtggcggggcctgggtctctcgtcccacccggagggcacagacaccggcttgcttggcaggctgggcctctgtgtcacccactcctgggtgcgtgcagacccttcccctccaccccccaggtcttccaagctctgcttcctcagtttccaaaatggaaccacctcacctccgcagcacccgacttaccaggacgcatgcccctccctctgccctcatcaaacccacagacccggactccctttctgccaccccaggctggtccggccccaggtgtggggtccgctctctccactcccagggctccgcgcccaagtgagggggcccctgccggagcctcagacacactggagttcagggctgggggggccttggcacatacctgtcccttggctatgagcaggctttgggggcccttccgcggcagccccgggggccgaggtagggtctgggggcttagaggctgggatggctcctggccccaccgccagggggcaagcgcaggccgggctgggaggcggcggcggcggctcgggctggggggtcaggtggacgctgcctccggggctggtcgcgcatccctcagtccctcggccacccgggggtcgctccctcgtgcccaccgcacctgccgagcctctttggacccagatctgttcatgcttttgtcttcgtcactgcggcggggccctttgatgtcttcatctgtatggggtggaaaaatcaccgggaatcccccttcagttctttgaaaaagttccatgactcgaatatctgaaatgaagaaaacaaaccgactcacaaacctccaagtagctccaaatgcaatttttaaaatggaaaacaaaaatctgaaagaaacgtctttagtggctttaagccccaaaacgtccctaaggcgtcctcgagatgaagacgggggggagcccccagccaggtggagaccccgcaggacgcggcggcgcccggtgaccgaggcctcgcacagccggccgccctgagggtcgggccggagccagggtccaagaggggcgcgtttgtgtctcgggttaaaataaggttccgtccgcgtgctgggtcaga